SEQUENCE LISTING (12 sequences)

SEQ ID NO: 1 - DNA, 20 bp, synthetic construct
  source 1..20: mol_type = other DNA
catggccttccgtgttccta

SEQ ID NO: 2 - DNA, 17 bp, synthetic construct
  source 1..17: mol_type = other DNA
gcggcacgtcagatcca

SEQ ID NO: 3 - DNA, 21 bp, synthetic construct
  source 1..21: mol_type = other DNA
tcctcttgggcatcatcttcc

SEQ ID NO: 4 - DNA, 21 bp, synthetic construct
  source 1..21: mol_type = other DNA
tttgtagtggatcgtgcctcg

SEQ ID NO: 5 - DNA, 21 bp, synthetic construct
  source 1..21: mol_type = other DNA
ccaagtgctgccgtcattttc

SEQ ID NO: 6 - DNA, 21 bp, synthetic construct
  source 1..21: mol_type = other DNA
ggctcgcagggatgatttcaa

SEQ ID NO: 7 - DNA, 23 bp, synthetic construct
  source 1..23: mol_type = other DNA
ggcttccttatgttcaaacaggg

SEQ ID NO: 8 - DNA, 22 bp, synthetic construct
  source 1..22: mol_type = other DNA
gccgttactcgggtaaattaca

SEQ ID NO: 9 - DNA, 20 bp, synthetic construct
  source 1..20: mol_type = other DNA
ccagccacttctgagcatga

SEQ ID NO: 10 - DNA, 20 bp, synthetic construct
  source 1..20: mol_type = other DNA
cttctcttcccactcacggg

SEQ ID NO: 11 - DNA, 23 bp, synthetic construct
  source 1..23: mol_type = other DNA
acaatgaacgctacacactgcat

SEQ ID NO: 12 - DNA, 21 bp, synthetic construct
  source 1..21: mol_type = other DNA
tggcagtaacagccagaaaca

(19) United States
(12) Patent Application Publication
(10) Pub. No.: US 2025/0003986 A1
(43) Pub. Date: Jan. 2, 2025

(54) K17 AS A BIOMARKER FOR TUMOR RESPONSE TO IMMUNOTHERAPY

(71) Applicant: Wisconsin Alumni Research Foundation, Madison, WI (US)

(72) Inventors: Paul LAMBERT, Madison, WI (US); Wei WANG, Madison, WI (US)

(21) Appl. No.: 18/701,024

(22) PCT Filed: Oct. 14, 2022
(86) PCT No.: PCT/US2022/078135

§ 371 Date: Apr. 12, 2024

Related U.S. Application Data
(60) Provisional application No. 63/255,851, filed on Oct. 14, 2021.
(60) Provisional application No. 63/328,705, filed on Apr. 7, 2022.

(51) Int. Cl. G01N 33/68

(52) U.S. Cl. CPC G01N 33/6893; G01N 33/6872; G01N 2333/4742; G01N 2333/70532

(57) ABSTRACT

The present invention provides methods of detecting responsiveness of cancers to immunotherapies and determining which treatment to use with a particular cancer. The methods include obtaining a sample from the subject and detecting the expression level of keratin 17 (K17) in the sample. Low levels of K17 expression indicates that the cancer is responsive to the immunotherapy and high levels of K17 expression indicate that the cancer is not responsive to immunotherapy and that other methods of treating the cancer should be used.

### CROSS-REFERENCE TO RELATED APPLICATIONS

This application claims priority to U.S. Provisional Application No. 63/255,851 filed on Oct. 14, 2021, and U.S. Provisional Application No. 63/328,705 filed on Apr. 7, 2022, the contents of both of which are incorporated by reference in their entirety.

### STATEMENT REGARDING FEDERALLY SPONSORED RESEARCH

This invention was made with government support under CA022443, CA210807 and DE026787 awarded by the National Institutes of Health. The government has certain rights in the invention.

### SEQUENCE LISTING

A Sequence Listing accompanies this application and is submitted as an XML file of the sequence listing named “960296.04333_ST26.xml” which is 11,472 bytes in size and was created on Sep. 27, 2022. The sequence listing is electronically submitted via EFS-Web with the application and is incorporated herein by reference in its entirety.

### BACKGROUND

Stress keratin 17 (K17) is a stress-induced keratin expressed in epithelial cells during wound healing, inflammation and autoimmune diseases (1-4). In normal healthy epithelium, expression of K17 is limited to the medulla compartment of the hair and skin appendages (4, 5). K17 is overexpressed in a variety of cancer types, including cancers of the skin, cervix, breast, ovary and the head and neck region, and is associated with poor prognosis in breast, oropharyngeal and ovarian cancers (6-11). How K17 contributes to a worse prognosis in cancer patients is unclear. Disruption of the K17 gene in Human Papillomavirus (HPV) transgenic mice and in Gli2 transgenic mice suppressed cervical and skin carcinogenesis, respectively, and led to a differential cytokine expression profile suggesting a role of K17 in host immunity (6, 12). Our prior work using mouse papillomavirus (MmuPV1) as a model to study cellular immune response to papillomavirus-induced neoplastic disease indicated that K17 overexpression contributes to persistent viral infection and papillomatosis by downregulating T cell infiltration (13). We also observed an inverse correlation between the level of K17 expression and the expression of CD8a and IFNγ-related genes at the RNA level when we interrogated head and neck squamous cell carcinoma (HNSCC) tumor RNA-Seq data from the Cancer Genome Atlas (TCGA) (13). K17 overexpression has been reported in a wide range of cancer types, including ones that are not associated with any known viruses (9, 11), and it was found to contribute to carcinogenesis in non-viral induced Gli2 transgenic mice (6).

Currently, two anti-PD1 immune checkpoint blocking antibodies, nivolumab and pembrolizumab, are FDA-approved to treat recurrent squamous cell carcinoma of the head and neck region, albeit with a response rate of less than 20% (14). High T cell infiltration in a patient's tumor is associated with better response to immune checkpoint therapy (15). Accordingly, there remains a need in the art for better method of detecting which cancers can be treated and are responsive to immune checkpoint therapies.

### SUMMARY

In a first aspect, the disclosure provides a method of determining responsiveness of a cancer to immunotherapy in a subject, the method comprising: (a) obtaining a sample from the subject; and (b) detecting the expression level of keratin 17 (K17) in the sample. A low level of K17 expression indicates that the cancer is responsive to the immunotherapy.

In another aspect, the disclosure provides a method of determining responsiveness of a cancer to immunotherapy in a subject. The method comprising: (a) obtaining a sample from the subject; (b) detecting the expression level of keratin 17 (K17); and (c) detecting the expression level of at least one additional marker in the sample. When the additional marker is selected from CCL15, CCL23, CCL7, CXCL9, ENTPD1, SIRPG, ADORA2A, CCR1, CD47, CXCR3, DPP4, and FCGR2A and a low level of K17 expression and the additional marker or combination of paired receptor-ligand paired additional markers are detected then the cancer is responsive to the immunotherapy. When the additional marker is selected from IFNγ, CXCL9, CXCL10, CXCL11, PD-L1, CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, and SPN and a low level of K17 expression and a low level of the additional marker expression or combination of paired receptor-ligand paired additional markers are detected then the cancer is responsive to the immunotherapy.

In a further aspect, the disclosure provides a method of predicting if a cancer is non-responsive to an immunotherapy, the method comprising: (a) obtaining a sample from a subject; and (b) detecting the expression level in the sample of keratin 17 (K17). Detection of a high level of expression of K17 indicates that the cancer is non-responsive to immunotherapy.

In yet another aspect, the disclosure provides a method of determining if a cancer is non-responsive to immunotherapy in a subject. The method comprises: (a) obtaining a sample from the subject; (b) detecting the expression level of keratin 17 (K17); and (c) detecting the expression level of at least one additional marker in the sample. When the additional marker is selected from CCL15, CCL23, CCL7, CXCL9, ENTPD1, SIRPG, ADORA2A, CCR1, CD47, CXCR3, DPP4, and FCGR2A and a high level of K17 expression and a low level of the additional marker expression or combination of paired receptor-ligand paired additional markers are detected then the cancer is non-responsive to the immunotherapy. When the additional marker is selected from IFNγ, CXCL9, CXCL10, CXCL11, PD-L1, CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, and SPN and a high level of K17 expression and a high level of the additional marker expression or combination of paired receptor-ligand paired additional markers are detected then the cancer is non-responsive to the immunotherapy.

Further aspects are described herein.

### BRIEF DESCRIPTION OF THE DRAWINGS

FIG. 1. Human head and neck cancer samples with high K17 expression present low level of CD8+ cell infiltration in tumors. A) Examples of TMA images analyzed for K17 expression level and CD8% positivity in tumors (E-Cad+). Left column shows fluorescent views of one K17 high tissue and one K17 low tissue, with K17 staining shown in red. Right column shows single color view of E-Cad staining and CD8 staining (orange pseudo color) of the same tissues shown on left side. Far right column shows enlarged image of CD8 staining. B) (left) K17 mean fluorescence intensity (X-axis) and CD8% positivity (Y-axis) in E-cadherin marked tumor regions. Spearman correlation was used. (middle) K17 MFI in p16+ versus p16− samples. Student t-test was used (****p<0.001). (right) % CD8 positivity in tumor for p16+ versus p16− samples. Student t-test was used. C) K17 MFI of triplicate primary cancer samples are averaged for the same patient. Log rank test was used to compare survival times between patients in the top 25% K17 MFI range and patients in the bottom 25% K17 MFI range. D) Log rank test was used to compare (left) patients that had the most 25% K17 MFI and most 25% CD8 positivity versus patients that had the most 25% K17 MFI and least 25% CD8 positivity; (right) patients that had the least 25% K17 MFI and most 25% CD8 positivity versus patients that had the least 25% K17 MFI and the least 25% CD8 positivity.

FIG. 2. Stress keratin 17 supports syngeneic head and neck cancer cell growth in immunocompetent mice. A) Immunofluorescent staining of in vitro cultured MOC2 cells and 3 K17-KO variants (10×). Green: K17; Blue: DAPI. MOC2: parental MOC2 cells; K17KO MOC2-1: K17KO MOC2 cells CRISPER/Cas9-deleted with gRNA targeting Exon1 of K17 selected in puromycin; K17KO MOC2-2: K17KO MOC2 cells CRISPER/Cas9-deleted with gRNA targeting Exon4 of K17 selected in puromycin; K17KO MOC2-3: K17KO MOC2 cells CRISPER/Cas9-deleted with gRNA targeting Exon5 of K17 selected in puromycin. B) Western blot of K17 and beta-actin in proteins collected from MOC2, K17KO-2 and K17KO-3 cell lines. C) Relative expression of K17 mRNA measured by qRT-PCR in K17KO MOC2 cell lines in vitro, normalized to GAPDH mRNA level in the parental MOC2 cells. Standard errors are shown. D) NSG mice were subcutaneously injected with 3×10⁵ MOC2 parental cells (n=8 NSG-MOC2), or K17KO MOC2-1 cells (n=7 NSG-K17KO-1), or K17KO MOC2-2 cells (n=8 NSG-K17KO-2), or K17KO MOC2-3 cells (n=8 NSG-K17KO-3). Tumor sizes were monitored twice weekly. Two-way ANOVA was used to compare tumor size between MOC2 and K17KO MOC2 tumors for each time point; *p<0.05, ns=not significant for any time point (top); Mouse death were recorded or mice were euthanized when tumor reached 15 mm in diameter. Log rank was used to compare survival times between groups. E) C57BL/6 mice were subcutaneously injected with 3×10⁵ MOC2 parental cells (n=5 BL6-MOC2), or K17KO MOC2-1 cells (n=5 BL6-K17KO-1), or K17KO MOC2-2 cells (n=5 BL6-K17KO-2), or K17KO MOC2-3 cells (n=6 BL6-K17KO-3). Tumor sizes were monitored until day 28 post injection. Two-way ANOVA was used to compare tumor size between MOC2 and K17KO MOC2 tumors for each time point; *p<0.05 for day 28 tumor size (top); Representative images of visible tumors from MOC2 tumor and remaining K17KO MOC2 tumor on day 28 post injection (bottom). F) Number of mice that completely rejected tumors in C57BL/6 on day 28 post injection. Fisher's exact test was used to compare MOC2 tumor and K17KO MOC2 tumor rejections; *p<0.05. G) C57BL/6 mice were subcutaneously injected with 2×10⁵ MOC2 parental cells or K17KO MOC2-1 cells or K17KO MOC2-2 cells. Tumors were collected between day 28 to day 31 post injection for flow cytometry of tumor infiltrating CD45, CD4 and CD8 cells. The frequency of CD45, CD4 and CD8 in total live cells were pooled for K17KO MOC2-1 and K17KO MOC2-2 tumors (K17KO MOC2). The data were pooled from two independent repeat experiments. Unpaired t-test was used to compare frequencies in MOC2 tumors versus K17KO MOC2 tumors; **p<0.01, ****p<0.0001. Standard errors are shown. H) Immunofluorescent staining of CD8 (green), K14 (red, keratinocyte/tumor cell marker) and DAPI (blue) in MOC2 tumor versus K17KO MOC2 tumor (20×). I) Heatmap of differentially expressed genes between K17KO MOC2 tumors versus MOC2 tumors. J) Normalized enrichment score of top seven upregulated and top seven downregulated hallmark signaling pathways comparing K17KO MOC2 tumors versus MOC2 tumors, using GSEA analysis. K) Relative mRNA expression of IFNg, CXCL9, CXCL10, CXCL11 and PD-L1 in K17KO MOC2 tumors compared to MOC2 tumors, normalized to GAPDH mRNA level, measured by qRT-PCR of bulk tumor RNA.

FIG. 3. T cells are required for rejection of K17KO MOC2 tumors in C56BL/6 mice. A) C57BL/6 mice were subcutaneously injected with 2×10⁵ K17KO MOC2-2 cells. Mice were depleted for CD4 and CD8 cells starting three days post injection, throughout the study. Tumor sizes were measured on day 11 and day 18 post injection. Left: Two-way ANOVA was used to compare average tumor size at each time point, **p<0.01, standard error is shown. Right: Fisher's exact test was used to compare between two groups for number of mice that completely reject tumor (tumor-free) and number of mice that did not reject tumor (tumor). B) Three K17KO MOC2-2 tumors from T cell depleted mice or isotype control mice were collected on day 18 post injection for flow cytometry analysis of tumor-infiltrating CD4 and CD8 cells. Both CD4 and CD8 cells were confirmed depleted from tumors growing in T cell-depleted mice. C) A single cell clone of K17KO MOC2-3 cells (K17KOMOC2) or parental MOC2 were injected subcutaneously at 2×10⁵ cells into C57BL/6 mice. Mice bearing K17KOMOC2 tumors received anti-CXCR3 or isotype control antibody starting one day before tumor injection, throughout the study. Tumor sizes were monitored twice every week. Two-way ANOVA was used to compare average tumor size for each time. *p<0.05 for day 18 time point. ***p<0.001 for day 18 time point. Standard errors are shown. D) Probability of mice with tumors that persisted (failed to completely regress) in anti-CXCR3 treated mice versus isotype control mice. Gehan-Breslow-Wilcoxon test was used to compare the regression line between two groups. *p<0.05.

FIG. 4. Stress keratin 17 knockout tumors have improved response to immune checkpoint blockade therapy. C57BL/6 mice were injected with 3×10⁵ K17KO MOC2-2 cells or parental MOC2 cells. Mice bearing a K17KO MOC2 tumor that was larger than 2 mm×2 mm on day 14 post injection and mice bearing MOC2 tumors were subject to anti-CTLA4+anti-PD1 treatment starting on day 14 post injection, for up to four doses (indicated by arrows). Tumor sizes were monitored. CR: complete responder were mice that completely rejected the tumor by day 45 post injection. A) Individual (on left) and average (on right) K17KOMOC2 tumor growth are shown. B) Individual and average MOC2 tumor growth treated at the same time. C) Individual and average MOC2 tumor growth treated at the same size (starting at day 6 post injection for the MOC2 tumors) as the >2×2 day 14-K17KOMOC2 tumors in FIG. 4A. Two-way ANOVA was used to compare average tumor size between groups for each time point measured. ns: not significant, ****p<0.0001. D) Mice that spontaneously rejected K17KO MOC2 tumors (K17KO-immunized) and mice that completely rejected K17KO MOC2 tumors upon ICB therapy (K17KO-cured) were subcutaneously re-challenged with 150,000 MOC2 parental cells, along with some naïve control mice. Average MOC2 tumor growth is shown. Two-way ANOVA was used to compare average tumor size between groups for each time point measured. ns: not significant, *p<0.05 for day 20. Standard errors are shown. E) Percent of mice that were able to reject MOC2 tumors (tumor-free) and percent of mice that did not reject MOC2 tumors (tumor). Fisher's exact test used to compare between each group. ns: not significant, *p<0.05. F) Spleens from all mice in FIG. 4D were collected on day 20 post MOC2 injection. Spleen cells were subject to flow cytometry analyses for effector memory (eff memory) and central memory defined by CD44 and CD62L staining. One-way ANOVA was used to compare the frequency of memory cells in total live spleen cells between each group. ns: not significant, *p<0.05, **p<0.01, ***p<0.001. G) Spleen cells from K17KO-immunized mice and K17KO-cured mice that were challenged with MOC2 cells in FIG. 4D were subject to CD11b and Gr1 analyses by flow cytometry. Student t-test was used to compare cell frequency between mice that rejected MOC2 tumors (tumor free) versus mice that did not reject MOC2 tumors (tumor-bearing). *p<0.05.

FIG. 5. High level of K17 expression in head and neck cancer is associated with poor response to anti-PD1 immunotherapy. A) Two representative images for K17 high and K17 low tissue (20×). B) Association analysis between pretreatment tumor K17 expression and clinical response was based on a high expression cut-off of >5% strong cytoplasmic staining in tumor cells. Fisher's exact test was used. C) Kaplan-Meier estimates of overall survival. Log rank test was used. D) Kaplan-Meier estimates of progression-free survival (PFS). Log rank test was used.

FIG. 6. K17KO MOC2 tumors had switched tumor immune microenvironment phenotypes. A) UMAP (Uniform Manifold Approximation and Projection) high-dimensional deductional map of 21,894 CD45+ cells between MOC2 and K17KOMOC2 tumors (2 replicates each group) and the annotation of major immune cell-types: T cells (CD4/CD8/Treg), NKs, Mast cells, B cells, Neutrophils, Monocytes/Macrophages/DCs. B) Representative markers' expression of major immune cell-types, color presented scaled average expression levels, circle size presents the percentage of cells that expressed specific markers. C) Frequencies of major immune-cell types (out of CD45+ cells) and the ratio of CD4/CD8, CD4/Treg, CD8/Treg between K17KOMOC2 and MOC2 tumors. D) UMAP of 6,737 myeloid cells (excluding neutrophils) including different DCs and Macrophage subsets; the macrophage subset were named using the representative marker in each subset (Supplementary FIG. 9). E) Frequencies of 9 DC and macrophage subsets (out of number of CD45+ cells) between K17KO vs MOC2.

FIG. 7. A) Representative images excluded for analyses. B) Correlation analysis of TCGA HPV+ and HPV− HNSCC patient CD8 and K17 RNA expression level. Spearman correlation was used. C) Kaplan-Meier estimates of overall survival based on 5%, 10% and 25% cut off in K17 expression. Log rank test was used.

FIG. 8. A) A representative image from immunofluorescence staining of K14 (MOC2 tumor cell marker, red), K17 (green) and DAPI (blue) showed outgrowth of K17+ cells in vivo from K17KO MOC2-2 bulk population injected into C57BL/6 mice (10×). B) Western blot for K17 and beta-actin in protein collected from single cell clone 5 of K17KO MOC2-3 cells. C) 3×10\hat{ }5 of single cell clone 5 were injected into NSG or C57BL/6 mice. Tumor volume were measured on day 14 post injection. D) 3×10\hat{ }5 K17KO MOC2 cells or parental MOC2 cells were injected into Cas9 knock-in mice (n=2 K17KO MOC2, n=2 MOC2) or wildtype C57BL/6 mice (n=4 K17KO MOC2, n=3 MOC2). Tumor growth were followed for 18 days. E) K17KO MOC2 tumors have delayed growth in Cas9 knock-in mice. Student t-test was used to compared tumor volume at day 18. F) 3×10\hat{ }5 K17KO MOC2-2 cells or MOC2 cells expressing Cas9 were injected into C57BL/6 mice and their tumor growth were followed for 18 days. Student t-test was used to compared tumor volume at day 18. G) Gating strategy for tumor-infiltration CD4+ and CD8+ cells.

FIG. 9. Gating strategy for tumor-infiltrating CD4 and CD8 analyses in FIG. 3B.

FIG. 10. K17KO MOC2 cell-immunized mice can generate protective memory response against parental MOC2 cells. C57BL/6 mice that spontaneously cleared K17KO MOC2 tumors (K17KOMOC2 immunized) or naïve C57BL/6 mice (naïve) were challenged with 2×10⁵ parental MOC2 cells subcutaneously. MOC2 tumor growth were monitored until day 22 post injection. Spleens were collected from all mice for flow cytometry analyses on day 22 post MOC2 cell challenge. A) Left: number of mice that completely rejected MOC2 tumors (tumor-free) and number of mice that did not reject MOC2 tumors (tumor) are shown. Fisher's exact test was used to compare between two groups. **p<0.01. Right: average MOC2 tumor growth. Two-way ANOVA was used to compare tumor size for each timepoint. ns: not significant for any time point. Data are pooled from three independent repeat experiments. B) Spleen cells were subjected for flow cytometry analyses for CD44 and CD62L expression on pre-gated CD45+CD4+ cells and CD45+CD8+ cells. C) Percentage of memory cell subsets in total live spleen cells defined by gates shown in figure B. Student t-test was used to compare the frequency of memory cells in K17KOMOC2 immunized mice versus naïve mice that were challenged with parental MOC2 cells in figure A. *p<0.05, **p<0.01, ***p<0.001. Standard errors are shown. Data are pooled from three independent repeat experiments.

FIG. 11. Gating strategy for CD11b+Gr1 high and CD11b+Gr1 low cells in FIG. 4G.

FIG. 12. Representative staining patterns of K17 by immunohistochemistry. K17 high staining patterns (20×): diffuse strong (3+) cytoplasmic staining; strong (3+) cytoplasmic staining in lower percentage of tumors with mixed keratinizing and basaloid morphology. K17low staining patterns: absence of K17 expression (20×); Basal type staining (20×); Perinuclear/golgi type staining (40×, bottom).

FIG. 13. NK cells in MOC2 tumors have decreased expression of maturation markers. A) UMAP presentation of 1,040 NK cells between MOC2 and K17KO MOC2 tumors (2 replicates each group) including two subsets: mature NK cells, and immature NK cells. B) Dotplot of the 10 differentially expressed genes NK cell subsets. C) Frequencies of NK cell subsets (out of the total NK cell population) between K17KO MOC2 and MOC2 tumors.

FIG. 14. Average expression of PD-L1/2, CXCL9/10 and CXCR3 in different immune cell-types K17KOMOC2 vs MOC2. Z-score showed higher expression of PD-L1 (CD274), PDL2 (Pdcd1lg2) as well as CXCL9/10 in myeloid cells (macrophages/DC and neutrophils), and CXCR3 in CD8 T cells (red box).

FIG. 15. Top 10 DE genes of Mon/Mac/DC subsets. The differentially expressed genes (top 10 for each subset in the comparison of 1 subset vs the rest) was identified using MAST (see Methods) with cutoff average log 2FC of 0.25 and FDR-corrected p value<0.01. The heatmap represents expression of top 10 genes across down-sampled representative of the cells across different myeloid subsets.

FIG. 16. 1000 U of mouse IFNγ were added onto K17KO MOC2 and MOC2 cells in tissue culture overnight. Cells were collected and stained for K17 and CXCL9 for flow cytometry analysis. A) CXCL9+ cell gating on K17KO MOC2 and MOC2 cells. B) Percent of CXCL9+ cells in K17KO MOC2 and MOC2 cells. Student t-test was used. ****p<0.001

FIG. 17. Ligand: Receptor (LR) interaction and Transcription Factor network analysis. A) Number of statistically significant LR interactions inferred by CellPhoneDB (p<0.05) that are shared and unique between MOC2 vs K17KOMOC2. B) Expression violin plots showing levels of expression of CXCL9 in different immune cell-types and its receptor CXCR3 in other immune cell types between K17KOMOC2 vs MOC2. C) Regulons (named by master TFs) that are differentially expressed in different immune cell-types between K17KOMOC2 vs MOC2 using t-values from generalized linear model.

### DETAILED DESCRIPTION

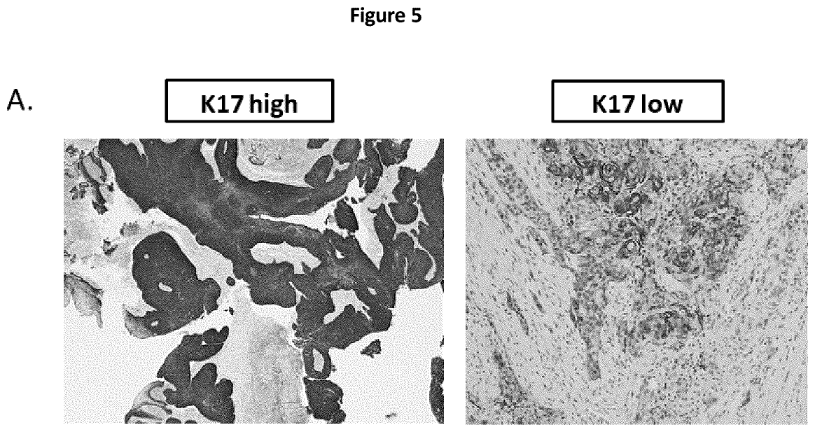

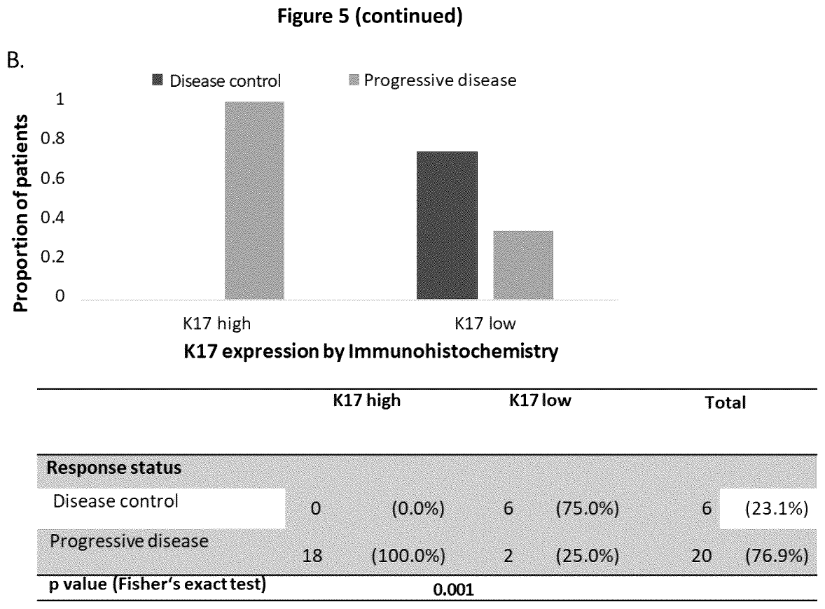

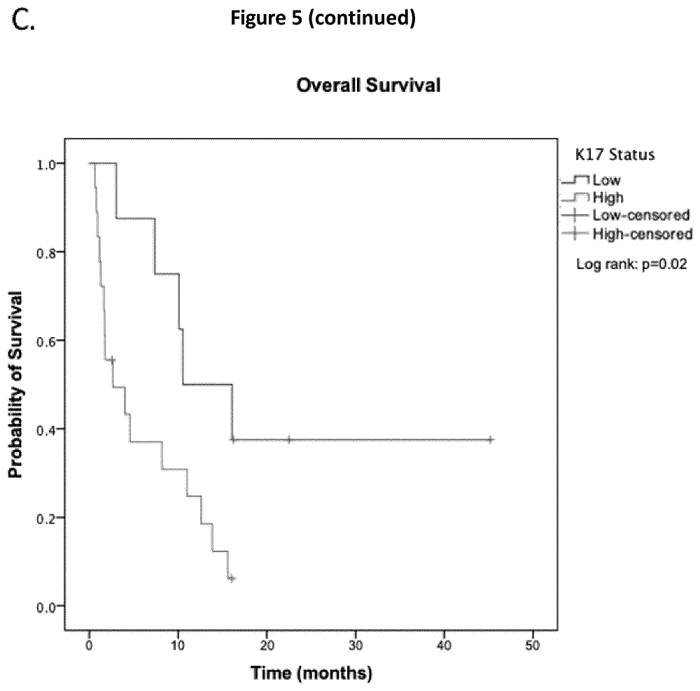

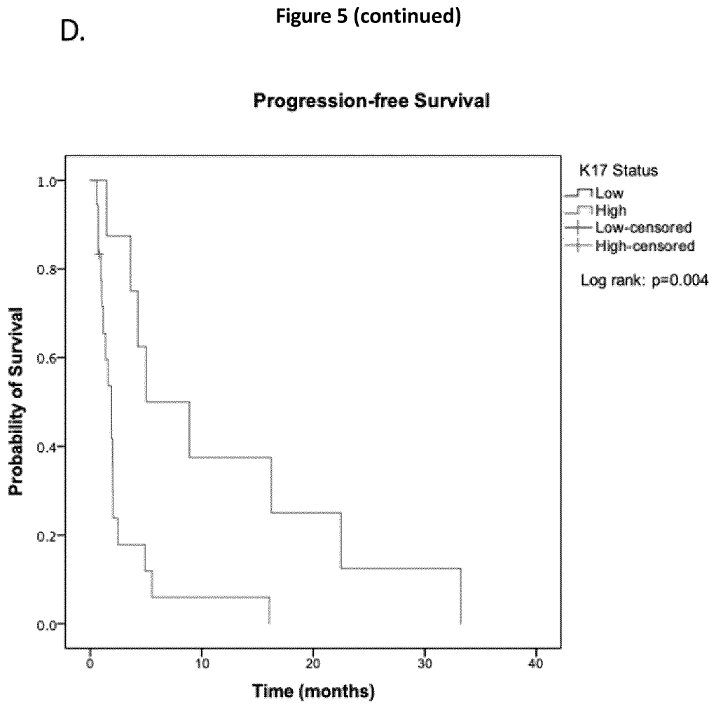

The present invention is based on work by the inventors demonstrating a link between K17 expression and CD8+ T cells via RNA expression level by analysis of head and neck cancer (HNC) tissue microarray (TMA) for K17 and CD8 protein levels. A syngeneic mouse oral cancer line, MOC2, derived from a chemical carcinogen-induced oral cavity tumor arising in C57BL/6 mice (16) was used to demonstrate how K17 mediates immune evasion of HNSCC in vivo. MOC2 cells, when injected subcutaneously into syngeneic immunocompetent C57BL/6 mice, form fast growing tumors that are immunologically “cold”, with limited T cell infiltration and low predicted neoantigen levels, and are resistant to combined immune checkpoint blockade treatment (anti-CTLA4+anti-PD1) (17, 18). When the inventors knocked out K17 in MOC2 cells, they turned MOC2 tumors into immunologically “hot” tumors with increased T cell infiltration and activated T cell gene expression, as well as enhanced response to anti-CTLA4+anti-PD1 treatment. The data demonstrate that K17 contributes to immune evasion and to resistance to checkpoint blockade therapy in HNSCC, and that K17 is a strong predictive biomarker for HNSCC patients whose tumors are resistant to immune checkpoint blockade therapy. Further, as demonstrated in the examples, high levels of K17 in tumor samples from head and neck cancer indicated a poor response to immunotherapy (see, e.g., Example 1 and FIG. 5).

In one embodiment, the present disclosure provides a method of determining responsiveness of a cancer to immunotherapy in a subject, the method comprising: (a) obtaining a sample from the subject; and (b) detecting the expression level of keratin 17 (K17) in the biological sample. A low level of K17 expression indicates that the cancer is responsive to the immunotherapy.

In another embodiment, the method includes (a) obtaining a sample from the subject; and (b) detecting the expression level of keratin 17 (K17) in the sample; and (c) selecting a patient that has a high level of K17 expression. The high level of K17 expression indicates that the cancer is not responsive to the immunotherapy. In further embodiments, the method comprises treating the subject with a cancer therapy which is not an immunotherapy such as radiation therapy or chemotherapy.

Stress keratin 17 or keratin 17 (K17) is a stress-induced keratin expressed in epithelial cells during wound healing, inflammation and autoimmune diseases. In normal healthy epithelium, expression of K17 is limited to the medulla compartment of the hair and skin appendages. K17 is overexpressed in a variety of cancer types, including cancers of the skin, cervix, breast, ovary and the head and neck region. Keratins are a group of tough, fibrous proteins that form the structural framework of certain cells, particularly cells that make up the skin, hair, nails, and similar tissues.

Responsiveness to cancer therapy (specifically immunotherapy) refers to the ability of an agent to reduce, slow or inhibit cancer cell growth and spread, e.g. the ability of the immunotherapy (e.g., immune checkpoint inhibitor) to reduce, slow or inhibit cancer cell growth and spread. Non-responsiveness to cancer therapy (specifically immunotherapy) refers to the inability of an agent to reduce, slow or inhibit cancer cell growth and spread, e.g. the inability of the immunotherapy (e.g., immune checkpoint inhibitor) to reduce, slow or inhibit cancer cell growth and spread.

As used herein, the term immunotherapy refers to a biological therapy for cancer treatment that helps and improves the ability of the immune system to fight cancer. Suitably, the immunotherapies described herein are immune checkpoint inhibitors. As used herein, “immune checkpoint therapy” (“ICT”) refers to an intervention that is targeted to interfere with the normal function of “immune checkpoints.” In some embodiments, ICT comprises a treatment that interferes with the function of PD-1 or its ligands PD-L1 and PD-L2. In another embodiment, the immune checkpoint inhibitors are agents capable of blockade of T cell immune checkpoint receptors, including but not limited to PD-1, PD-L1, TIM-3, LAG-3, CTLA-4, and CSF-1R and combinations of such checkpoint inhibitors. In some embodiments, the immune checkpoint inhibitors include anti-PD-1 antibody, anti-PD-L1 antibody, anti-CTLA4 antibody, anti-LAG-3 antibody, and/or anti-TIM-3 antibody. In some embodiments, the ICT comprises a monoclonal antibody targeted to PD-1. In some embodiments, the monoclonal ICT therapy is selected from the group consisting of pembrolizumab, nivolumab, cemiplimab, atezolizumab, dostarlimab, durvalimab, and avelumab.

Checkpoint inhibitors that comprise anti-PD1 antibodies or anti-PD-L1-antibodies or fragments thereof are known to those skilled in the art, and include, but are not limited to, cemiplimab, nivolumab, pembrolizumab, MEDI0680 (AMP-514), spartalizumab, camrelizumab, sintilimab, toripalimab, dostarlimab, and AMP-224. Checkpoint inhibitors that comprise anti-PD-L1 antibodies known to those skilled in the art include, but are not limited to, atezolizumab, avelumab, durvalumab, and KN035. The antibody may comprise a monoclonal antibody (mAb), chimeric antibody, antibody fragment, single chain, or other antibody variant construct, as known to those skilled in the art. PD-1 inhibitors may include, but are not limited to, for example, PD-1 and PD-L1 antibodies or fragments thereof, including, nivolumab, an anti-PD-1 antibody, available from Bristol-Myers Squibb Co and described in U.S. Pat. Nos. 7,595,048, 8,728,474, 9,073,994, 9,067,999, 8,008,449 and 8,779,105; pembrolizumab, an anti-PD-1 antibody, available from Merck and Co and described in U.S. Pat. Nos. 8,952,136, 83,545,509, 8,900,587 and EP2170959; atezolizumab is an anti-PD-L1 available from Genentech, Inc. (Roche) and described in U.S. Pat. No. 8,217,149; avelumab (Bavencio, Pfizer, formulation described in PCT Publ. WO2017097407), durvalumab (Imfinzi, Medimmune/AstraZeneca, WO2011066389), cemiplimab (Libtayo, Regeneron Pharmaceuticals Inc., Sanofi, see, e.g., U.S. Pat. Nos. 9,938,345 and 9,987,500), spartalizumab (PDR001, Novartis), camrelizumab (AiRuiKa, Hengrui Medicine Co.), sintillimab (Tyvyt, Innovent Biologics/Eli Lilly), KN035 (Envafolimab, Tracon Pharmaceuticals, see, e.g., WO2017020801A1); tislelizumab available from BeiGene and described in U.S. Pat. No. 8,735,553; among others. Other PD-1 and PD-L1 antibodies that are in development may also be used in the practice of the present invention, including, for example, PD-1 inhibitors including toripalimab (JS-001, Shanghai Junshi Biosciences), dostarlimab (GlaxoSmithKline), INCMGA00012 (Incyte, MarcoGenics), AMP-224 (AstraZeneca/MedImmune and GlaxoSmithKline), AMP-514 (AstraZeneca), and PD-L1 inhibitors including AUNP12 (Aurigene and Laboratoires), CA-170 (Aurigen/Curis), and BMS-986189 (Bristol-Myers Squibb), among others (the references citations regarding the antibodies noted above are incorporated by reference in their entireties with respect to the antibodies, their structure and sequences). Fragments of PD-1 or PD-L1 antibodies include those fragments of the antibodies that retain their function in binding PD-1 or PD-L1 as known in the art, for example, as described in AU2008266951 and Nigam et al. “Development of high affinity engineered antibody fragments targeting PD-L1 for immunoPED,” J Nucl Med May 1, 2018 vol. 59 no. supplement 1 1101, the contents of which are incorporated by reference in their entireties.

Checkpoint inhibitors that comprise anti-CTLA4 antibodies or fragments thereof are known to those skilled in the art, and include, but are not limited to, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, belatacept, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP1212422B1. Additional CTLA4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA4 antibodies that can be used in a method of the present invention include, for example, those disclosed in: WO 98/42752; U.S. Pat. Nos. 5,977,318, 6,207,156, 6,682,736, 7,109,003, and 7,132,281; Hurwitz 1998; Camacho 2004 (antibody CP-675206); and Mokyr 1998. In some preferred embodiments, the anti-CTLA4 antibody is selected from the group consisting of ipilimumab and tremelimumab.

Additional CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway, antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway. In some implementations, the CTLA4 antagonist may be an anti-B7-1 antibody, an anti-B7-2 antibody, an anti-B7-H4 antibody.

The subject or patient described herein is mammal, preferably a human having or suspected of having cancer. The terms “cancer” or “tumor” mean any abnormal proliferation or uncontrolled growth of cells, including solid tumors, and may spread to other locations in the organism (e.g., metastasize). Suitably, the cancer is an epithelial originated cancer. For example, but not limited to, the cancer can be head and neck cancer, skin cancer, small cell lung cancer, cervical cancer, lung squamous cell carcinoma, breast, pancreatic cancer, among others.

As used herein, “sample”, “biological sample” or “test sample” refers to any sample of tissue, fluid, or material derived from a living organism. In some embodiments, the living organism is a primate. In some embodiments, the living organism is a human being, or Homo sapiens. Exemplary biological samples include, but are not limited to, a tumor tissue sample, for example, a biopsy sample, a blood sample, or a sample from an excised tumor.

In another embodiment, the biological sample may be a blood sample to test for tumor specific CD8+ T cells.

As described more in the Examples, tissue samples from cancer, specifically head and neck cancer TMA, from a subset of patients were found to have significantly higher K17 expression (demonstrated by RNA expression), and the levels of CD8 infiltrating cells were low in the patients with high K17 expression. K17 expression in some cancers facilitates evasion of immune surveillance and resistance to ICB therapy as shown in the Examples. Thus, in some embodiments, the ability to determine a high K17 expression level in a sample from cancer in a patient indicates that the patient is not (will not be) responsive to immunotherapy.

Suitable methods of determining the expression of K17 in a sample are known and understood in the art. In one embodiment, the expression is measured by nucleic acid expression, e.g., gene expression or mRNA expression. In another embodiment, the expression is measured by K17 protein expression in the sample. Suitable methods and reagents for these methods are known in the art. For example, suitable methods to measure expression levels of DNA/RNA, include, but are not limited to, Northern blot analysis, nuclease protection assays (NPA), in situ hybridization, reverse transcription-polymerase chain reaction (RT-PCR), qRT-PCR, RNA-Seq, among others. Suitable methods to measure protein levels include, for example, immunohistochemistry, immunofluorescence, flow cytometry, mass spectroscopy, enzyme-linked immunosorbent assays (ELISA), quantitative ELISA, Western blotting and dot blotting, among others.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc.

The control as described herein refers to a sample from a normal tissue (e.g., a non-cancerous sample) or can refer to a responsive or non-responsive control cancerous tissue to which the sample from a subject can be compared. Control can also refer to a standard control that determines a baseline expression level to which the samples may be compared. The normal tissue may be derived from the subject with cancer or from a healthy subject. The control can also be an established level of expression based off healthy or unhealthy population statistics, for example. Samples with low expression of K17 similar to responsive controls are likely similarly responsive to immunotherapies and samples with high K17 expression similar to non-responsive cancerous controls are likely non-responsive to immunotherapies.

As described in the Examples, in cancer cells, high K17 expression was associated with low CD8 infiltration. Not to be bound by any theory, but it is thought that high K17 expression was associated with low tumor-specific T cell response to the tumor, and thus increased resistance of the tumor to immunotherapies that rely on T cell clearance, including CD8+ T cell clearance.

The method described herein for detecting the responsiveness to cancer in some embodiments further comprises, when low levels of K17 are detected, selecting the subject with low levels of K17 as having a cancer responsive to immunotherapy and treating the subject with cancer with an immunotherapy. Suitable immunotherapies include immune checkpoint inhibitors, including those described herein.

In another embodiment, the method for detecting the responsiveness of the cancer to immunotherapies comprises detecting a high level of K17 and selecting the subject having high levels of expression of K17 as being non-responsive to immunotherapy. In some further embodiments, the subject is further treated with a cancer therapy that is not an immunotherapy.

As used herein, “detecting” is defined as identifying the presence of a particular molecules within a sample. In some embodiments, detecting is performed by human observation. In some embodiments, detecting is performed by an automated device according to an established algorithm and involves no direct application of human observation or thought.

As used herein, “low level” and “high level” can refer to the relative or absolute level of expression of a particular gene, protein, or characteristic. Relative level of expression can be determined by comparing levels to a control. Relative RNA expression to a control can be described in terms of fold change, fold increase, or fold decrease. For example, a sample can have a 300-fold, 3,000-fold, or 10,000-fold decrease in K17 RNA expression compared to a non-responsive control and such a decrease in RNA expression of K17 is indicative of responsiveness to the immunotherapeutic. Absolute level of expression can also be determined by protein expression. For example, a low level of K17 protein expression can be less than or equal to 5%, 10%, or 25% of cells in a sample expressing K17. Another example can include a high level of K17 protein expression can be greater than 5%, 10%, or 25% of cells in a sample expressing K17. The ability to detect high or low levels of K17 in a sample, including a tumor sample, allows for a patient to be selected for treatment based on the ability to sort the subjects into those whose tumors are responsive to immunotherapies or those whose tumors are not responsive to immunotherapies based on the low or high expression of K17 in the sample, preferably a tumor sample.

In some embodiments, an additional marker or combination of paired receptor-ligand paired additional markers may be used in addition to K17 for the ability to select a subject that has a tumor that is or is not responsive to immunotherapies. For example, the markers that are listed in Table 4 and Table 5, which are ligand-receptor interactions that are differentially regulated and may contribute to K17 based responsiveness of the cancer, can be used as additional markers with K17, and detection of the K17 and additional marker allows for the patients to be classified as having a tumor that is or is not responsive to immunotherapies. In some embodiments, one or more markers from Table 4 and 5 are also detected in addition to K17 to classify the tumor within a subject as being responsive or non-responsive to immunotherapy. In some embodiments, those markers include, but are not limited to, IFNγ, CXCL9, CXCL10, CXCL11, PD-L1, CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, SPN, CCL15, CCL23, CCL7, CXCL9, ENTPD1, SIRPG, ADORA2A, CCR1, CD47, CXCR3, DPP4, and/or FCGR2A. When the additional marker is selected from CCL15, CCL23, CCL7, CXCL9, ENTPD1, SIRPG, ADORA2A, CCR1, CD47, CXCR3, DPP4, or FCGR2A, then a low level of K17 in combination with expression of any one of these markers or combination of paired receptor-ligand paired additional markers is indicative of responsiveness of the cancer to immunotherapies and the opposite is indicative of non-responsiveness to an immunotherapy. When the additional marker is selected from IFNγ, CXCL9, CXCL10, CXCL11, PD-L1, CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, or SPN, then a high level of K17 in combination with expression of any one of these markers or combination of paired receptor-ligand paired additional markers is indicative of non-responsiveness of the cancer to immunotherapies and a low level is indicative of responsiveness to an immunotherapy.

As used herein, the term “biomarker” or “marker” refers to a biological molecule that is associated with a particular disease or condition, and/or is indicative of a particular cell type, cell state, tissue type, or tissue state. Suitable biomarkers include, for example, nucleic acids or proteins. Biomarkers can be used as part of a predictive, prognostic, or diagnostic process. For example, biomarkers may be used to predict the likelihood that a particular subject will respond to a particular therapeutic. In some cases, the mere presence (or absence) of a biomarker in a biological sample is indicative of a particular condition, whereas in other cases the biomarker is only indicative of a condition when it is present at a particular level or in a specific location within a biological sample. For example, in some cases a biomarker is a differentially expressed gene. In some embodiments, the biomarker is a therapeutic target.

For purposes of the present invention, “treating” or “treatment” describes the management and care of a subject for the purpose of combating the disease, condition, or disorder. Treating includes the administration of a therapy described herein when it is determined that the subject would be provided a benefit by the administration of the treatment to prevent the onset of the symptoms or complications, alleviating the symptoms or complications, or eliminating the disease, condition, or disorder. The term “treating” can be characterized by one or more of the following: (a) the reducing, slowing or inhibiting the growth of cancer, including reducing slowing or inhibiting the growth of cancer cells; (b) preventing the further growth of tumors; (c) reducing or preventing the metastasis of cancer within a patient, and (d) reducing or ameliorating at least one symptom of the cancer. In some embodiments, the optimum effective amounts can be readily determined by one of ordinary skill in the art using routine experimentation.

In some embodiments, the immunotherapy is one or more immune checkpoint inhibitors, for example, one or more PD-1 inhibitors, PD-L1 inhibitors, TIM-3 inhibitors, LAG-3 inhibitors, CTLA-4 inhibitors, and CSF-1R inhibitors and combinations of such checkpoint inhibitors. In one embodiment, the immunotherapy may be a PD-1 inhibitor and a CTLA-4 inhibitor.

In some embodiments, the method further comprises as step (b) detecting the expression level of PD-L1 in addition to K17, wherein detection of high level of PD-L1 expression and low level of K17 expression indicates the tumor is responsive to the immunotherapy and may be able to select a subset of patients. In other embodiments, measuring the PD-L1 level is not included and does not provide additional benefits for selecting patients in addition to K17 demonstrating that K17 itself is a potent selector of tumors that are or are not responsive to immunotherapy.

In a further aspect, the method includes: (a) obtaining a sample from the subject; and (b) detecting the expression level of keratin 17 (K17) and PD-L1 in a sample; wherein a low level of K17 expression and a high level of PD-L1 expression indicated that the cancer is responsive to the immunotherapy. In a preferred example, the expression level is RNA expression level. In another embodiment, the expression level is protein expression level.

In another embodiment, the disclosure provides a method of predicting if a cancer is non-responsive to an immunotherapy. The method includes (a) obtaining a sample from a subject; and (b) detecting the expression level in the sample of keratin 17; wherein detection of a high level of expression of K17 predicts the cancer is non-responsive to immunotherapy. Preferably, the sample is a tumor sample or biopsy sample. In some embodiments, the method further comprises when high levels of K17 are detected: (c) treating the subject with cancer with a cancer therapy that is not an immune checkpoint inhibitor. Suitable cancer therapies that are not immunotherapies are known in the art. For example, suitable cancer therapies include, chemotherapeutics or radiation, among others. As used herein, “chemotherapeutics” refers to compounds used to treat cancer including, but not limited to, cytotoxic agents, targeted therapies, and hormonal therapies. This method may also be combined with the additional markers listed above.

In some embodiments, the cancer is head and neck cancer, skin cancer, small cell lung cancer, cervical cancer, lung squamous cell carcinoma, breast, pancreatic cancer, or other epithelial originated cancer, and the non-immunotherapy cancers therapies are therapies known and approved for these types of cancers.

In some embodiments, detecting expression of K17 is combined with the detection of the expression level of PD-L1, wherein detection of low level of PD-L1 expression and high level of K17 expression compared to a control indicates the tumor is non-responsive to the immunotherapy.

The methods described herein can be used to stratify cancer patients into different categories, e.g., responsive or non-responsive to immunotherapy (e.g., immune checkpoint therapy) and can be used by one to determine the best treatment option of that sub-stratified patient, thus allowing for the cancer therapies to have the best chance of a positive treatment outcome, extending patient life expectancy and positive outcome predictions, while avoiding unnecessary side effects and costs for treatments that are not likely to have a positive effect on patient outcome.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Stress Keratin 17 Expression in Head and Neck Cancer Contributes to Immune Evasion and Resistance to Immune-Checkpoint Blockade

This Example is directed to demonstrating in human head and neck squamous cell carcinoma (HNSCCs), high levels of expression of stress keratin 17 (K17), is associated with poor survival and resistance to immunotherapy. Experimental Design: We investigated the role of K17 in regulating both the tumor microenvironment and immune responsiveness of HNSCC using a syngeneic mouse HNSCC model, MOC2. MOC2 gives rise to immunologically cold tumors that are resistant to immune checkpoint blockade (ICB). We engineered multiple, independent K17 knockout (KO) MOC2 cell lines and monitored their growth and response to ICB. We also measured K17 expression in human HNSCC of patients undergoing ICB. Summary of Results: MOC2 tumors were found to express K17 at high levels. When knocked out for K17 (K17KO MOC2), these cells formed tumors that grew slowly or spontaneously regressed and had a high CD8+ T cell infiltrate in immunocompetent syngeneic C57/BL6 mice compared to parental MOC2 tumors. This phenotype was reversed when we depleted mice for T cells. Whereas parental MOC2 tumors were resistant to ICB treatment, K17KO MOC2 tumors that didn't spontaneously regress were eliminated upon ICB treatment. In a cohort of HNSCC patients receiving Pembrolizumab, high K17 expression correlated with poor response. Single cell RNA seq analysis revealed broad differences in the immune landscape of K17KO MOC2 tumors compared to parental MOC2 tumors, including differences in multiple lymphoid and myeloid cell types.

This Example demonstrates that K17 expression in HNSCC contributes to immune evasion and resistance to immune checkpoint blockade treatment by broadly altering immune landscapes of tumors. To test our hypotheses and validate the link between K17 expression and CD8+ T cells via RNA expression level, we analyzed a HNSCC tissue microarray (TMA) for K17 and CD8 protein levels, and correlated K17 expression level with patients' overall survival. To test how K17 mediates immune evasion of HNSCC in vivo, we utilized a syngeneic mouse HNSCC line, MOC2, derived from a chemical carcinogen-induced oral cavity tumor arising in C57BL/6 mice (16). MOC2 cells, when injected subcutaneously into syngeneic immunocompetent C57BL/6 mice, form fast growing tumors that are immunologically “cold”, with limited T cell infiltration and low predicted neoantigen levels, and are resistant to combined immune checkpoint blockade treatment (anti-CTLA4+anti-PD1) (17, 18). We investigated whether knocking out K17 can turn an immunologically cold tumor into an immunologically hot tumor and whether MOC2 tumors would have increased responsiveness to immune checkpoint blockade treatment in the absence of K17. To confirm our hypothesis with human patient data, we also evaluated the K17 expression level in a cohort of HNSCC patients who received Pembrolizumab. These studies demonstrate that K17 contributes to immune evasion and to resistance to checkpoint blockade therapy in HNSCC and lead us to hypothesize that K17 is a strong predictive biomarker for HNSCC patients whose tumors are resistant to immune checkpoint blockade therapy.

### Results

### Human Head and Neck Cancers With High Levels of K17 Expression Have Shorter Survival Regardless of CD8+ Cell Level

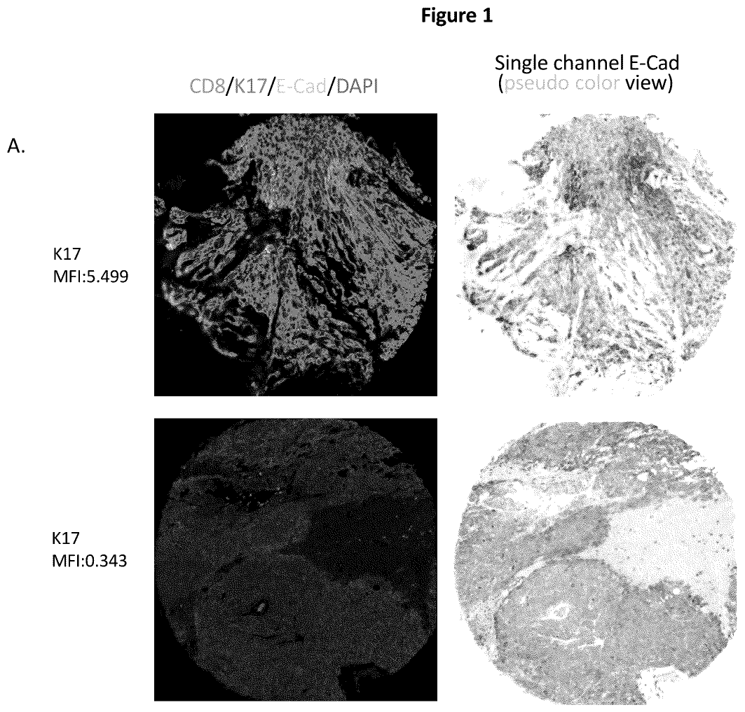

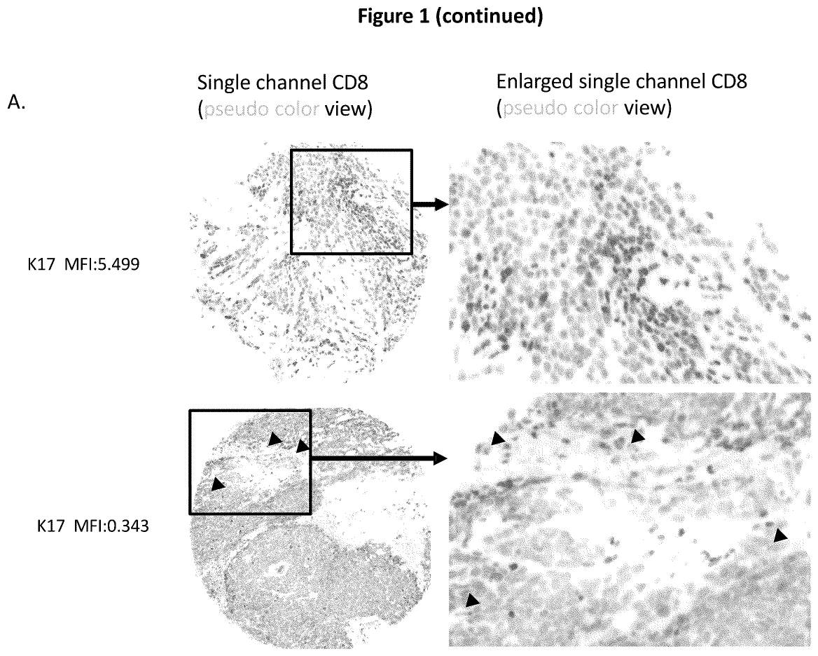

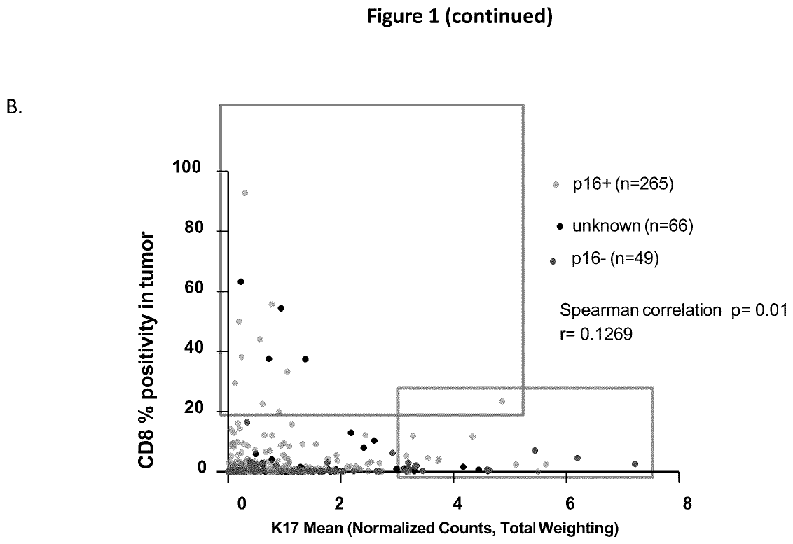

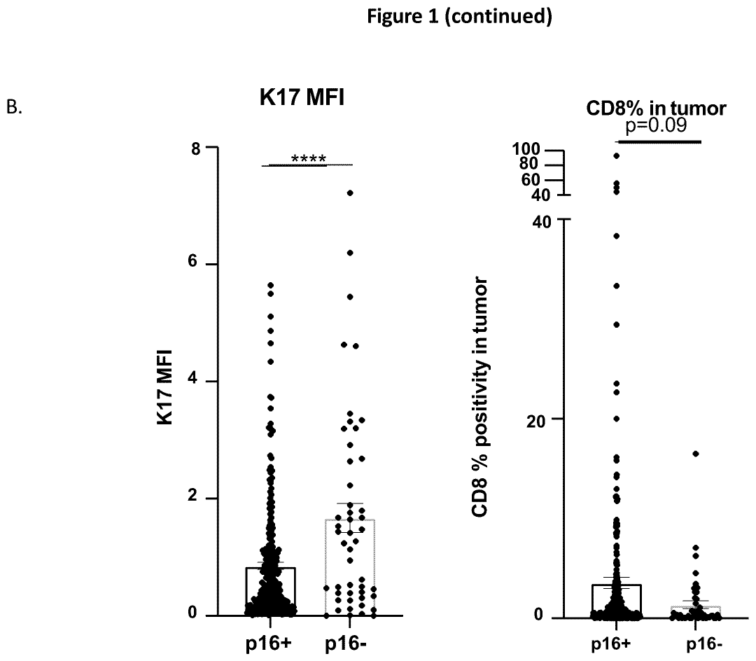

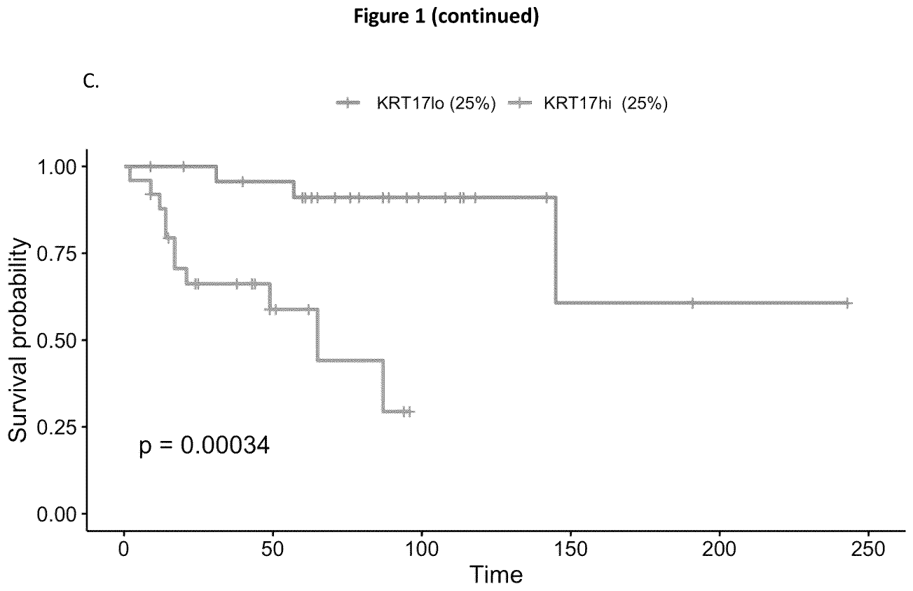

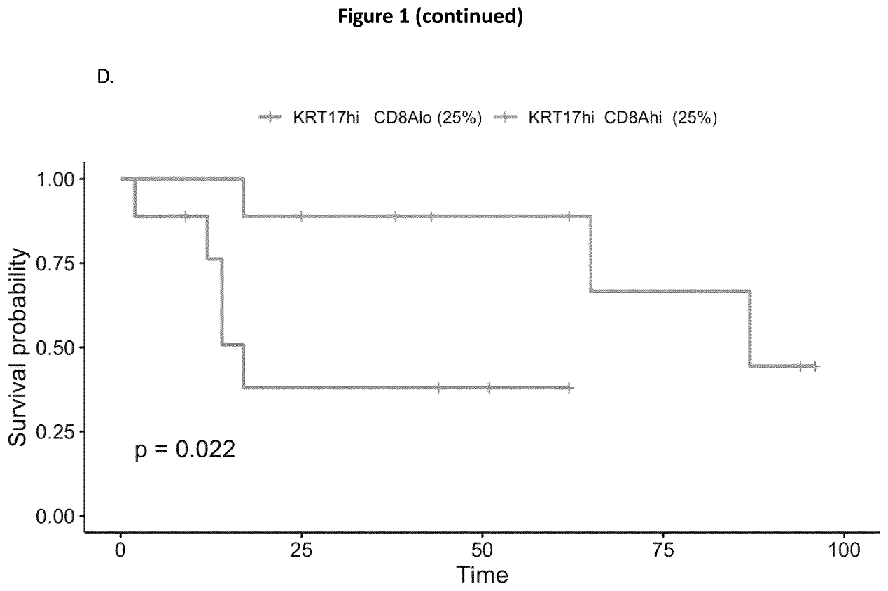

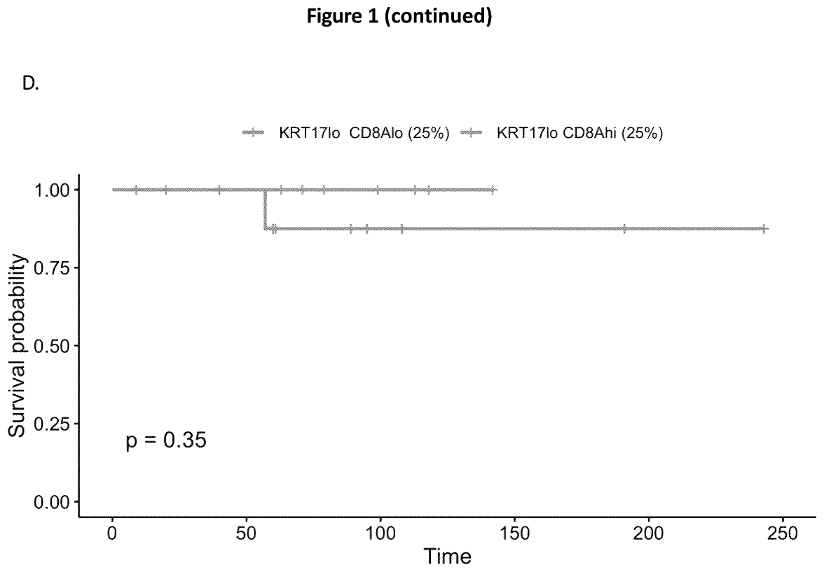

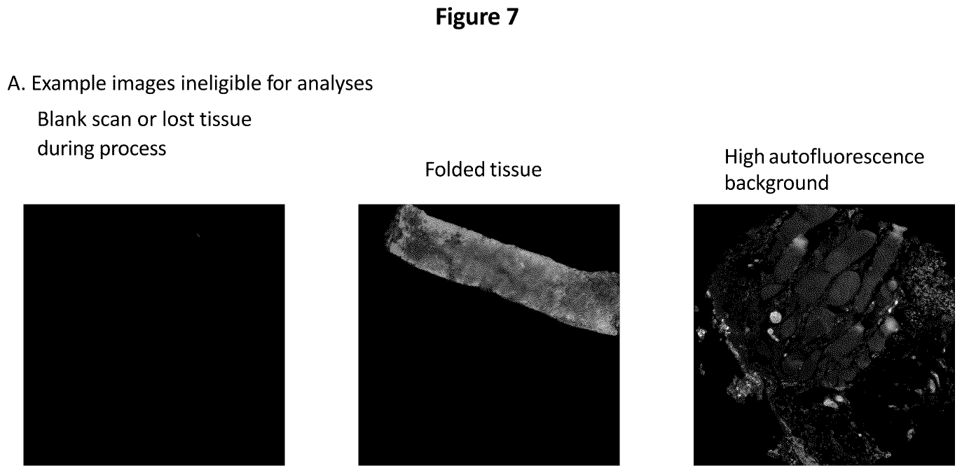

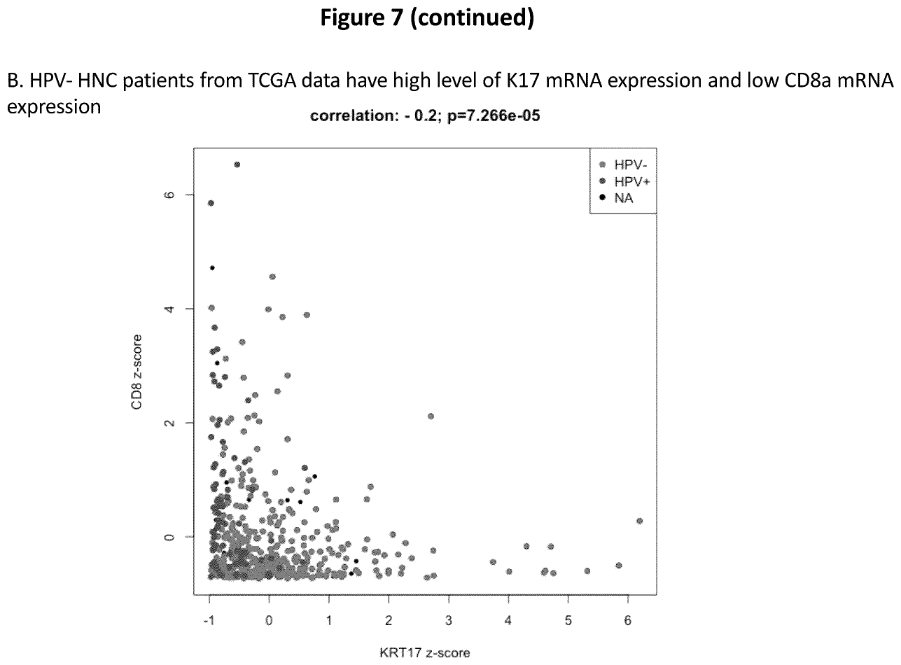

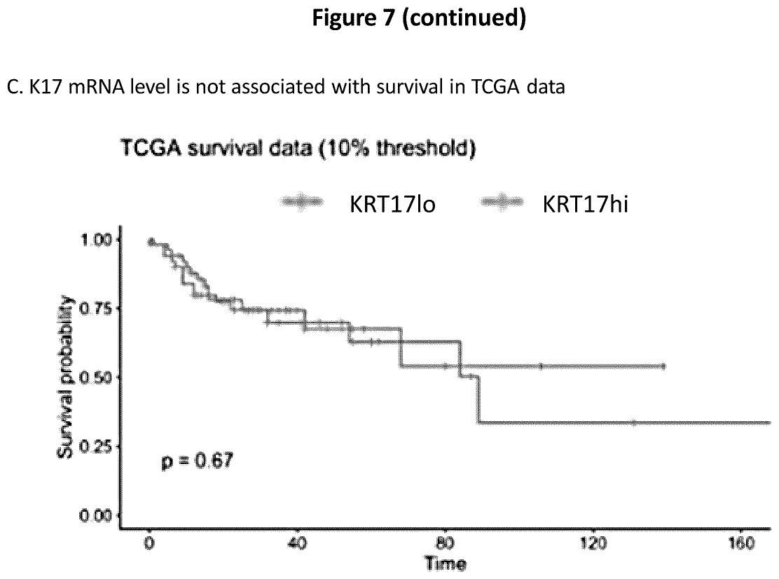

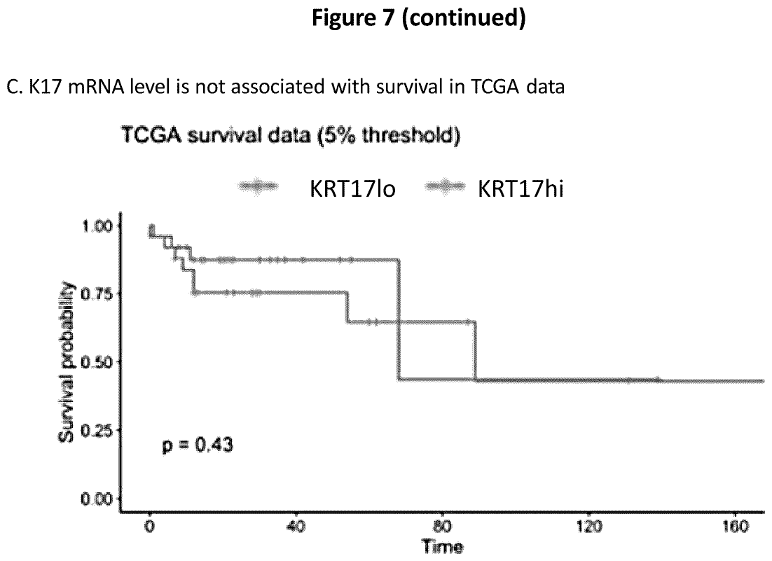

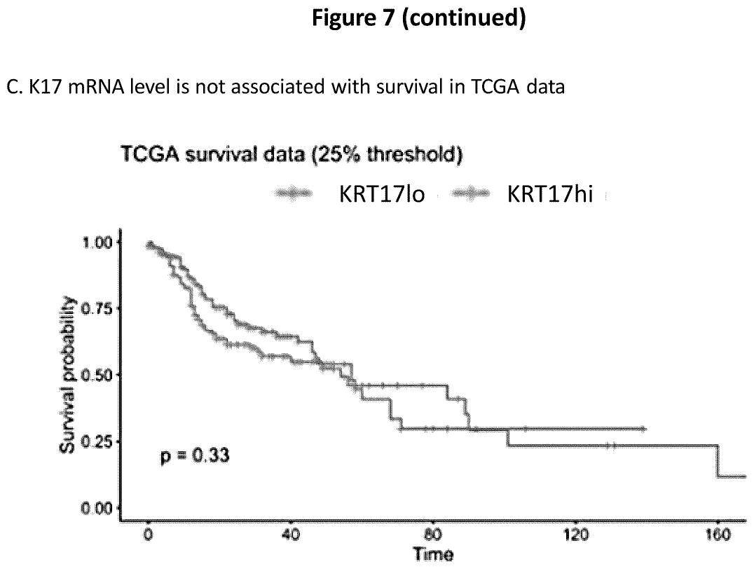

To test whether K17 is overexpressed in human head and neck cancers at the protein level, and whether high K17-expressing cancers have low abundance of infiltrating CD8+ T cells, we performed immunofluorescence staining on a tissue microarray (TMA, Table 1) containing both HPV+ and HPV− head and neck cancer patient specimens with K17, CD8 and E-cadherin specific antibodies. K17 mean fluorescence intensity (MFI) and CD8+ percent positivity were automatically calculated within or in close proximity to the E-cadherin+ regions (FIG. 1A). TMA cores lost or damaged (e.g. folded) during processing, or cores with high autofluorescence (FIG. 7A) were excluded from analyses. We found that, samples that were high in K17 expression (z score>1.64) all had less than 20% of intratumoral and peritumoral CD8+ T cell (blue box in FIG. 1B), and, conversely, samples that had high level of CD8+ T cell infiltration (z score>1.64) all had K17 MFI lower than 1.3 (red box in FIG. 1B), demonstrating that K17 high expressing tissues and CD8+ high infiltrating tissues are mutually exclusive (FIG. 1B, left). Interestingly, when we subclassified the tissue cores by their p16 status to distinguish HPV+ (p16+) from HPV− (p16−) cancers, we found HPV-samples had higher K17 expression and lower CD8 level compared to HPV+ samples (FIG. 1B middle and right). When we correlated survival time with K17 expression (average K17 expression from multiple primary cancers from the same patient), the highest 25% K17-expressing patients had significantly shorter survival than the lowest 25% K17-expressing patients (FIG. 1C). Among the high K17-expressing patients, their survival were further stratified by their CD8+ level (FIG. 1D left). The low K17-expressing patients had the best overall survival among all subgroups, regardless of intratumoral and peritumoral CD8+ level (FIG. 1D). Our analysis of K17 and CD8a RNA expression in the TCGA head and neck cancer patient RNA-seq data showed that, as seen with the TMA, tumors expressing high levels of K17 are mutually exclusive from tumors expressing high levels of CD8 (FIG. 7B). Also, as observed with the TMA data, the HPV-cancers had higher levels of expression for K17 than HPV+ cancers (FIG. 7B). However, K17 RNA level did not correlate with survival (FIG. 7C). This suggests to us that measurement of K17 at the protein level and within the tumor-specific region may be necessary for it to be used as a prognostic marker. These human patient data reveal that K17 is highly expressed in a subset of both HPV+ and HPV− human head and neck cancers, and, when scored at the protein level, is associated with poor survival in these patients. Because patients with HPV− HNSCC have higher K17 expression and lower CD8 level, we focused our studies on this cancer type.

TABLE 1
HNC tissue microarray patient characteristics

Patients (N = 105)

| | | |
|---|---|---|
| Characteristic | number of patients | percentage |

Age, mean (years) 59
Sex

| | | |
|---|---|---|
| Female | 17 | 16% |
| Male | 88 | 84% |

Race

| | | |
|---|---|---|
| Black | 1 | 1% |
| Hispanic | 1 | 1% |
| Unknown | 4 | 4% |
| White | 99 | 94% |

Primary tumor location

| | | |
|---|---|---|
| Tonsil | 102 | 97% |
| Soft Palate | 1 | 1% |
| Base of Tongue | 2 | 2% |

Disease stage

| | | |
|---|---|---|
| I | 1 | 1% |
| II | 9 | 9% |
| III | 25 | 24% |
| IV | 70 | 67% |

p16 status

| | | |
|---|---|---|
| p16− | 13 | 12% |
| p16+ | 73 | 70% |
| p16 Not known | 19 | 18% |

Initial treatment of primary disease

| | | |
|---|---|---|
| Chemoradiation | 24 | 23% |
| Radiation | 8 | 8% |
| Surgery | 73 | 70% |

Post-op treatment of primary disease

| | | |
|---|---|---|
| Chemoradiation | 14 | 13% |
| Radiaiton | 30 | 29% |
| Additional surgery | 4 | 4% |
| None | 57 | 54% |

### K17 Supports MOC2 In Vivo Growth in Immunocompetent Mice but is Not Required for Growth in Immunodeficient Mice

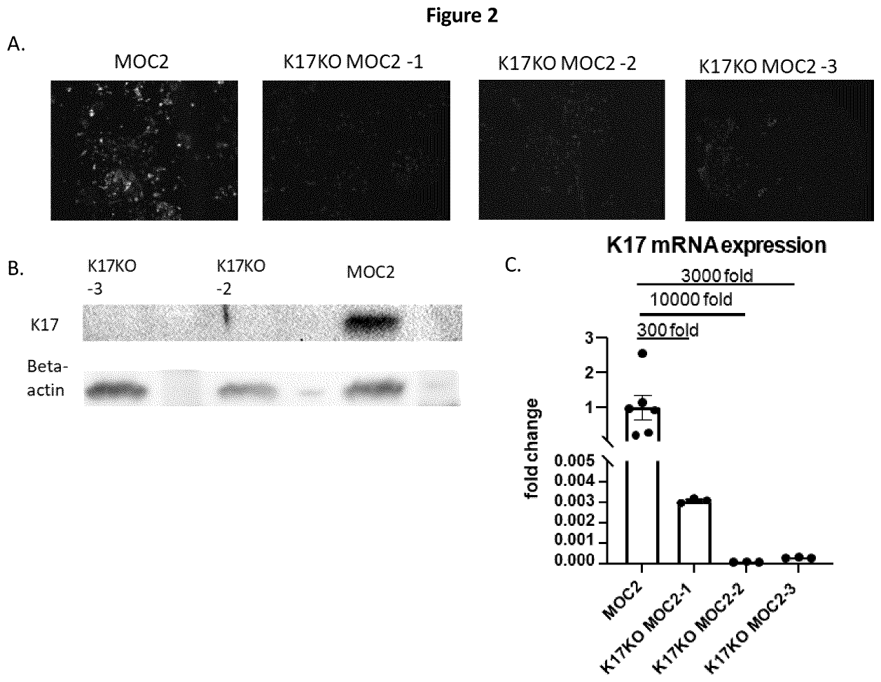

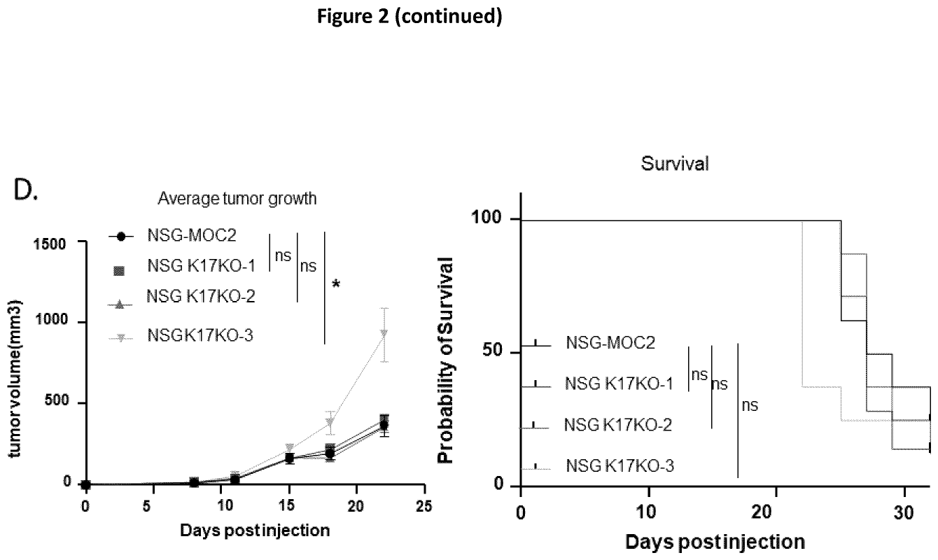

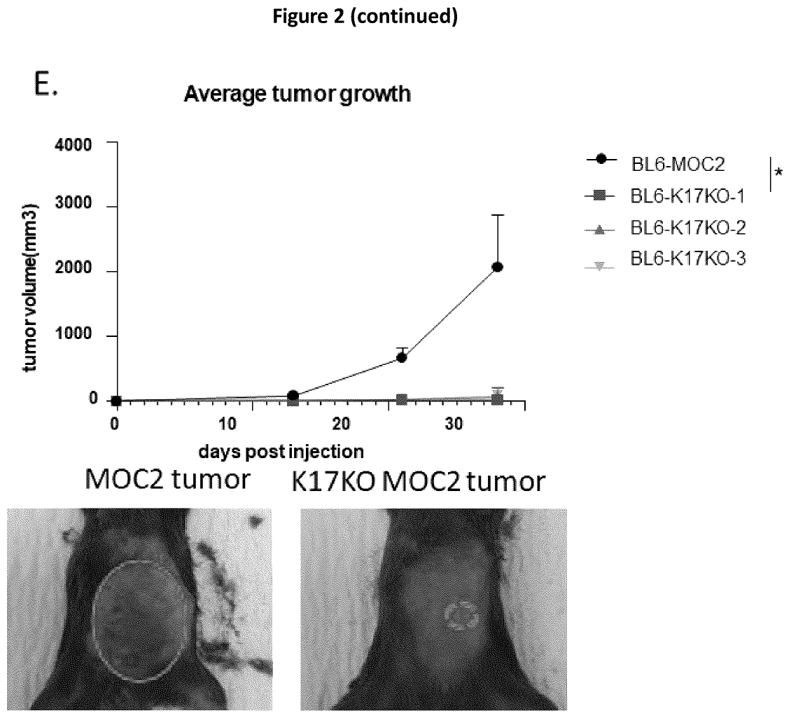

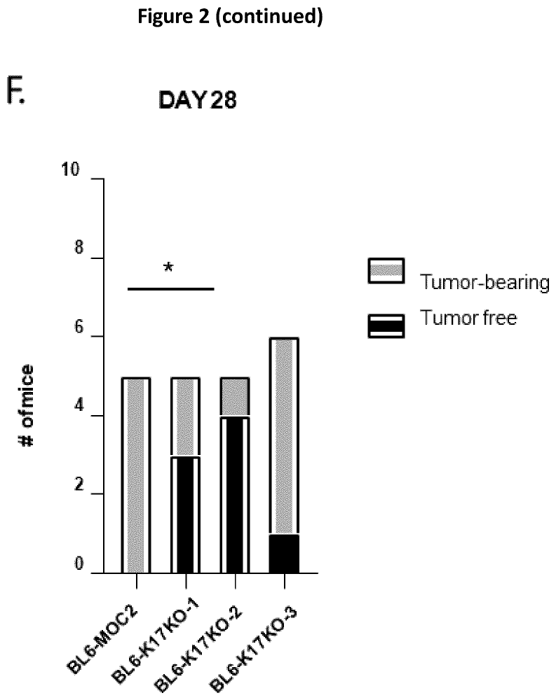

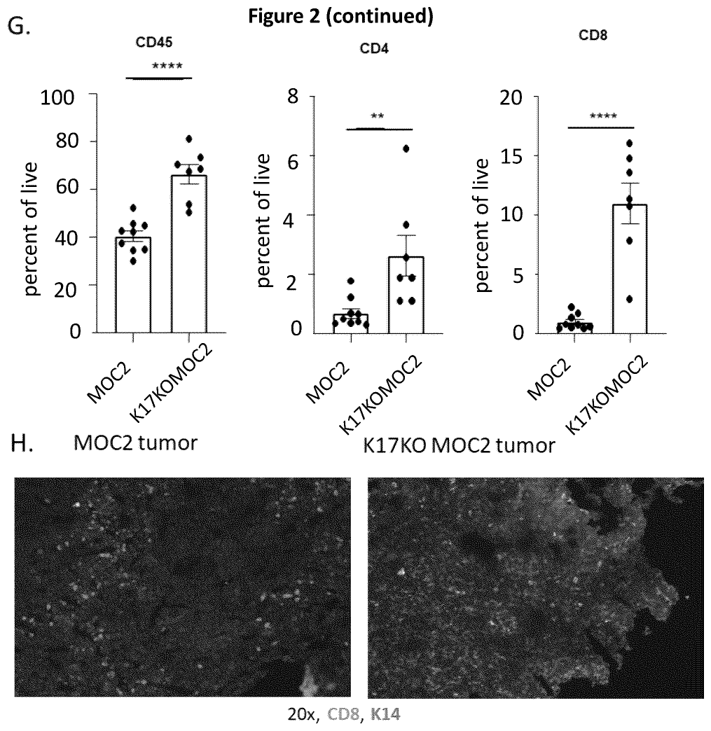

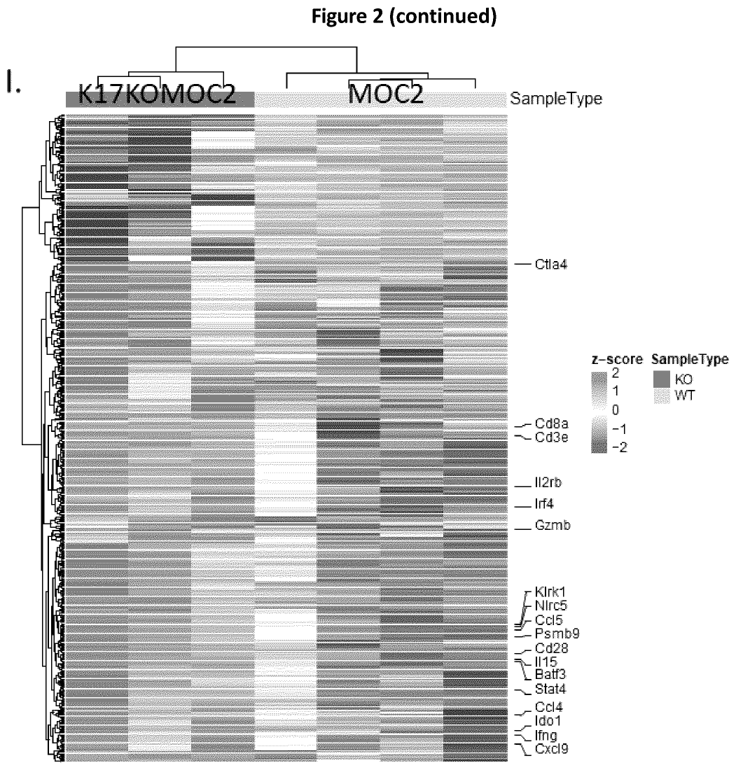

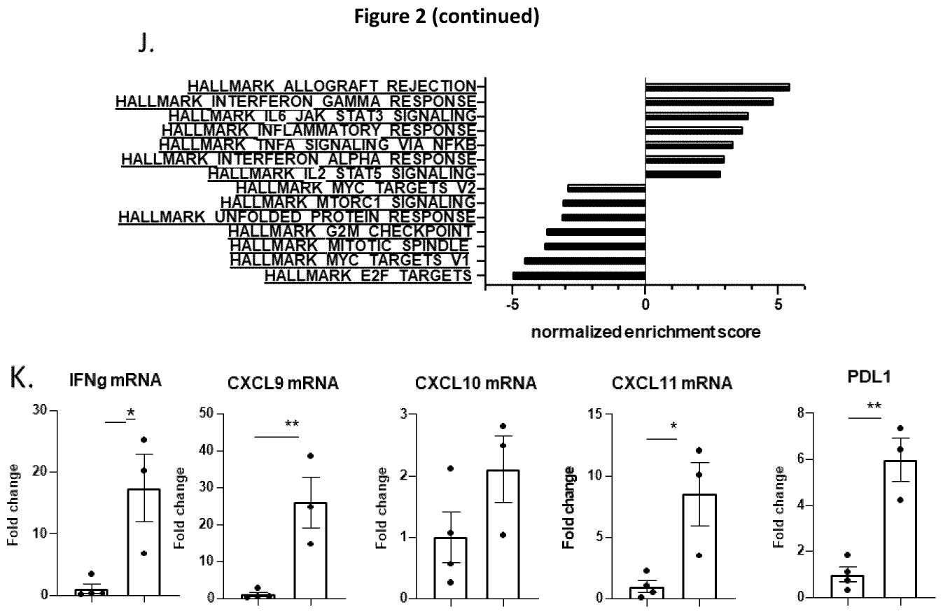

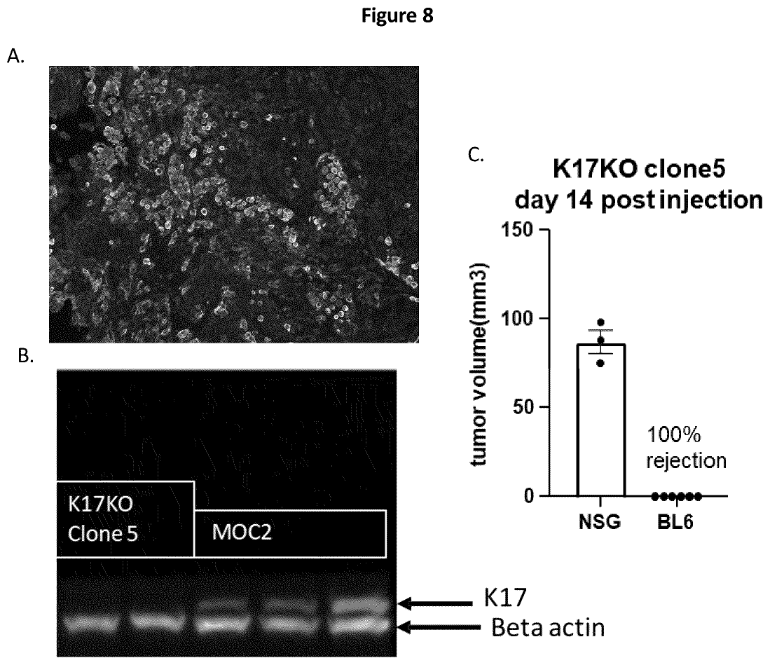

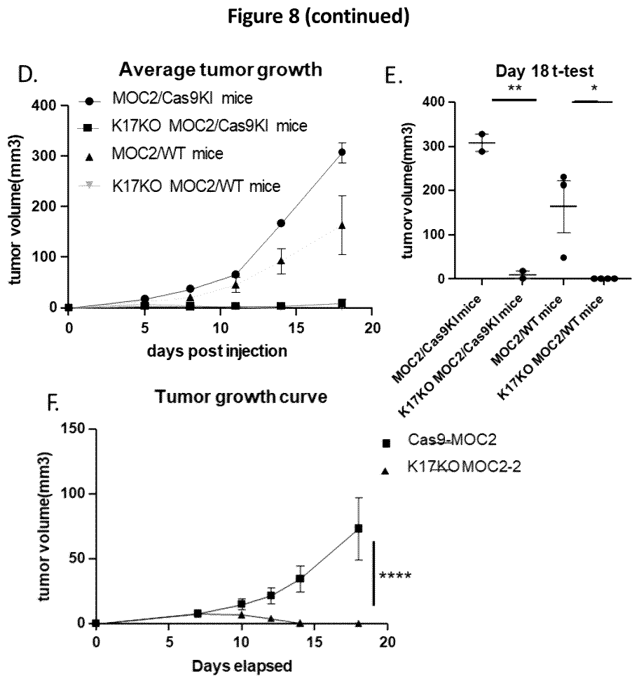

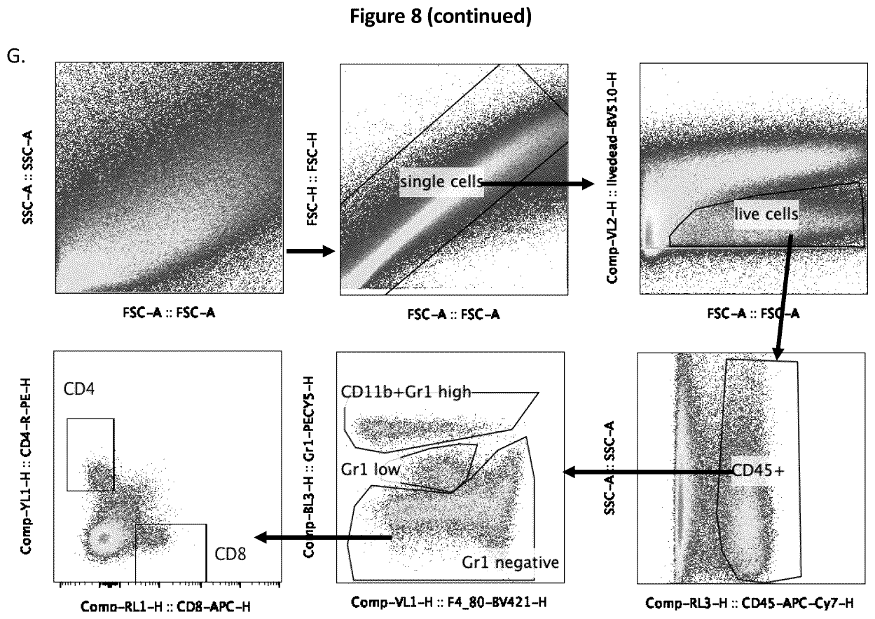

MOC2 represents a valuable syngeneic (C57BL/6) mouse head and neck cancer model for our studies because it represents an immunologically cold tumor phenotype and it expresses K17 (FIGS. 2A and 2B). We designed CRISPR/Cas9 guide sequences targeting Exon 1, Exon 4 and Exon 5 of the mouse K17 gene to generate three independent K17 knockout (K17KO) MOC2 cell bulk populations that were expanded from the parental cells that had been transduced with a retroviral vector that expressed Cas9 together with the appropriate guide RNA. These bulk populations were maintained under drug selection during their culture in vitro and were found to have undetectable K17 protein expression (FIGS. 2A and 2B) and very low levels of K17 mRNA (FIG. 2C). When subcutaneously injected into immune-deficient NOD scid gamma (NSG) mice, one of the three K17KO MOC2 cell lines grew slightly faster than the parental wildtype MOC2 cells, with a 100% growth incidence, while the other two K17KO MOC2 cell lines formed tumors that grew nearly identically to tumors arising in mice injected with parental MOC2 cells (FIG. 2D). Mice bearing K17KO MOC2 lines had similar survival compared to those bearing MOC2 tumors (FIG. 2D), suggesting that knocking out K17 did not reduce the tumorigenicity of MOC2 cells. However, when injected into syngeneic, immunocompetent, C57BL/6 mice, all three K17KO MOC2 cell lines had significantly delayed growth (FIG. 2E), with a 50% complete rejection rate on average (FIG. 2F). The K17KO-3 cells had the lowest rejection rate among the three K17KO lines generated. We believe this might be because bulk populations retained cells in which K17 was not successfully knocked out and these K17+ cells then expanded in vivo, during which time the cells could not be maintained under drug selection, as evidenced by the presence of K17+ tumor cells in the resulting tumors from bulk populations (green cells in FIG. 8A). For this reason, we went back and generated single cell clones of K17KO-3 that were confirmed to be K17-negative by western blot analysis (FIG. 8B). When these cells were injected into syngeneic mice, 100% of the tumors were rejected (FIG. 8C), consistent with our hypothesis that leaky expression of K17 in the bulk populations likely contributed to variable levels of rejection. The observed, delayed growth of K17KO MOC2 tumors was unlikely due to Cas9 expression in K17KO MOC2 cells, because K17KO MOC2 tumors had comparable delayed growth and rejection rates in Cas9 Knock-in (Cas9KI) mice that do not recognize Cas9 as a foreign antigen (FIGS. 8D and 2E), and because K17KO MOC2 tumors grew significantly slower than MOC2 cells expressing Cas9 only (FIG. 8F). Flow cytometry of remaining K17KO MOC2 tumors and parental MOC2 tumors revealed significantly higher numbers of CD45+, CD4+ and CD8+ immune cell infiltrates in K17KO MOC2 tumors (FIG. 2G, FIG. 8G). Infiltration of CD8+ T cells was confirmed by immunofluorescence staining (FIG. 2H). These data are consistent with the hypothesis that K17 inhibits immune-mediated spontaneous rejection of head and neck cancer cells in immunocompetent mice and that ablation of K17 expression creates an immunologically hot tumor microenvironment.

Next, we performed RNA sequencing (RNA-Seq) of bulk RNA extracted from the persisting, slow growing K17KO MOC2 tumors as well as parental MOC2 tumors from C57BL/6 mice. We identified 115 downregulated genes and 388 upregulated genes (adjusted p<0.05, log 2FC<−2 or >2) in K17KO MOC2 tumors compared to MOC2 tumors. Among the upregulated genes, we observed active cellular immune response genes, including CD8a, CD28, Grzmb, Batf3, and CXCL9 (FIG. 2I). Gene Set Enrichment Analysis (GSEA) (31) defined seven upregulated pathways in K17KO MOC2 tumors that were all related to immune response, including IFN alpha and IFN gamma responses (FIG. 2J). We confirmed upregulation of IFNg-related gene expression by qRT-PCR analyses of the bulk tumor RNA, including IFNg, CXCL9 and PD-L1 (FIG. 2K). On the other hand, the top enriched pathways in downregulated genes were cell cycle and cell proliferation-related, including E2F targets, MYC targets and mitotic spindle-associated genes (FIG. 2J). These findings were consistent with the delayed growth kinetics of K17KO MOC2 tumors in the C57BL/6 mice (FIG. 2E). Overall, the RNA-Seq data provide strong evidence for a major switch in the immune signature of MOC2 vs K17KO MOC2 tumors, with K17KO tumors possessing a more activated T cell and immunogenic tumor microenvironment that correlates with a strong reduction in cell growth parameters.

### T Cells are Responsible for Rejection of K17KO MOC2 Cells in C57BL/6 Mice

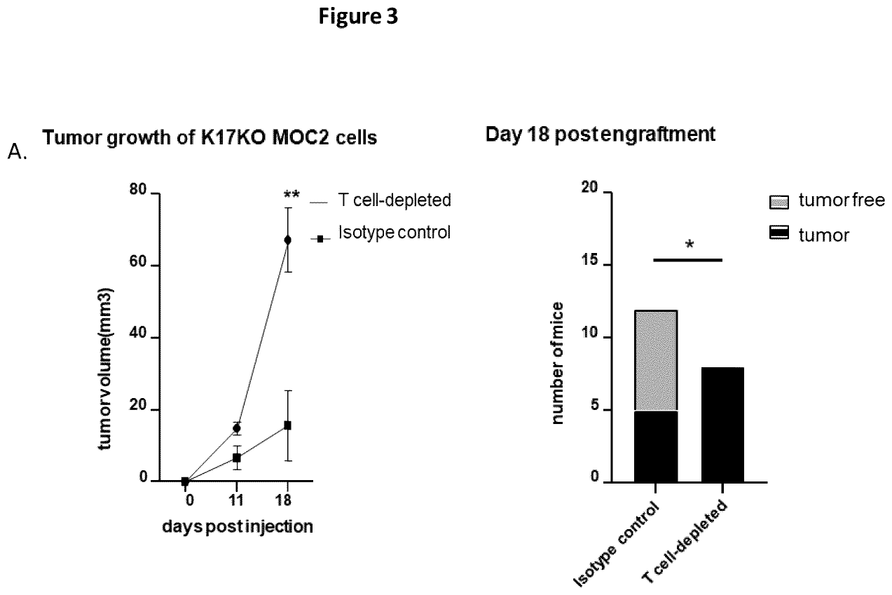

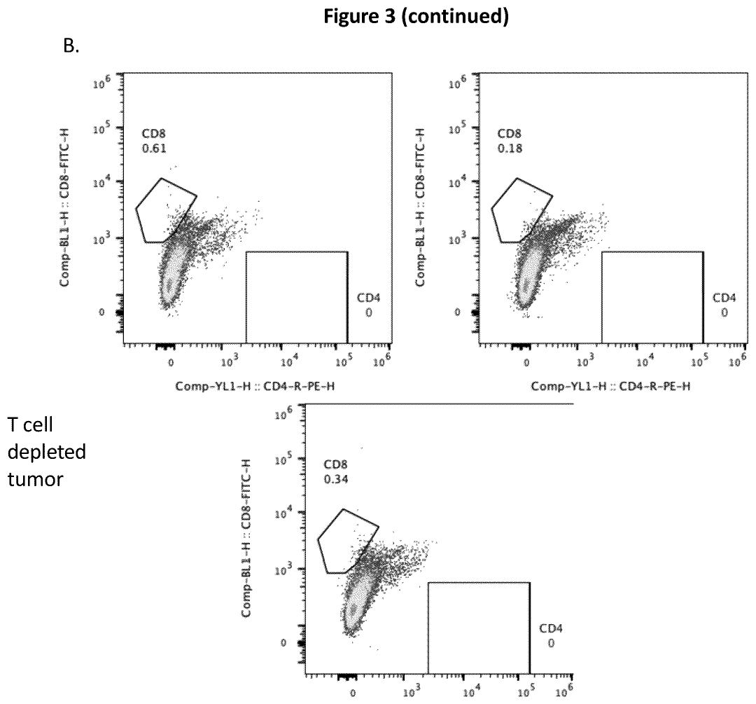

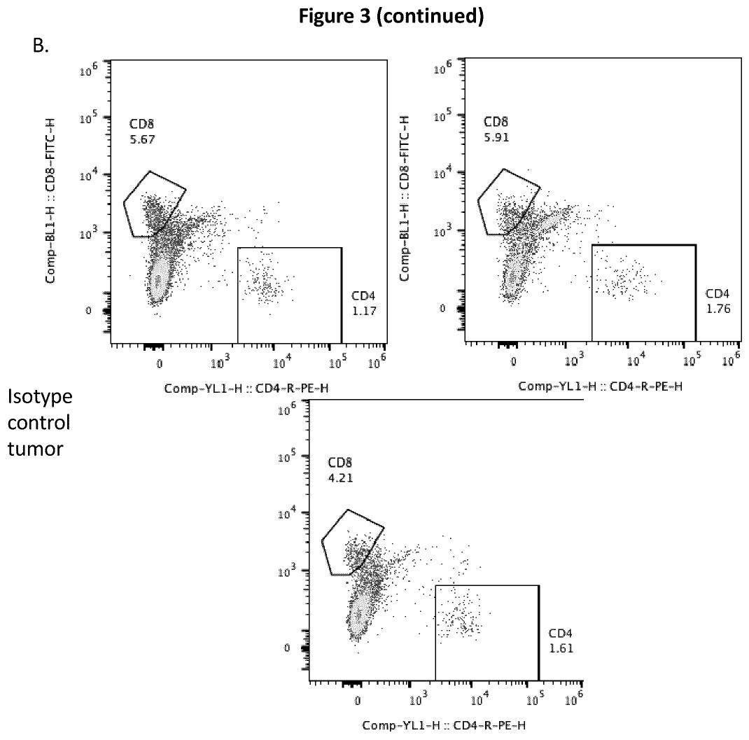

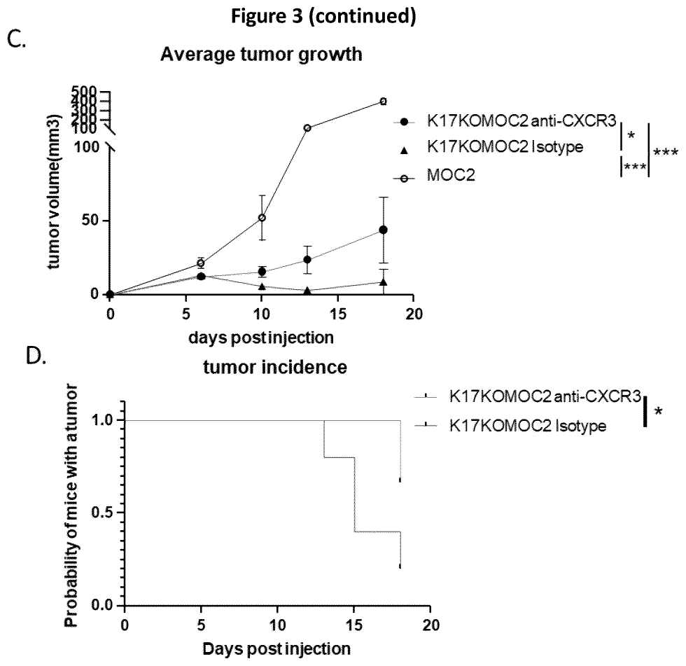

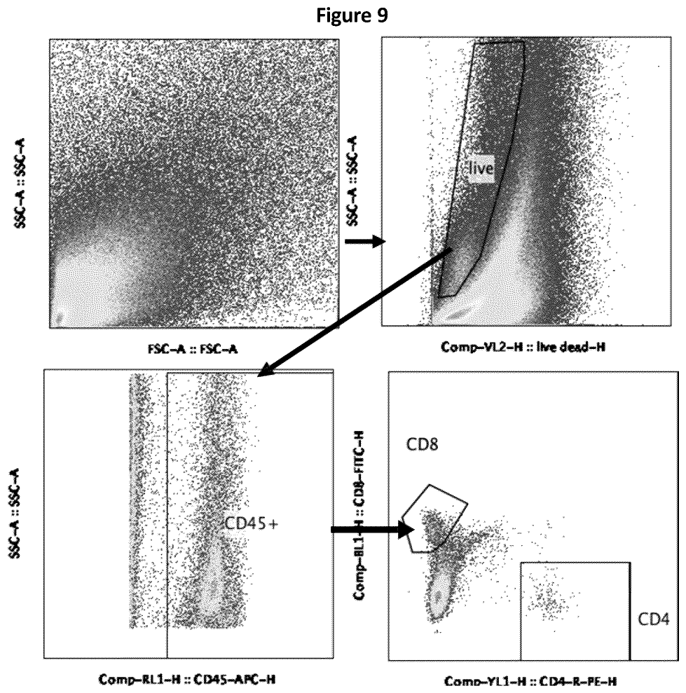

Based on our data described above, we hypothesized that infiltrating T cells were responsible for the rejection and slow growth of K17KO MOC2 tumors in syngeneic, immunocompetent mice. We therefore depleted CD4+ and CD8+ T cells from C57BL/6 mice beginning at three days post injection of K17KO MOC2 cells and continued depleting these cells through the time course of the study. We found that none of the K17KO MOC2 tumors were rejected in mice depleted for T cells, and that these tumors grew significantly larger than the K17KO MOC2 tumors growing in mice treated with isotype control antibodies (FIG. 3A). The absence of T cells in the K17KO MOC2 tumors growing in T cell-depleted mice was confirmed by flow cytometry (FIG. 3B, FIG. 9). These data confirm the hypothesis that the spontaneous rejection of K17KO MOC2 tumors in immunocompetent C57BL/6 mice is T-cell dependent.

Because we observed increased levels of CXCL9 and CXCL11 RNA expression, chemokines that attract activated CXCR3-expressing T and NK cells, in K17KO MOC2 tumors (FIG. 2K), we tested whether CXCR3 was important in the rejection of K17KO MOC2 tumors in vivo. Anti-CXCR3 antibody was delivered to mice by i.p. to block CXCR3's interactions with its ligands one day before injection of K17KO MOC2 cells. In isotype control treated mice bearing K17KO MOC2 tumors, the tumors grew significantly slower than K17KO MOC2 tumors in CXCR3-blocked mice (FIG. 3C). Blocking CXCR3 also significantly delayed the rejection of K17KO MOC2 tumors (FIG. 3D). However, blocking CXCR3 did not completely abrogate the anti-tumor immune effect against K17KO MOC2 tumors, as they still grew more slowly than the parental MOC2 tumors (FIG. 3C); even with CXCR3 blockade two K17KO MOC2 tumors were completed rejected (FIG. 3D), suggesting there are other factors that contribute to rejection of K17KO MOC2 besides CXCL9/CXCL11 and CXCR3 interactions.

### K17KO MOC2 Cell-Immunized Mice Generate Partially Protective Memory Responses Against Parental MOC2 Cells

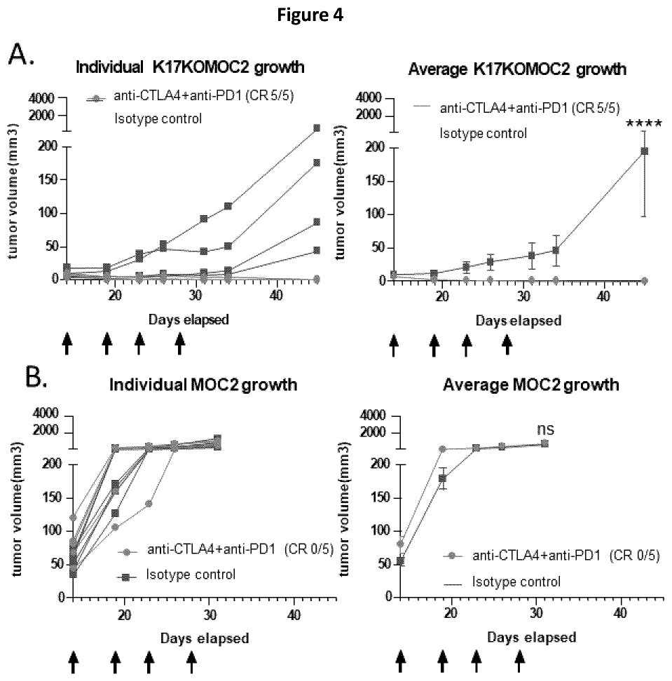

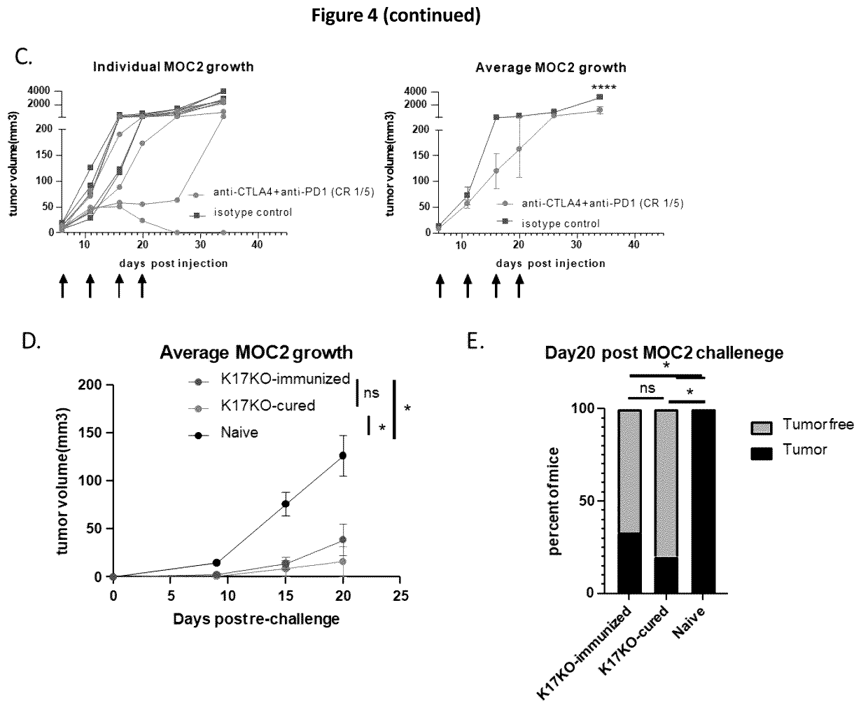

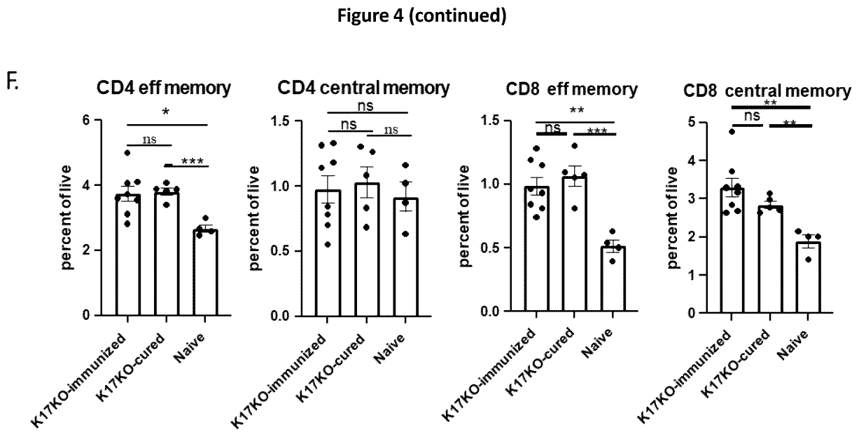

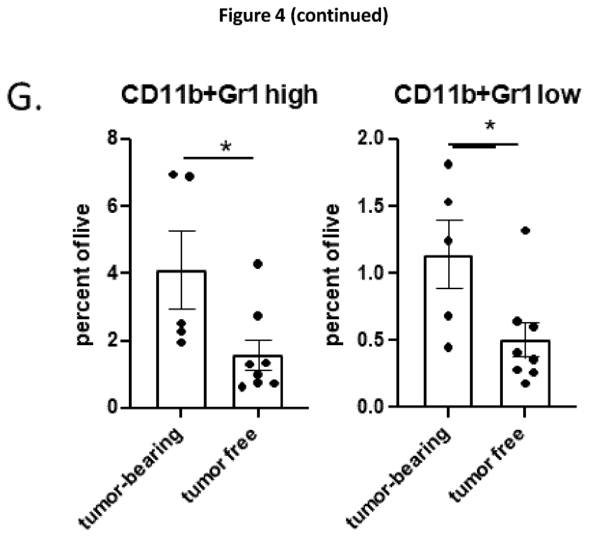

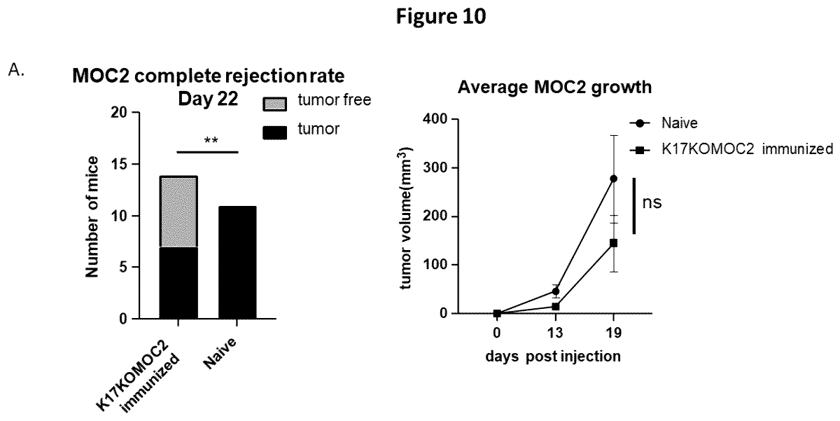

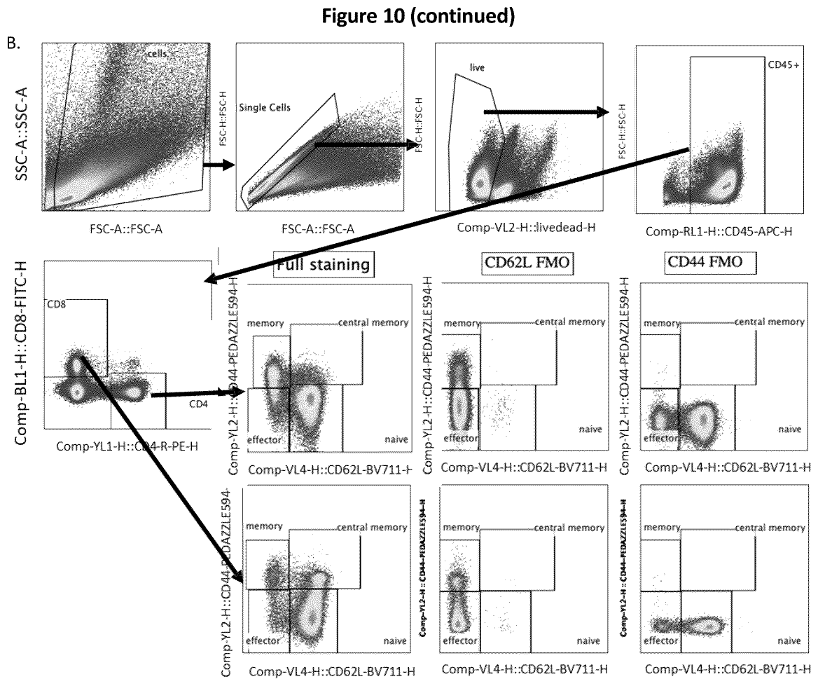

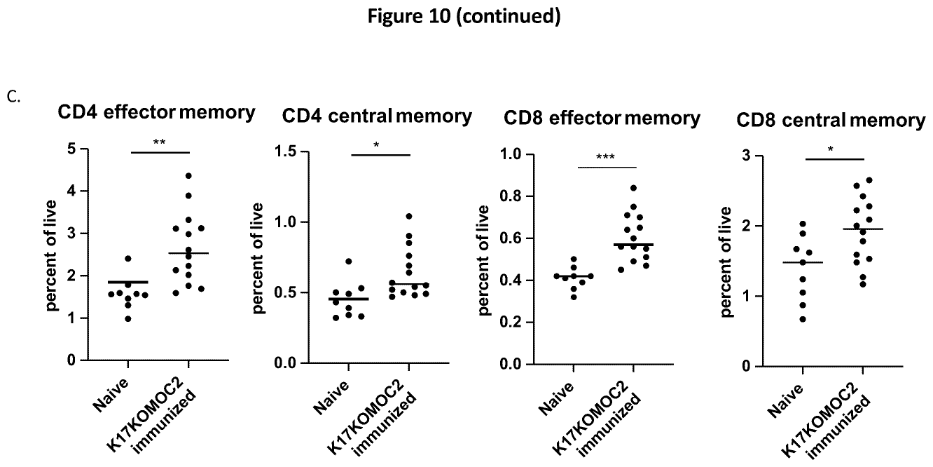

To test if the immune response elicited by K17KO MOC2 cells are not solely caused by potential neoantigens resulting from CRISPER/Cas9 editing, we re-challenged those C57BL/6 mice that were able to completely clear K17KO MOC2 cells (‘K17KOMOC2-immuned mice’) with parental MOC2 cells. We found that half of the immunized mice were able to completely reject parental MOC2 tumor growth, while none of naïve mice rejected parental MOC2 tumors (FIG. 10A, left). We also observed a slightly but not significantly delayed growth of MOC2 tumors in the K17KOMOC2-immunized mice (FIG. 10A, right). When we analyzed splenocytes from K17KOMOC2-immunized mice and naïve mice upon MOC2 tumor challenge, we found an increased number of CD4+ and CD8+ T cells that had memory markers (CD62L−CD44 high and CD62L+CD44 high) (FIGS. 10B and 4C). These data indicate that the immune response elicited by K17KO MOC2 cells are able to exert partial protection against parental MOC2 tumor challenge, suggesting that K17KO MOC2-induced immune responses are able to recognize antigens present in parental MOC2 cells.

### K17KO MOC2 Tumors Have Enhanced Response to Immune Checkpoint Blockade Treatment

Next, we tested whether the immunologically hot K17KO tumors had increased response to immune checkpoint blockade treatment. At day 14 post injection, about half of K17KO MOC2 tumors were completely rejected. K17KO MOC2 tumors that were larger than 2 mm×2 mm on day 14 were defined as tumors that survived natural immune surveillance because they continued to grow over time. C57BL/6 mice carrying these >2 mm×2 mm, persisting K17KO MOC2 tumors were treated with anti-PD1+anti-CTLA4 antibodies or isotype controls starting at 14 days post-injection of the tumor cells. Isotype control treated K17KO MOC2 tumors either continued to grow or maintained the same size for three weeks and started to grow in size after day 35 post-injection (FIG. 4A). In contrast, all five anti-PD1+anti-CTLA4-treated K17KO MOC2 tumors completely regressed by day 45 post-injection (FIG. 4A). On the other hand, the parental MOC2 tumors that were treated at the same time (day 14 post injection) did not respond to anti-PD1+anti-CTLA4 treatment (FIG. 4B). Due to the significant difference in size of K17KO MOC2 tumors versus MOC2 tumors at the time we initiated immune checkpoint blockade treatment, we also treated a cohort of mice bearing parental MOC2 tumors that had a size similar to that of the K17KO MOC2 tumors, starting at day 6 post injection. One out of five MOC2 tumors treated from day 6 post injection had a complete response, one MOC2 tumor had a delayed growth, the other three MOC2 tumors continued to grow despite ICB treatment (FIG. 4C). Therefore, we conclude that, even when accounting for initial tumor size, the rejection of tumors in response to immune checkpoint blockade treatment is significantly higher for K17KO MOC2 tumors (CR: 5/5, FIG. 4A) compared to parental MOC2 tumors (CR: 1/5, FIG. 4C) (p<0.05, Fisher's exact test).

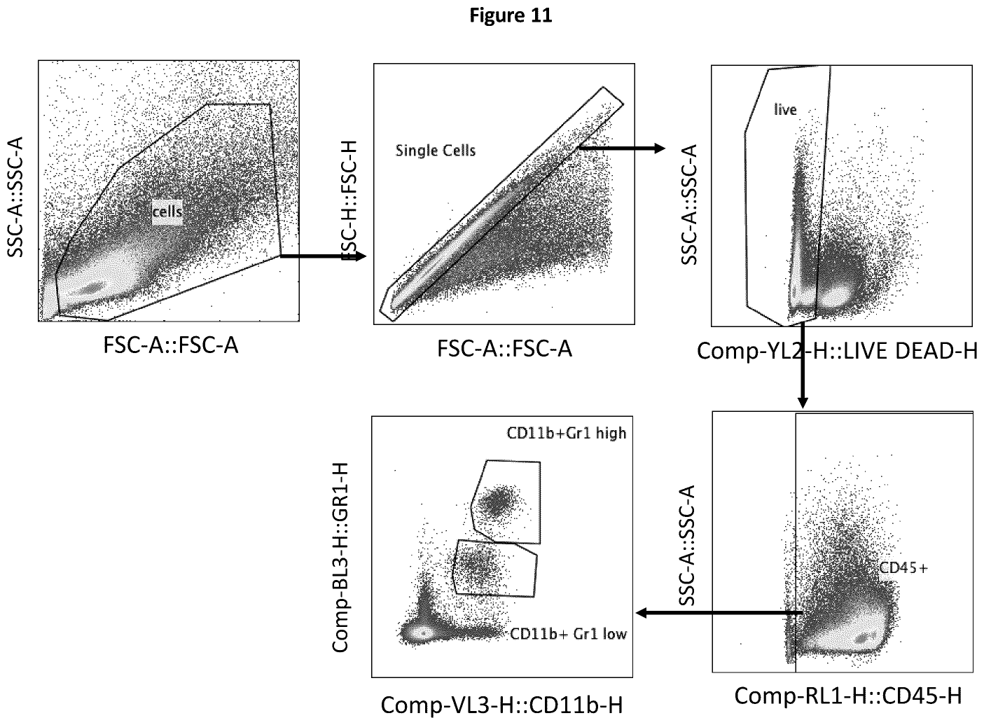

To test whether anti-PD1+anti-CTLA4-treated mice bearing K17KO MOC2 tumors that then regressed generated protective memory response to parental MOC2 cells, we rechallenged the five mice that completely cleared K17KO MOC2 upon immune checkpoint blockade treatment (‘K17KO-cured mice’), as well as twenty-four mice that spontaneously cleared K17KO MOC2 tumors (‘K17KO-immunized’), with parental MOC2 cells. MOC2 tumor growth was significantly delayed in both K17KO-immunized mice and K17KO-cured mice compared to naïve mice (FIG. 4D). Among these mice, 65% of the K17KO-immunized mice were able to completely reject MOC2 tumor growth, versus 80% of ICB-treated, K17KO-cured mice (FIG. 4E). CD62L and CD44 analyses of splenocytes from these mice indicated that higher numbers of CD4+ and CD8+ T cells with memory phenotypes were present in the immunized mice compared to naïve mice (FIG. 4F). Altogether, these data demonstrate that the formation of memory response against MOC2 tumors occurred in both K17KO-immunized mice and K17KO-cured mice, with no significant difference in protection against MOC2 challenge. Interestingly, the frequencies of CD11b+Gr1 high and CD11b+Gr1 low cells were higher in the spleens of mice that were unable to reject MOC2 tumors (tumor-bearing), compared to the spleen of mice that rejected MOC2 tumors (tumor free) (FIG. 4G, FIG. 11), indicating that myeloid-derived suppressor cells might play a role in supporting MOC2 tumor outgrowth in K17KO-immunized mice.

### High Level of K17 Expression in HNSCC Patients is Associated With Poor Response to Pembrolizumab

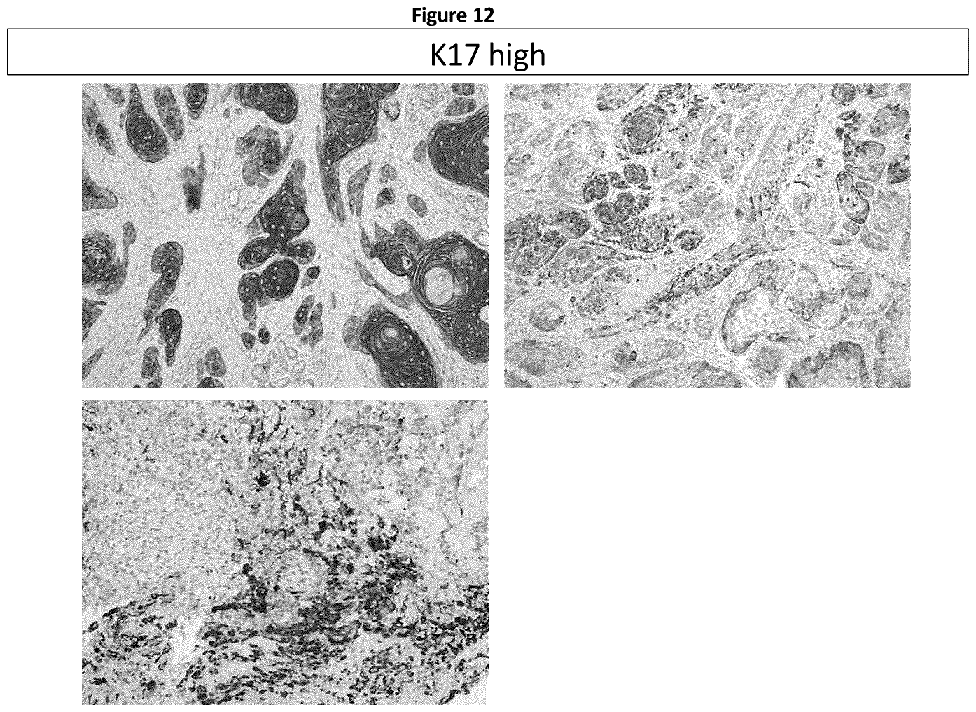

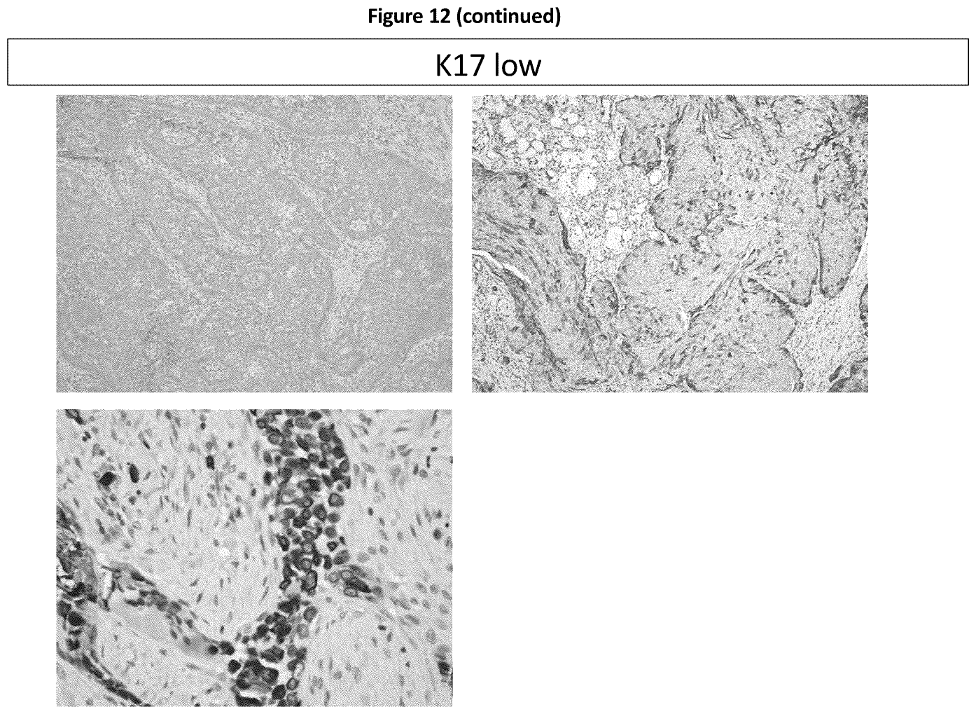

To investigate the human relevance of our findings that K17 status influences response to ICB in mice, we evaluated K17 expression by immunohistochemistry in a cohort of 26 HNSCC patients receiving Pembrolizumab (Table 2) and looked for whether there was a correlation between their level of K17 expression and their clinical response. Based on high expression cut-off of >5% strong cytoplasmic staining of tumor cells, 18 (69.2%) patients had K17 high expressing tumors and 8 (30.8%) had K17 low expressing tumors (FIG. 5A and FIG. 12). Disease control rate was significantly associated with K17 expression status (p<0.001, FIG. 5B). In the K17 high group, all patients had progressive disease. In the K17 low group, 6 patients (75%) had disease controlled, while 2 (25%) patients had progressive disease. In addition, K17 low patients had significantly longer overall survival (p=0.02, FIG. 5C) and progression-free survival (p=0.004, FIG. 5D). These clinical data support our hypothesis that high K17 expression in head and neck cancers confers resistance to ICB therapy.

TABLE 2
Characteristics of HNC patients who received Pembrolizumab

| | Patients (N = 26) | |
|---|---|---|
| Characteristic | number of patients | percentage |

Age, median (years) 60.5
Sex

| | | |
|---|---|---|
| Female | 4 | 15% |
| Male | 22 | 85% |

ECOG performance status

| | | |
|---|---|---|
| 0 | 5 | 19% |
| 1 | 16 | 62% |
| 2 | 4 | 15% |
| 3 | 1 | 4% |

Smoking status

| | | |
|---|---|---|
| Current or former | 21 | 81% |
| Never | 5 | 19% |

Disease status

| | | |
|---|---|---|
| Metastatic | 19 | 73% |
| Local recurrence | 7 | 27% |

Primary tumor location

| | | |
|---|---|---|
| Oral cavity | 14 | 54% |
| Oropharynx | 6 | 23% |
| Larynx | 1 | 4% |
| Paranasal sinus | 2 | 8% |
| Other | 3 | 12% |

Tumor grade

| | | |
|---|---|---|
| Well differentiated | 4 | 15% |
| Moderately differentiated | 12 | 46% |
| Poorly differentiated | 5 | 19% |
| Not graded | 5 | 19% |

HPV P16

| | | |
|---|---|---|
| Positive | 9 | 35% |
| Negative | 8 | 31% |
| Not performed/not found | 9 | 35% |

PD-L1 expression by IHC

| | | |
|---|---|---|
| High | 11 | 42% |
| Low | 3 | 12% |
| Not performed | 12 | 46% |

Checkpoint inhibitor regimen

| | | |
|---|---|---|
| Pembrolizumab | 24 | 92% |
| Pembrolizumab/5FU/carboplatin | 2 | 8% |

Number of doses, median (range) 3 (1-11)
Best Response

| | | |
|---|---|---|
| Disease control | 6 | 23% |
| Progressive disease | 20 | 77% |

Concomitant radiation

| | | |
|---|---|---|
| Yes | 2 | 8% |
| No | 24 | 92% |

### K17KO MOC2 Tumors Have Switched Tumor Immune Microenvironment Phenotypes

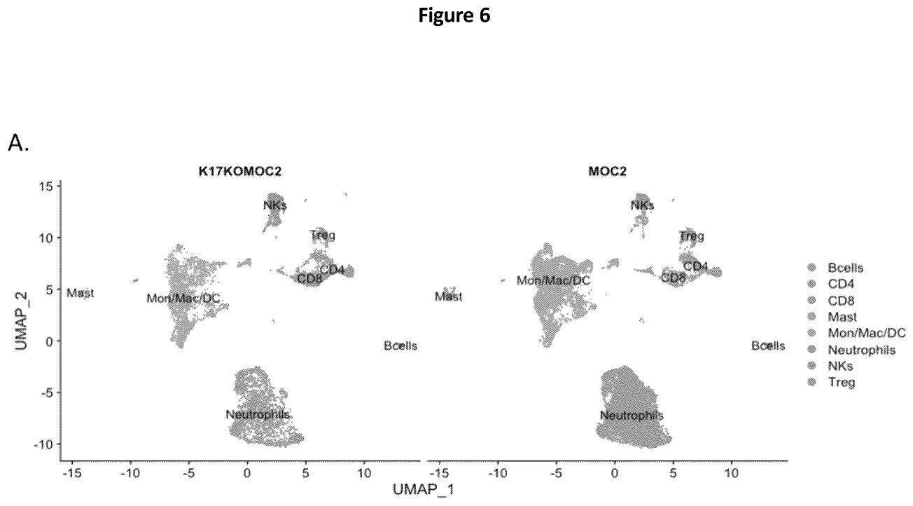

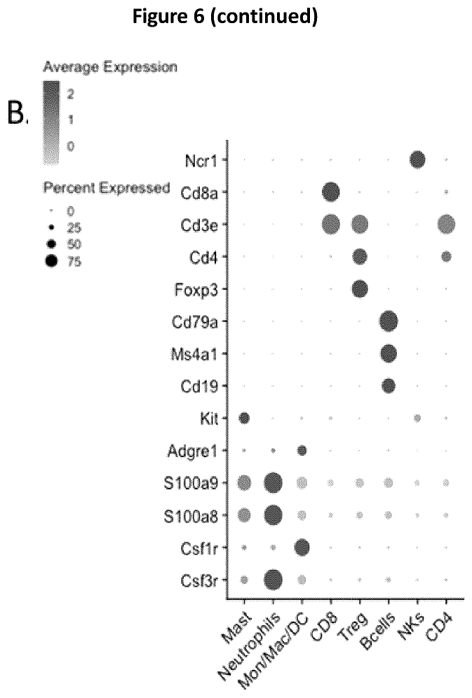

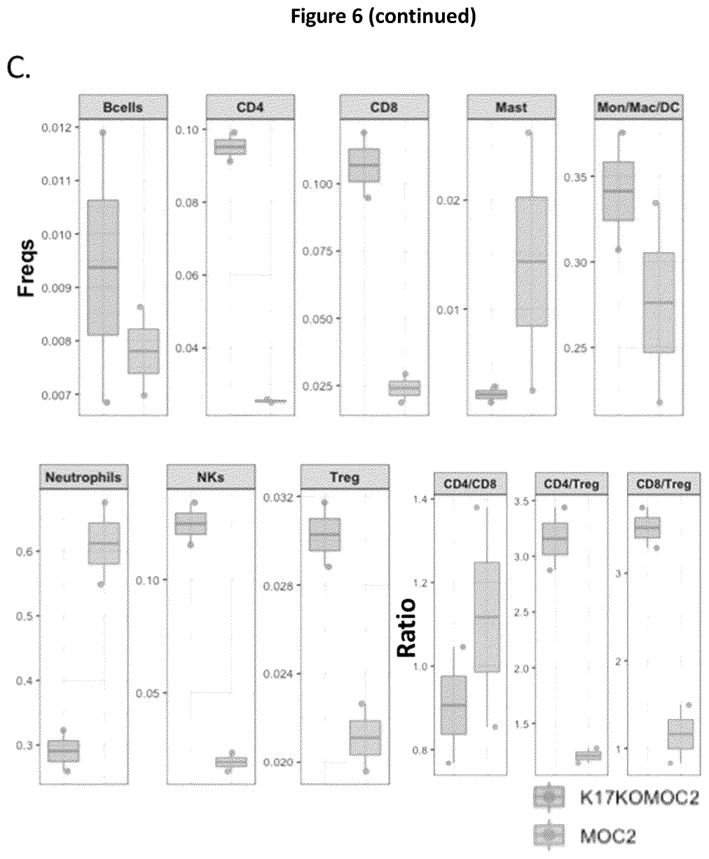

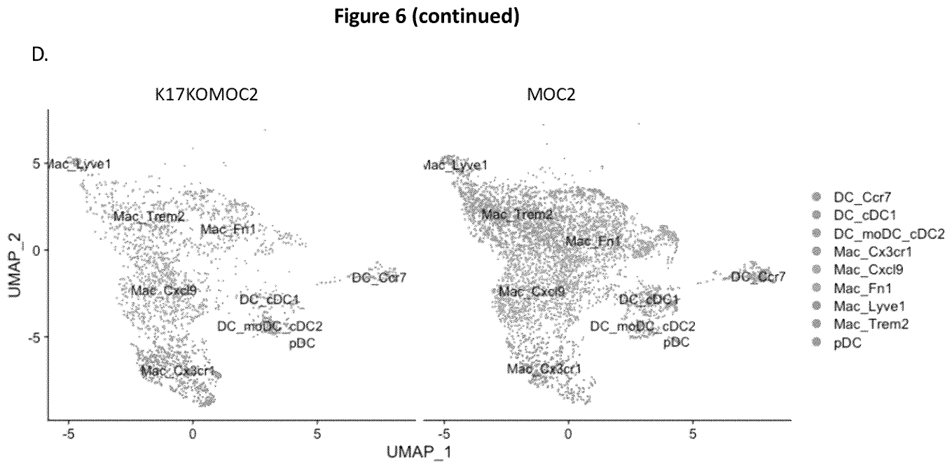

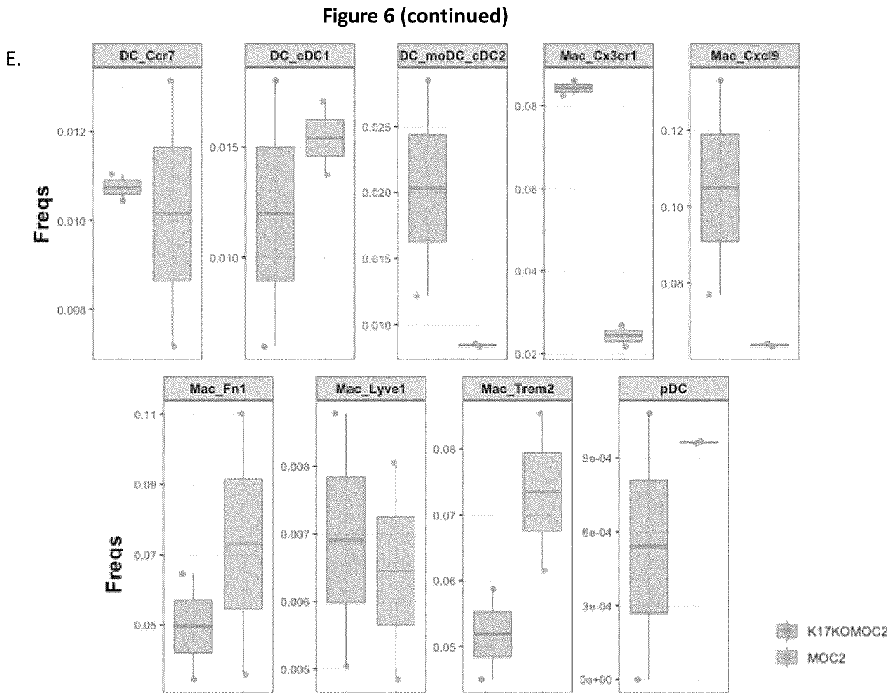

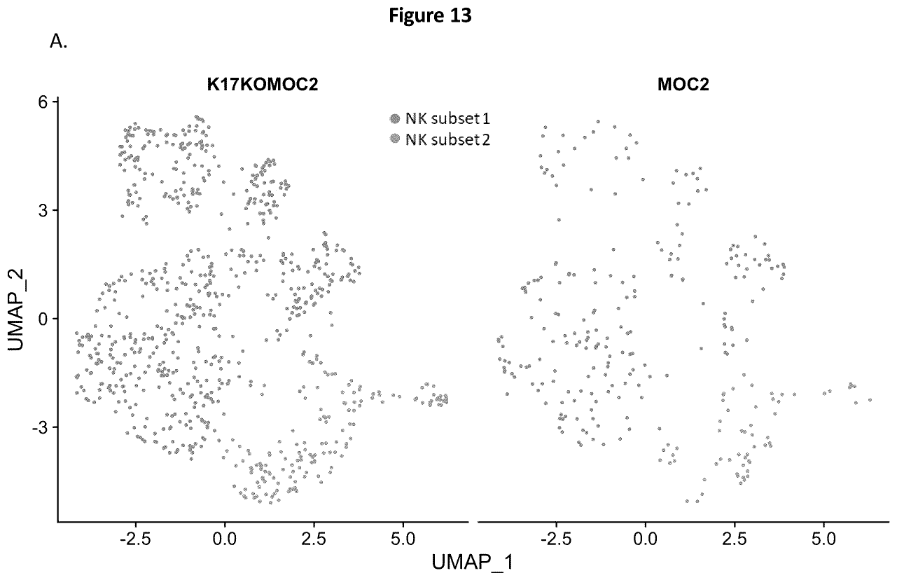

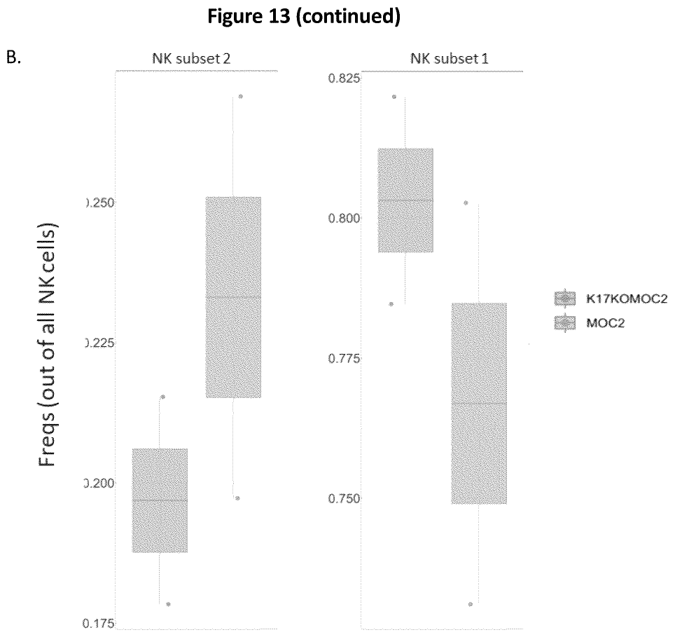

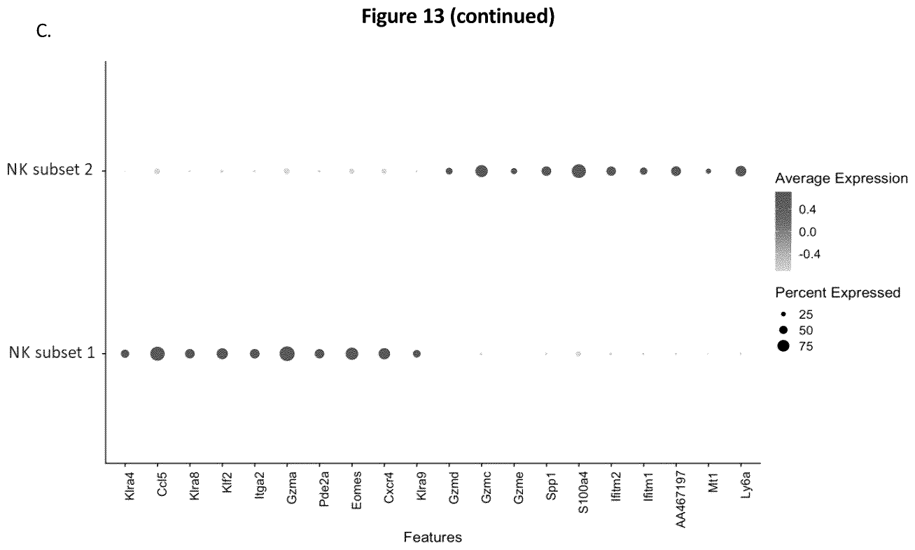

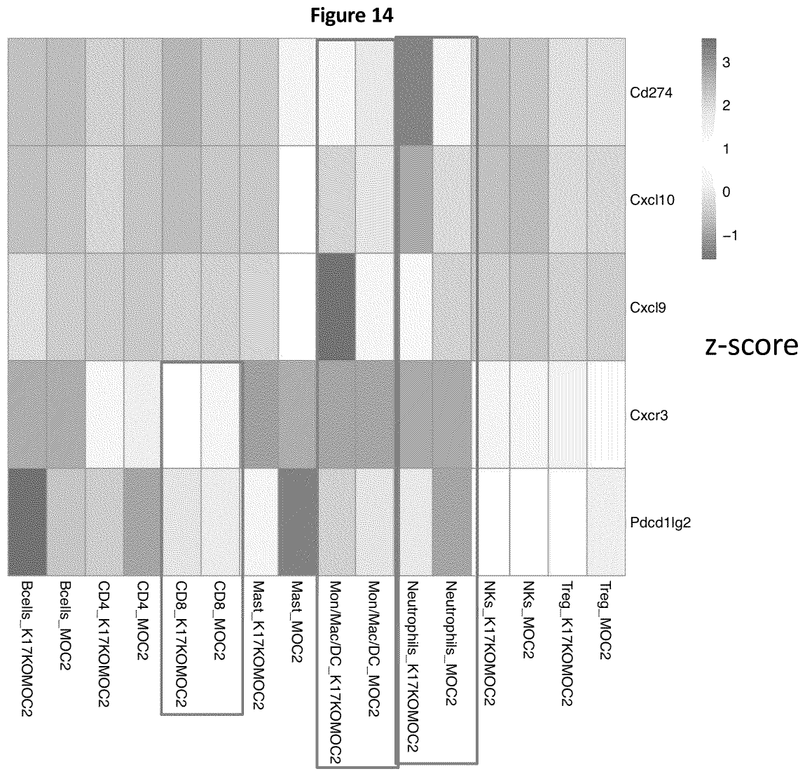

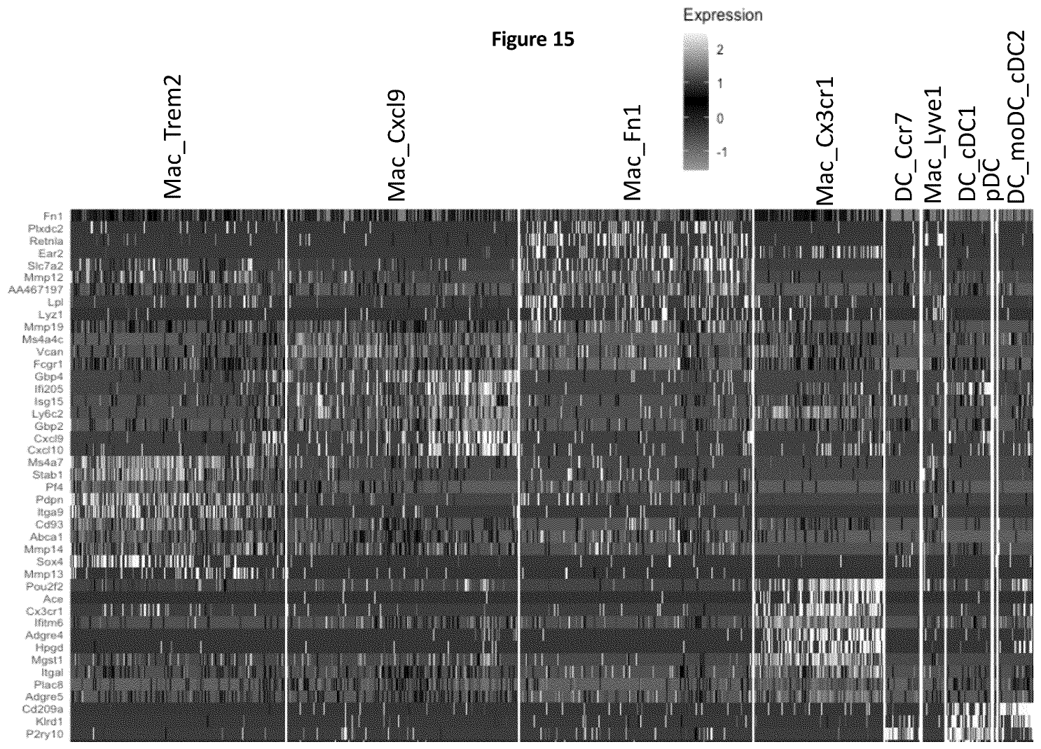

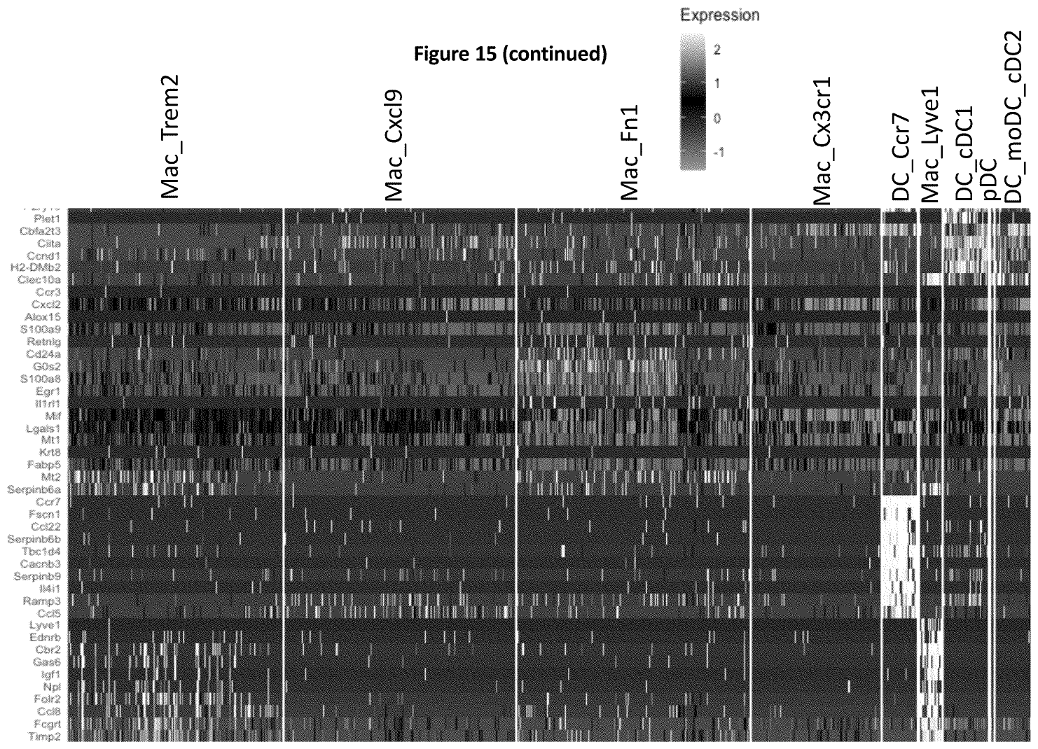

Although we observed partial abrogation of the immune-mediated anti-tumor effect against K17KO MOC2 tumor growth with anti-CXCR3 blocking antibody, there was still a significant growth delay of K17KO MOC2 tumors in CXCR3-blocked mice compared to parental MOC2 tumors (FIG. 3C). These results prompted us to use scRNA-Seq to compare in-depth the immune landscapes of K17KO MOC2 tumors prior to complete regression to that of parental MOC2 tumors. ScRNA-Seq analysis of CD45+ cells between K17KO MOC2 and MOC2 tumors revealed a higher abundance of myeloid cells in MOC2 tumors (compared to K17KO tumors), with neutrophils as the most abundant immune cell-types compared to lymphoid cells (T and B cells) and NK cells (FIG. 6A-C). We observed increases in all 3 major T cell subsets (CD4+, CD8+ T cells and regulatory T cells Treg) with a much higher CD4/Treg and CD8/Treg ratios in the K17KO MOC2 tumors. Subset analysis of NK cells led to identification of two NK cell subsets (Supplemental FIG. 7A). NK cell subset 1 expresses Eomesodermin (Eomes), a crucial transcription factor required for NK cell maturation (32), as well as Ly49 receptors (Klra4, Klra8, Klra9) and integrin CD49b (Itga2), which are markers for mature NK cells (FIG. 13B-C). The NK cells from K17KO MOC2 tumors are skewed towards a more mature phenotype (FIG. 13B-C), suggesting a tumor microenvironment that supports NK cell maturation. Analysis of CXCR3 expression on immune cell subsets (FIG. 14) showed that CXCR3 is more abundantly expressed in CD8+ T cells from K17KO MOC2 tumor, but no difference was observed for other T cell and NK subsets. These data indicate that the increased CD8+ T cell infiltration in K17KO MOC2 cells could be due to increased CXCR3 expression on CD8+ T cells. Given reported heterogeneity of myeloid cells in the tumor microenvironment, we further clustered and identified 4 DC subsets (including pDCs) and 5 macrophage subsets including 1 subset expressing CXCL9 (FIG. 6D, FIG. 15). Interestingly, we saw an opposite trend of enrichment of cDC1 and cDC2 subsets in MOC2 vs. K17KO MOC2 tumors. While cDCI was slightly decreased in frequencies in K17KO MOC2 tumors, cDC2 that expressed monocyte gene signatures were more abundant in K17KO MOC2 samples. Moreover, we observed diversity in macrophage subsets, with M1-like subsets (Mac_CXCL9, Mac_CX3CR1), frequencies of which were increased, and M2-like subset (Mac_Trem2, Mac_Fn1) frequencies of which were decreased in K17KO MOC2 tumors (FIG. 6E). Together, the data show a potentially multifaceted mechanism by which K17 mediates immune evasion in MOC2 tumors.

### Discussion

K17 has been reported as a negative prognostic marker in breast cancer, oral cancer, cervical cancer and ovarian cancer (7, 9-11, 33). In addition, K17 identifies with the most lethal molecular subtype of pancreatic cancer (34). However, how K17 contributes to cancer pathogenesis and worse prognosis is not fully understood. Our analysis of HNSCC tissue microarray, together with our mouse data in this report, provide new insight in the role of K17 in immune evasion and its contribution to cancer pathogenesis. In our TMA data as well as TCGA data analyses, there are still a large number of patients who had low K17 expression that also had low level of CD8 infiltrating T cells, suggesting the overexpression of K17 is just one of many mechanisms that mediates immune evasion by cancers.

Despite being a negative prognostic marker in cancer, the role of K17 in metastasis is more controversial. In a pancreatic cancer model, Zeng et al. have shown that K17 acts as a tumor suppressor and inhibited cancer cell migration and invasion in vitro. By inhibiting K17 in pancreatic cancer cells, they observed increased tumor growth in immunodeficient mice (35). A more recent study by Escobar-Hoyos's group, on the other hand, showed K17 solubilization and nuclear localization enhances tumor growth and metastatic potential using an isogenic murine PDAC model (36). Both of these studies were performed in immunodeficient mice, where the effect of immune response had been excluded. To further investigate the role of K17 in cancer metastasis, an immunocompetent model should be considered.

When we knocked out K17 from MOC2 tumors, we found they could still grow aggressively in immunodeficient mice, indicating K17 was not necessary for their tumorigenicity, but it was important for establishing their growth in immunocompetent mice. Despite upregulated IFNg response in K17KO MOC2 tumors growing in C57BL/6 mice, we also found upregulated PD-L1 and CTLA4 in these tumors, suggesting higher CTLA4 and PD-L1 expression may be a result from K17KO MOC2 tumors evading immune response and supporting their persistent growth in vivo. The most clinically significant observation with this mouse model was that K17 confers resistance to immunotherapy (FIG. 4). Importantly, we found the same to be true in human head and neck cancer patients (FIG. 5).

In cancer patients receiving anti-PD1 therapies, PD-L1 has been identified as a biomarker predictive of response. However, controversies have arisen using PD-L1 as a reliable marker for ICB response (37-39) with some anti-PDI drugs having been approved for treatment of PD-L1 negative cancers too. The results of our exploratory retrospective analysis of 26 HNSCC patients treated with ICB suggest a strong association between K17 status as determined by immunohistochemistry and clinical benefit from ICB therapy, as well as all investigated time-to-event endpoints. Associated challenges were the heterogenous staining in several cases (FIG. 12). Limitations of this analysis are the small sample size and the retrospective nature of the study with associated lack of comprehensive radiologic assessment. Considering the unmet need for predictive biomarkers of response to ICI in HNSCC and the shortcomings of PD-L1 status in this patient population (40), our work supports further validation studies in larger cohorts and in a prospective setting.

Among the 26 HNSCC patients analyzed, 14 of them had available data for PD-L1 expression level. Eleven of them had high PD-L1 expression, and 3 of them had low PD-L1 expression (Table 2). We did not find a correlation between PD-L1 status and K17 expression level, or a correlation between PD-L1 status and their response to Pembrolizumab (Table 3). More patients should be analyzed for their PD-L1 status in this cohort or in separate cohorts of patients to make a meaningful conclusion. Other recent work (41) showed that PD-L1 expression in macrophages and DCs are higher in the patients who responded to anti-PD1 therapy in breast cancer. Our bulk RNA-Seq data from mouse model showed upregulated PD-L1 RNA expression in K17KO MOC2 tumors (FIGS. 2I and 2K), and our scRNA seq data also showed upregulated PD-L1 expression in myeloid cells (FIG. 14), suggesting the upregulation of PD-L1 could be a result from the elevated level of IFNg in the tumor environment because PD-L1 is a IFNg-responsive gene. Whether K17 plays a direct role in regulating PD-L1 expression on tumors cells requires further investigation.

TABLE 3
Correlation analysis between PD-L1 expression and patient
response or between PD-L1 and K17 expression

| | | Patient response to Pembrolizumab | |
|---|---|---|---|
| PD-L1 and response | | no | yes |
| PD-L1 and K17 expression | High | 6 | 1 |
| | Low | 2 | 0 |

| | |
|---|---|
| p value (Fisher's exact test) | 0.9999 |

K17 expression

| PD-L1 and K17 | | Low | High |
|---|---|---|---|
| PD-L1 expression | High | 1 | 6 |
| | Low | 0 | 2 |

| | |
|---|---|
| p value (Fisher's exact test) | 0.9999 |

MOC2 was chosen because it gives rise to immunologically cold, ICB-unresponsive tumors and, as we predicted based upon our prior studies in the context of papillomaviruses that cause cancer (13), was converted to immunologically hot, ICB-responsive tumors once we knocked out K17 (FIGS. 2E-K, FIG. 4A). Importantly, our clinical studies in human HNC patients (FIGS. 1 and 5), confirmed the relevance of our findings with the MOC2 preclinical model. Nevertheless, other mouse HNC models do exist and may be informative. One other, commonly used mouse HNC model is MOC1. It gives rise to immunologically hot, ICB-responsive tumors and is characterized as having a high mutational burden, high MHC class I expression, and is sensitive to innate immune activation, which may drive its phenotype (17, 42, 43). Our own studies on MOC1 indicate that it does express K17, which was counter-intuitive given the preclinical and clinical data presented in this study. We are now engaged in learning if the immune-suppressive effects of K17 are compromised in MOC1 cells, as this may contribute insights into the mechanism of action of K17. One explanation is that the high mutational burden of MOC1 cells, which is predicted lead to higher numbers of neoantigens, overrides K17's effects.

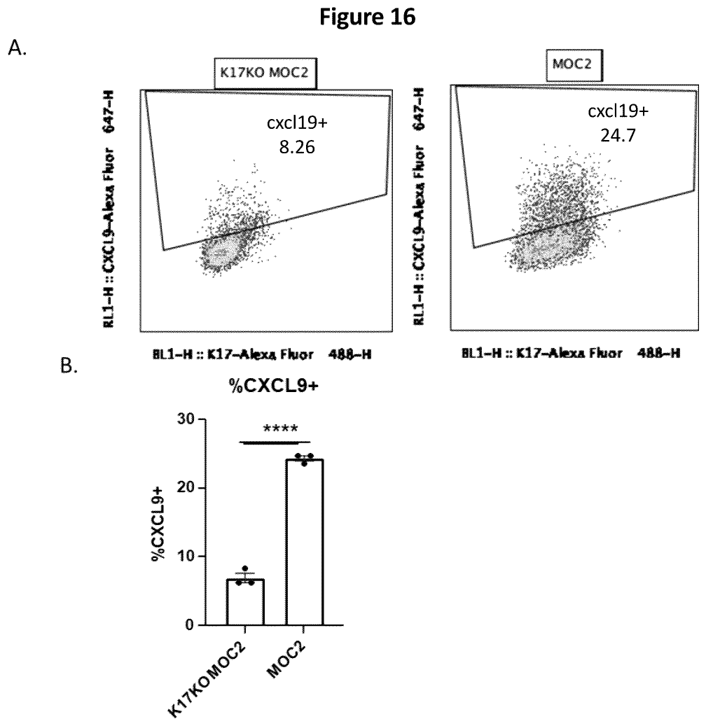

We have previously shown in the mouse papillomavirus (MmuPV1)-induced disease model, that CXCL9/CXCR3 axis was required for successful papilloma regression in K17KO mice (13). In this report, we observed a similar level of CXCL9 upregulation in the K17KO MOC2 tumors. However, when we blocked CXCR3, we only partially rescued the growth of K17KO MOC2 tumors (FIGS. 3C and 3D). These results indicate that, in K17KO MOC2 tumors, CXCL9/CXCR3 is just one of multiple likely chemotaxis signals that contribute to T cell recruitment in K17KO MOC2 tumors. We have previously identified K14+ papilloma cells and macrophages as the major source of CXCL9 in MmuPV1-induced papillomas. Our preliminary in vitro results indicate that K17KO MOC2 cells, upon IFNγ stimulation, produce less CXCL9 than WT MOC2 cells (FIG. 16), which is consistent with what was published by Chung et al (44). These results suggest that K17KO MOC2 tumor cells do not directly contribute to the upregulation of CXCL9 in the tumor microenvironment, rather, other cellular sources of CXCL9, such as macrophages (FIG. 14), may contribute to the CXCL9-enriched environment. Increasing evidence points to a positive role of CXCL9-producing macrophages in fighting cancer and mediating response to ICB therapy. House et al. reported that CXCL9-producing macrophages were associated with more prolonged survival of melanoma patients who received ICB therapy and were essential for ICB efficacy in pre-clinical mouse models (45). Dangaji et al. (46) showed that in variety of human cancers, tumor-derived CCL5 expression and myeloid-derived CXCL9 expression correlated with higher CD8+ T cell infiltration in cancer patients and response to PD-1 blockade therapy. Recent work (47) shows that CXCL9 is the top predictive biomarker for ICB response in HNSCC patients based on gene-panel profiling from the whole tumors from clinical trial data performed by MERCK (48). Therefore, one of the possible mechanism by which K17 downregulates T cell infiltration is by suppressing CXCL9 production in macrophages through tumor cell-macrophage interactions.

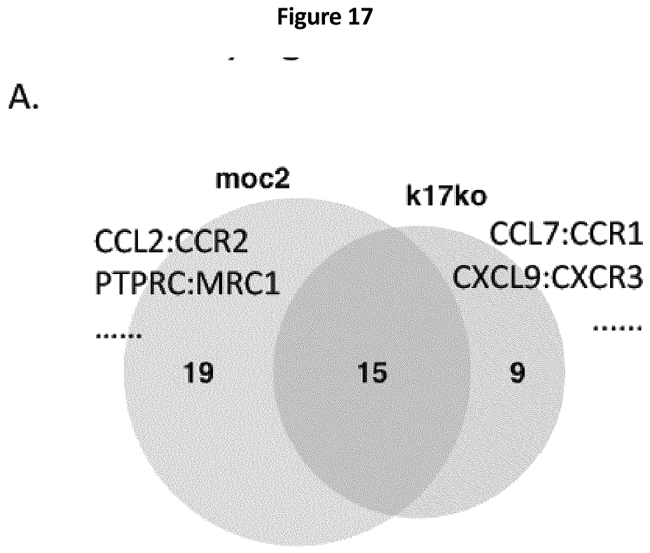

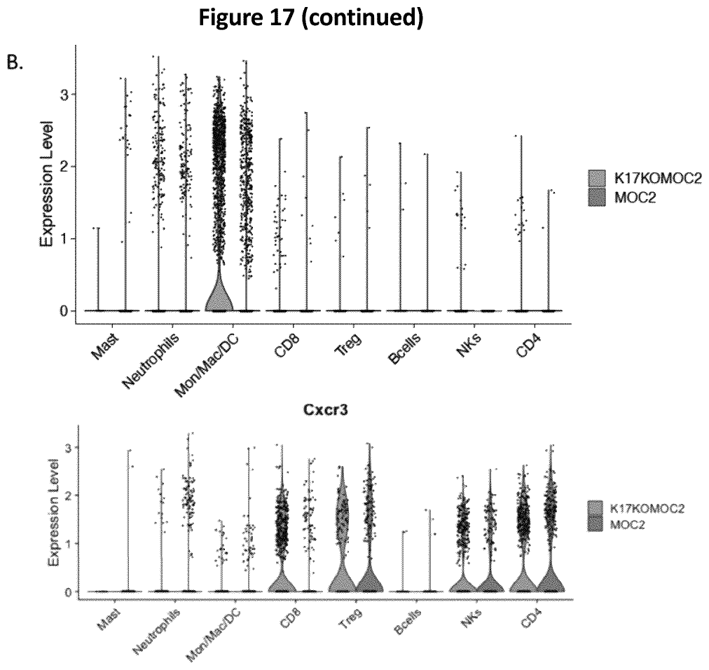

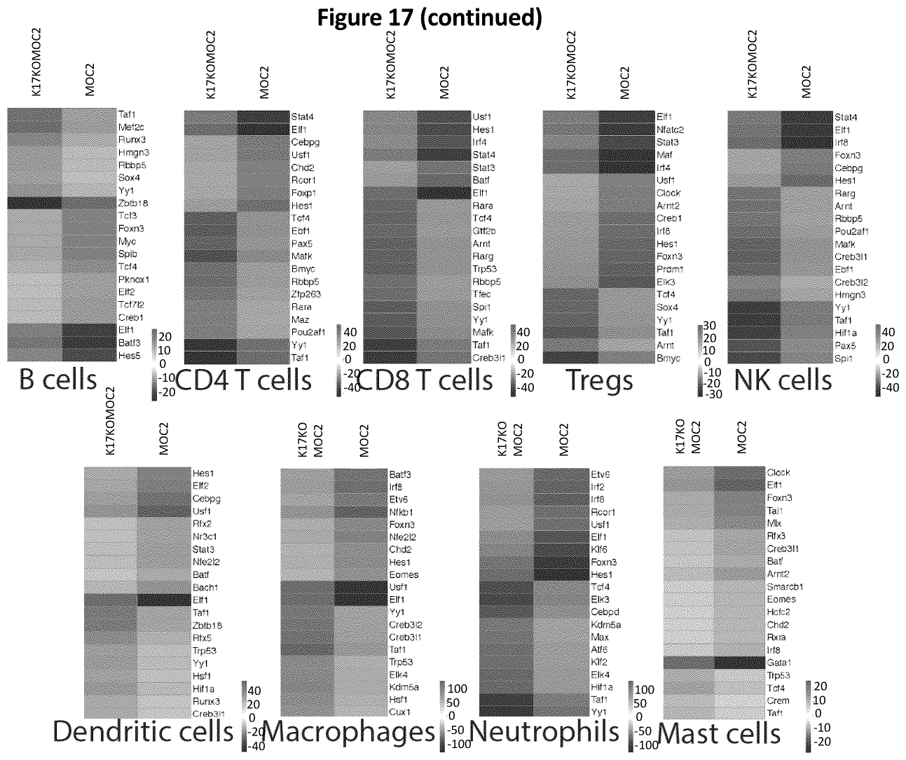

In order to explore other potential mechanisms by which K17KO tumors pose anti-tumoral immune phenotypes, we performed the following two analyses of our scRNA-Seq datasets. First, we inferred ligand-receptor (LR) interactions between different immune cell-types in MOC2 versus K17KO MOC2 tumors by analyzing the scRNA-Seq using CellPhoneDB (29). We identified 15 LR interactions that are shared as well as 19 and 9 unique interactions in MOC2 and K17KO MOC2 tumors, respectively (FIG. 17A). Consistent with our other data (FIG. 2 and FIG. 14), CellPhoneDB identified the CXCL9:CXCR3 interaction between macrophage and T cells was preferentially found in the immune cells infiltrating K17KO MOC2 tumors (FIG. 17B). We also identified the CCL7:CCR1 LR interaction to be uniquely present in K17KO MOC2 tumors. This ligand-receptor interaction is believed to recruit cDC1 dendritic cells to tumors to facilitate ICB response in non-small cell lung cancer (49). The most dominant LR interaction amongst immune cells in MOC2 tumors was CCL2:CCR2, which is known to be pro-tumoral in other tumor types such as breast cancer, hepatocarcinoma and melanoma (50, 51). Second, we sought to identify transcription factor networks differentially expressed in immune cells from MOC2 vs K17KO MOC2 tumors using SCENIC analysis (30) of our scRNA-Seq data sets. We generated a list of TFs that were overexpressed in immune cells from the K17KO MOC2 tumors (FIG. 17C). Interestingly, we found co-expression of leucine zipper ATF-like transcription factor (Batf) and interferon regulatory factor 4 (Irf4) in CD8 T cells. These factors were recently identified to counter T cell exhaustion in the tumor microenvironment (52). Ongoing studies are underway to uncover which of these cell-cell signaling interactions and their downstream target gene expression networks are important in improving the ICB efficacy for K17KO MOC2 tumors, and how the expression of K17 in tumor cells lead to decreased immune response. These studies should provide new insights into how K17 expression facilitates evasion of tumors from immune surveillance and potentially identify new druggable targets that can enhance the efficacy of ICB therapy in patients with non-responsive tumors.

### Methods

### Tissue Microarray

Oropharynx squamous cell carcinoma tissue microarray (TMA) #3 sections were provided by the Wisconsin Head and Neck Cancer SPORE. This TMA section contains 525 cores from 107 oropharynx squamous cell carcinoma. both HPV positive and HPV negative carcinoma: each sample is represented in triplicate 0.6 mm cores. The cancer cores include 171 primary. 207 lymph node metastatic. 6 distant metastatic. and 141 recurrent cancer cores. The tissue microarray (TMA) section was deparaffinized and blocked with 5% Goat serum. Antigens were retrieved in boiling 10 mM citrate buffer for 20 min. Tissues were then washed and stained overnight at 4° C. with anti-K17 (Abcam 109725), anti-CD8 (BioRad MCA351GT), anti-E-Cadherin (Abcam ab231303). Tissues were washed and stained with secondary antibodies conjugated with Alexa 488, Alexa 546 and Alexa 647. Tissues were washed and stained with Hoechst Dye before mounting in ProLong™ Diamond Antifade Mountant. The stained TMA was scanned by Vectra Automated Quantitative Pathology Imaging System at 20× objective. Scanned images were analyzed using inForm software (PerkinElmer). The software was trained using nine scanned images to distinguish tumor compartment (marked by positive E-Cadherin staining) and stromal compartment (marked by negative E-Cadherin staining). Each fluorescence channel was then analyzed within each compartment.

### HNSCC Patient Cohort Evaluated for K17 Expression Level by IHC

Patients diagnosed with squamous cell carcinoma (SCC) of the head and neck region that were treated with immune checkpoint inhibitors (ICI) as part of routine clinical management at the University of Wisconsin-Madison were included in this study. Patient eligibility criteria included pathologic confirmation of SCC, treatment with at least one dose of anti PD-1 drug pembrolizumab, available baseline patient and disease information, and sufficient archival tissue available for analysis. Demographic, clinical, radiographical and treatment data for each patient were obtained from retrospective chart review. Initially, 37 patients were identified, however, only 26 patients had sufficient tissue and data available for analysis.

### Human Study Endpoints

The primary end-point was disease control rate (DCR), i.e. the percentage of patients with radiographic response or stable disease as a result of their therapy. Radiological response assessments were not available for all enrolled patients and we did not wish to exclude patients without radiological reassessment. Therefore, the DCR was investigator-assessed (TL) for all patients with at least one post-treatment scan or evidence of clinical progression after treatment initiation. Progressive disease included radiographic and/or clinical progression. Clinical progression was defined by deterioration of performance status leading to best supportive care/hospice or death in patients without restaging scans available at the time of analysis. Secondary endpoints included progression-free survival (PFS) and overall survival (OS). PFS was defined as the time from initiation of treatment to the time of progression or death due to any cause, while OS was defined as the time from initiation of treatment until time of death or date of last follow up.

### K17 Immunohistochemistry and Quantification

Formalin-fixed, paraffin-embedded tumor specimens from surgical resections were obtained from the archive of the Department of Pathology, sectioned into 4-μm-thick paraffin sections and deparaffinized according to standard procedures before being processed for IHC staining. Deparaffinization was carried out on the instrument, as was heat-induced epitope retrieval in the form of “cell conditioning” with CC1 buffer (Ventana, #950-224), an EDTA based buffer pH 8.4, for 32 minutes at 95° C. IHC for K17 (Anti-Cytokeratin 17, Rabbit Monoclonal, Clone EP1623, dilution 1:100, ab109725, Abcam, Cambridge, United Kingdom) was performed on an automated stainer (Ventana Discovery Ultra BioMarker Platform (Roche, USA)) following the manufacturer's instructions. Semi-quantitative evaluation of K17 expression levels using brightfield microscopy was performed by two surgical pathologists (MBF, JX). Initially, an independent, blinded review was performed. The staining intensity (1+, 2+, 3+), percentage of tumor cells with K17 cytoplasmic staining, and distinct staining patterns were determined. Non-invasive precursor lesions, immune cells, nuclear staining, necrotic cells, and debris were excluded. Cases were categorized into K17 high vs. low defined as >5% strong (3+) cytoplasmic staining intensity of tumor cells observed in the invasive carcinoma component. Cases with strong (3+) cytoplasmic staining intensity in >5% of tumor cells were grouped as high expressors. Cases with low or moderate staining intensity and low percentage of tumor cells with cytoplasmic staining were grouped as low expressors. Some staining patterns (mosaic/basal, perinuclear, golgi expression pattern) were interpreted based on combined IHC and clinicopathologic correlation, and were grouped as low expressors.

### HNSCC Patient Cohort Evaluated for K17 Expression Level by IHC

Patients diagnosed with squamous cell carcinoma (SCC) of the head and neck region that were treated with immune checkpoint inhibitors (ICI) as part of routine clinical management at the University of Wisconsin-Madison were included in this study. Patient eligibility criteria included pathologic confirmation of SCC, treatment with at least one dose of anti PD-1 drug pembrolizumab, available baseline patient and disease information, and sufficient archival tissue available for analysis. Demographic, clinical, radiographical and treatment data for each patient were obtained from retrospective chart review. Initially, 37 patients were identified, however, only 26 patients had sufficient tissue and data available for analysis.

### Human Study Endpoints

The primary end-point was disease control rate (DCR), i.e. the percentage of patients with radiographic response or stable disease as a result of their therapy. Radiological response assessments were not available for all enrolled patients and we did not wish to exclude patients without radiological reassessment. Therefore, the DCR was investigator-assessed (TL) for all patients with at least one post-treatment scan or evidence of clinical progression after treatment initiation. Progressive disease included radiographic and/or clinical progression. Clinical progression was defined by deterioration of performance status leading to best supportive care/hospice or death in patients without restaging scans available at the time of analysis. Secondary endpoints included progression-free survival (PFS) and overall survival (OS). PFS was defined as the time from initiation of treatment to the time of progression or death due to any cause, while OS was defined as the time from initiation of treatment until time of death or date of last follow up.

### K17 Immunohistochemistry and Quantification

Formalin-fixed, paraffin-embedded tumor specimens from surgical resections were obtained from the archive of the Department of Pathology, sectioned into 4-μm-thick paraffin sections and deparaffinized according to standard procedures before being processed for IHC staining. Deparaffinization was carried out on the instrument, as was heat-induced epitope retrieval in the form of “cell conditioning” with CC1 buffer (Ventana, #950-224), an EDTA based buffer pH 8.4, for 32 minutes at 95° C. IHC for K17 (Anti-Cytokeratin 17, Rabbit Monoclonal, Clone EP1623, dilution 1:100, ab109725, Abcam, Cambridge, United Kingdom) was performed on an automated stainer (Ventana Discovery Ultra BioMarker Platform (Roche, USA)) following the manufacturer's instructions. Semi-quantitative evaluation of K17 expression levels using brightfield microscopy was performed by two surgical pathologists (MBF, JX). Initially, an independent, blinded review was performed. The staining intensity (1+, 2+, 3+), percentage of tumor cells with K17 cytoplasmic staining, and distinct staining patterns were determined. Non-invasive precursor lesions, immune cells, nuclear staining, necrotic cells, and debris were excluded. Cases were categorized into K17 high vs. low defined as >5% strong (3+) cytoplasmic staining intensity of tumor cells observed in the invasive carcinoma component. Cases with strong (3+) cytoplasmic staining intensity in >5% of tumor cells were grouped as high expressors. Cases with low or moderate staining intensity and low percentage of tumor cells with cytoplasmic staining were grouped as low expressors. Some staining patterns (mosaic/basal, perinuclear, golgi expression pattern) were interpreted based on combined IHC and clinicopathologic correlation, and were grouped as low expressors.

### Animals

Wildtype C57BL/6 mice and Cas9 knock-in mice (constitutive Cas9-expressing mice; JAX stock #026179) on C57BL/6 background were obtained from Jackson and bred for this study. NOD-scid IL2Rgamma-null (NSG) mice were bred in University of Wisconsin-Madison animal breeding core. All mice were housed in the animal facility in aseptic conditions in micro-isolator cages and experiments carried out under an approved animal protocol. Six-to eight-week-old mice were used for experiments with the same ratio of males and females in each group. For T cell depletion experiment, 100 μg of anti-CD4 (BioXCell, clone GK1.5) and 100 μg of anti-CD8 antibody (BioXCell, clone 2.43) or 100 μg of isotype control (BioXCell, Rat IgG2b, κ) was delivered by intraperitoneal injection twice weekly, starting 1 day before tumor cell injection throughout the study. For detection of CD4 and CD8 depletion, CD8a FITC (Tonbo ebioscience, clone 53-6.7), CD4 PE (Tonbo ebioscience, clone RM4-5) were used for flow cytometry. For CXCR3 blocking experiment, 400 μg of anti-CXCR3 (BioXCell, clone CXCR3-173) or isotype control antibody (BioXCell, Armenian Hamster IgG) was delivered i.p. three times a week, starting 1 day before tumor cell injection, throughout the study.

### Cell Line

MOC2 cells were maintained in F media: 1 part of DMEM+3 parts of F12 media supplemented with 5% FBS, EGF, pen/strep, cholera toxin, insulin, adenine and hydrocortisone. Guide sequences targeting Exon1, Exon4 and Exon5 of mouse KRT17 gene were designed using the Zhang lab's CRISPR guide website: zlab.bio. Annealed oligos containing the designed gRNAs were then ligated into the BsmBI site of LentiCRISPRv.2 and sequence was verified. Lentivirus was made by transfecting 293^{FT} cells with gRNA targeting plasmid, psPAX2 and VSV-g containing plasmids. Lentivirus was then collected 48 hours post transfection and used to infect MOC2 cells. Infected cells were then placed under puromycin selection (5 μg/ml). Pooled cells were verified by immunofluorescence staining and qRT-PCR for K17 expression.

### Flow Cytometry

Subcutaneous tumors were trimmed of surrounding tissues and harvested on ice in PBS. Tumors were cut into 1 mm pieces and digested in 5 mL HBSS supplemented with 5% fetal bovine serum (FBS), 2 mM CaCl2, 2 mM MgCl2, 1 mg/ml collagenase D (Roche) and 200 U/ml DNase I (Roche), at 37° C. for 30 min. Tissues were then homogenized with the back of 1 ml syringe, passed through 0.7 μM filter and washed twice with cold PBS. Blood samples were collected from submandibular bleeding directly into red cell lysis buffer (Tonbo Biosciences) and incubated at room temperature for 10-15 min. Blood cells were then spun down and washed with PBS. Single cell suspensions were then stained with 1 μl Ghost Dye Violet 510 (Tonbo Biosciences) in 1 ml of PBS at 4° C. for 30 min. Samples were then washed with PBS supplemented with 2% FBS, blocked with anti-mouse Fc receptor antibody and stained with cell surface markers. Cells were then washed and fixed with fixation buffer (eBioscience) overnight at 4° C. Cells were washed in PBS supplemented with 2% FBS and analyzed with ThermoFisher Attune. Flow cytometry beads (eBioscience) stained with each antibody were used as single-color controls. A combination of selected antibodies (anti-mouse) was used depending on the purpose of each study: CD45 APC-Cy7 (Biolegend, clone 30-F11), CD8a FITC (Tonbo ebioscience, clone 53-6.7), CD4 PE (Tonbo ebioscience, clone RM4-5), Gr1 PE-Cy5 (Biolegend, clone RB6-8C5), F4/80 BV421 (Biolegend clone BM8), CD11b BV605 (Biolegend, clone M1/70), CD11c PE-Cy7 (Biolegend, clone N418), NKp46 BV711 (Biolegend, clone 29A1.4).

### Immunofluorescent Staining

Tumors were cut in half and embedded in optimal cutting temperature compound (OCT) and frozen on dry ice before storing at −80° C. Frozen tissues were the sectioned (5 microns thick) using a cryostat. Tissue slides were fixed in cold methanol in −20° C. for 10 min, washed with PBS+0.01% Triton X-100, then pure PBS, blocked with 5% goat serum at room temperature for 1 hour, and stained with purified primary antibody at 4° C. overnight. Tissues were then washed with PBS three times, stained with secondary antibodies at room temperature for one hour, counterstained with Hoechst Dye and mounted in Prolong mounting media (Thermo Fisher Scientific). The following antibodies were used for detecting mouse antigens by immunofluorescent staining: CD4 (eBioscience, clone RM4-5), CD8 (eBioscience, clone 53-6.7), K14 (eBioscience, polyclonal Cat #PA5-16722), K17 (provided by Pierre A Coulombe [57]), Goat anti-rabbit AlexaFluor647 (Molecular Probes), Goat anti-rat AlexaFluor 488 (Molecular Probes).

### RNA Sequencing

Fresh tumors were snap frozen in liquid nitrogen, placed into tissueTUBE (Covaris) and pulverized Cryoprep Pulverizer (Covaris). Total RNA was isolated by addition of 1 ml or TRizol (Thermo Fisher Scientific) using RNA-binding columns (Qiagen RNA isolation kit). On column-bound RNA was treated with RQ1 RNase-free DNase (Promega) for 30 min at room temperature, washed with washing buffers, and eluted in RNase-free water. Pooled libraries were sequenced on Illumina NovaSeq 6000. RNA-Seq analysis was done using R and Bioconductor analysis framework. RNA short reads were preprocessed using FastQC (19) to screen for adapter sequence contamination and per-base and per-read quality assessment and then mapped to mouse genome mm10 using subread-align v1.5.3 (20). Short reads overlapping with gene annotation (NCBI RefSeq) were annotated using featureCount (21) for downstream analysis. Differential expressed genes were called with log 2FC cutoff 2, and FDR-adjusted p-value<0.05 using linear model analysis (function voom from limma package) (22) with scaling normalization factors estimated using edgeR (23). Heatmap of DE genes were generated using ComplexHeatmap package (24). Gene set enrichment analysis (GSEA 3.0) was done with genes that have a human homolog (ENSEMBL).

### Single Cell RNA Sequencing

Tumors were collected and sorted for 150,000 live CD45+ cells per sample. Duplicate tumors were collected for each genotype. Around 6000 cells per sample were captured for library preparation, and sequenced on Illumina NovaSeq at the UW-Madison Biotechnology Center. Raw reads were aligned to the mm10 reference genome together with UMI (unique molecular identifier) counting using the Cell Ranger pipeline (v3) from 10× Genomics. Data was filtered using DoubletFinder (25) to remove potential doublets. Further filtering includes only the cells with low mitochondria contents (<=10%) and more than 200 genes covered by the mapping. To integrate the scRNA-Seq, we used a fuzzy clustering-based integration method (Harmony method) (26) to account for potential technical variance across samples. Downstream analysis for all CD45+ cells and for only myeloid cells were based on Seurat single-cell analysis package (27) including: principal component analysis with standard deviation saturation elbow plot to select the optimal number of principal components, graph-based clustering using FindCluster with different resolution from 0.1 to 2 to justify the number of clusters based on representative markers overlaid in the hierarchical tree across different resolution (clustree R package), differentially expression analysis using MAST (28) implemented in Seurat with the cutoff average log 2FC 0.25, and at least 25% of cell expressed the markers. Visualization with heatmap, DotPlot, and violin plot was done using Seurat in R and Bioconductor platform.

To infer ligand/receptor interactions between different immune cells in MOC2 vs. MOC2K17KO tumors, we used CellphoneDB version 2.1.7 (29) with the default parameters. Only interactions with p value<0.05 from the permutation test were considered further for analysis. The list of interactions is shown in Supplemental Tables 4 and 5. We used Single-Cell Regulatory Network Inference and Clustering method, pySCENIC to infer transcription factor gene regulatory networks (30). The regulons were identified from co-expression of transcription factors and their target genes from the RCisTarget database (https://github.com/aertslab/RcisTarget). We ran pySCENIC version 0.11.2 using default parameters. We then used a generalized linear model to identify top regulons that are differentially expressed in MOC2 and MOCK17KO (FDR-corrected p values<0.05) tumors in different immune cell-types based on the AUC scores estimated from pySCENIC.

### qRT-PCR

500 ng of RNA from each sample were used for cDNA synthesis using Quantitect reverse transcription kit (Qiagen). SYBR Green or TaqMan probe were then used for quantitative PCR performed on ABI 7900HT, all gene expression levels were normalized to GAPDH. The following primers were used for SYBR Green detection of mouse gene expressions:

GAPDH forward
(SEQ ID: 1)
5′-CATGGCCTTCCGTGTTCCTA-3′;
GAPDH reverse
(SEQ ID: 2)
5′-GCGGCACGTCAGATCCA-3′;
CXCL9 forward
(SEQ ID: 3)
5′-TCCTCTTGGGCATCATCTTCC-3′;
CXCL9 reverse
(SEQ ID: 4)
5′-TTTGTAGTGGATCGTGCCTCG-3′;
CXCL10 forward
(SEQ ID: 5)
5′-CCAAGTGCTGCCGTCATTTTC-3′;
CXCL10 reverse
(SEQ ID: 6)
5′-GGCTCGCAGGGATGATTTCAA-3′;
CXCL11 forward
(SEQ ID: 7)
5′-GGCTTCCTTATGTTCAAACAGGG-3′;
CXCL11 reverse
(SEQ ID: 8)
5′-GCCGTTACTCGGGTAAATTACA-3′;
PD-L1 forward
(SEQ ID: 9)
5′-CCAGCCACTTCTGAGCATGA-3′;
PD-L1 reverse
(SEQ ID: 10)
5′-CTTCTCTTCCCACTCACGGG-3′;
IFNg forward
(SEQ ID: 11)
5′-ACAATGAACGCTACACACTGCAT-3′;
IFNg reverse
(SEQ ID: 12)
5′-TGGCAGTAACAGCCAGAAACA-3′.

The following TaqMan probes (Thermo Fisher Scientific) were used for K17 expression measurement: GAPDH (Mm99999915_g1); K17 (Mm00495207_m1).

### Statistics

All statistical analyses for animal studies were done with Graphpad Prism. Two-way ANOVA was used for statistical comparison when two variables (tumor growth time and genotype) were involved. When some mice were found dead before the endpoint of study, a mixed-effects model (REML) was used to handle missing values. For single variable experiments, t test or one-way ANOVA was used for statistical comparison as indicated. For survival analyses, log-rank test was used for statistical comparison.

For survival analysis for human TMA and TCGA data, we used the Kaplan-Meier method with right censoring for testing tumor K17 expression and overall survival. We used the Survival package (version 3.2-13) for this analysis. A log-rank test was used to evaluate survival differences between low-and high-groups based on 25^{th} percentile top and bottom of expression values. Two-sided p values<0.05 were considered significant.

The association between clinical response to Pembrolizumab and K17 expression was tested using the Fisher exact test. The PFS and OS outcomes were estimated using the Kaplan-Meier method with appropriate censoring, and the log-rank test was used to investigate differences between groups. Two-sided p<0.05 was considered significant. There were some variations in the timing and interval of radiological assessment given the retrospective nature of this analysis. Therefore, DCR was investigator-assessed based on available imaging and clinical data. Results from this retrospective study should mainly be considered exploratory, so no correction for multiple testing was applied. Statistical analysis was performed using SPSS Statistics version 24 (IBM Corp).

### Example 2: Ligand-Receptor Binding Assays Reveal Differentially Regulated Markers in K17KO MOC2 Tumors and WT MOC2 Tumors

This Example is directed to demonstrating the interactions between ligands and receptors in K17 knockout MOC2 tumors (Table 4) and WT MOC2 tumors (Table 5). These markers were identified using a ligand-receptor binding assay from MOC2 tumors collected from K17 KO and WT models.

The ligands and receptors identified in Tables 4 and 5 can be used as additional markers in addition to K17 for the ability to select a subject that has a tumor that is or is not responsive to immunotherapies. For example, the markers that are listed in Table 4 and Table 5, which are ligand-receptor interactions that are differentially regulated and may contribute to K17 mode of action, can be used as additional markers with K17, and detection of the K17 and additional marker allows for the patients to be classified as having a tumor that is or is not responsive to immunotherapies. The ligand and receptor interactions uniquely detected in K17KO tumors (Table 4) can be used in combination with low K17 expression to predict responsiveness to immunotherapy. The unique ligand-receptors interactions predictive of responsiveness to immunotherapy include the following markers: IFNγ, CXCL10, CXCL11, PD-L1, CCL15, CCL23, CCL7, CXCL9, ENTPD1, SIRPG, ADORA2A, CCR1, CD47, CXCR3, DPP4, and/or FCGR2A. In particular the ligand-receptor pairs of markers found in Table 4 below may be detected in combination and further in combination with low K17 expression to be indicative of responsiveness to immunotherapy. The ligand and receptor interactions uniquely detected in WT tumors (Table 5) can be used in combination with high K17 expression to predict non-responsiveness to immunotherapy. Those markers include, but are not limited to, CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, and/or SPN. In particular, the ligand-receptor pairs of markers found in Table 5 below may be detected in combination with each other and further in combination with high K17 expression to be indicative of non-responsiveness to immunotherapy.

TABLE 4
Unique ligand-receptor interactions identified in K17KO MOC2 tumors

| ligand_k17ko | receptor_k17ko | sender_k17ko | receiver_k17ko | sigmean_k17ko | pval_k17ko |
|---|---|---|---|---|---|
| CCL15 | CCR1 | Neutrophils | DCCcr7 | 2.141 | 0 |
| CCL23 | CCR1 | Neutrophils | DCCcr7 | 2.141 | 0 |
| CCL15 | CCR1 | pDC | DCcDC1 | 1.671 | 0.011 |
| CCL23 | CCR1 | pDC | DCcDC1 | 1.671 | 0.011 |
| CCL7 | CCR1 | DCCcr7 | Mast | 1.641 | 0 |
| CCL7 | CCR1 | MacLyve1 | Mast | 1.638 | 0 |
| CCL7 | CCR1 | MacCxcl9 | Mast | 1.636 | 0 |
| CCL7 | CCR1 | MacCx3cr1 | Mast | 1.614 | 0 |
| CCL7 | CCR1 | DCmoDCcDC2 | Mast | 1.597 | 0 |
| CCL15 | CCR1 | DCCcr7 | MacTrem2 | 1.452 | 0 |
| CCL23 | CCR1 | DCCcr7 | MacTrem2 | 1.452 | 0 |
| CCL15 | CCR1 | MacLyve1 | MacTrem2 | 1.449 | 0 |
| CCL23 | CCR1 | MacLyve1 | MacTrem2 | 1.449 | 0 |
| CCL15 | CCR1 | MacCxcl9 | MacTrem2 | 1.447 | 0 |
| CCL23 | CCR1 | MacCxcl9 | MacTrem2 | 1.447 | 0 |
| CCL15 | CCR1 | DCcDC1 | MacTrem2 | 1.444 | 0 |
| CCL23 | CCR1 | DCcDC1 | MacTrem2 | 1.444 | 0 |
| CCL15 | CCR1 | MacFn1 | MacTrem2 | 1.442 | 0 |
| CCL23 | CCR1 | MacFn1 | MacTrem2 | 1.442 | 0 |
| CCL15 | CCR1 | DCCcr7 | MacLyve1 | 1.432 | 0 |
| CCL23 | CCR1 | DCCcr7 | MacLyve1 | 1.432 | 0 |
| CCL15 | CCR1 | MacLyve1 | MacLyve1 | 1.429 | 0 |
| CCL23 | CCR1 | MacLyve1 | MacLyve1 | 1.429 | 0 |
| CCL15 | CCR1 | MacCxcl9 | MacLyve1 | 1.427 | 0 |
| CCL23 | CCR1 | MacCxcl9 | MacLyve1 | 1.427 | 0 |
| CCL15 | CCR1 | MacCx3cr1 | MacTrem2 | 1.425 | 0 |
| CCL23 | CCR1 | MacCx3cr1 | MacTrem2 | 1.425 | 0 |
| CCL15 | CCR1 | DCcDC1 | MacLyve1 | 1.424 | 0 |
| CCL23 | CCR1 | DCcDC1 | MacLyve1 | 1.424 | 0 |
| CCL15 | CCR1 | MacFn1 | MacLyve1 | 1.422 | 0 |
| CCL23 | CCR1 | MacFn1 | MacLyve1 | 1.422 | 0 |
| CCL15 | CCR1 | DCCcr7 | MacFn1 | 1.408 | 0 |
| CCL15 | CCR1 | DCmoDCcDC2 | MacTrem2 | 1.408 | 0 |
| CCL23 | CCR1 | DCCcr7 | MacFn1 | 1.408 | 0 |
| CCL23 | CCR1 | DCmoDCcDC2 | MacTrem2 | 1.408 | 0 |
| CCL15 | CCR1 | MacCx3cr1 | MacLyve1 | 1.405 | 0 |
| CCL15 | CCR1 | MacLyve1 | MacFn1 | 1.405 | 0 |
| CCL23 | CCR1 | MacCx3cr1 | MacLyve1 | 1.405 | 0 |
| CCL23 | CCR1 | MacLyve1 | MacFn1 | 1.405 | 0 |
| CCL15 | CCR1 | MacCxcl9 | MacFn1 | 1.403 | 0 |
| CCL23 | CCR1 | MacCxcl9 | MacFn1 | 1.403 | 0 |
| CCL15 | CCR1 | DCcDC1 | MacFn1 | 1.4 | 0 |
| CCL23 | CCR1 | DCcDC1 | MacFn1 | 1.4 | 0 |
| CCL15 | CCR1 | MacFn1 | MacFn1 | 1.399 | 0 |
| CCL23 | CCR1 | MacFn1 | MacFn1 | 1.399 | 0 |
| CCL15 | CCR1 | DCmoDCcDC2 | MacLyve1 | 1.388 | 0 |
| CCL23 | CCR1 | DCmoDCcDC2 | MacLyve1 | 1.388 | 0 |
| CCL15 | CCR1 | MacCx3cr1 | MacFn1 | 1.381 | 0 |
| CCL23 | CCR1 | MacCx3cr1 | MacFn1 | 1.381 | 0 |
| CXCL9 | FCGR2A | pDC | DCCcr7 | 1.381 | 0 |
| CCL15 | CCR1 | DCmoDCcDC2 | MacFn1 | 1.364 | 0 |
| CCL23 | CCR1 | DCmoDCcDC2 | MacFn1 | 1.364 | 0 |
| CXCL9 | FCGR2A | pDC | DCcDC1 | 1.354 | 0 |
| SIRPG | CD47 | NK2 | DCCcr7 | 1.34 | 0 |
| CXCL9 | FCGR2A | DCCcr7 | MacFn1 | 1.334 | 0 |
| CCL15 | CCR1 | Neutrophils | CD4 | 1.317 | 0 |
| CCL15 | CCR1 | Neutrophils | CD8 | 1.317 | 0 |
| CCL23 | CCR1 | Neutrophils | CD4 | 1.317 | 0 |
| CCL23 | CCR1 | Neutrophils | CD8 | 1.317 | 0 |
| CXCL9 | FCGR2A | MacCx3cr1 | MacFn1 | 1.316 | 0 |
| CXCL9 | FCGR2A | MacCxcl9 | MacFn1 | 1.31 | 0 |
| CXCL9 | FCGR2A | MacFn1 | MacFn1 | 1.303 | 0 |
| SIRPG | CD47 | NK1 | Neutrophils | 1.284 | 0 |
| CXCL9 | FCGR2A | MacLyve1 | MacFn1 | 1.282 | 0 |
| SIRPG | CD47 | NK1 | DCCcr7 | 1.282 | 0 |
| CXCL9 | FCGR2A | DCCcr7 | MacTrem2 | 1.259 | 0 |
| SIRPG | CD47 | NK1 | MacTrem2 | 1.258 | 0 |
| CXCL9 | FCGR2A | DCCcr7 | MacLyve1 | 1.251 | 0 |
| CXCL9 | FCGR2A | DCmoDCcDC2 | MacFn1 | 1.251 | 0 |
| CXCL9 | FCGR2A | DCcDC1 | MacFn1 | 1.247 | 0 |
| SIRPG | CD47 | NK1 | MacFn1 | 1.245 | 0 |
| CXCL9 | FCGR2A | MacCx3cr1 | MacTrem2 | 1.241 | 0 |
| CXCL9 | FCGR2A | MacCxcl9 | MacTrem2 | 1.234 | 0 |
| CXCL9 | FCGR2A | MacCx3cr1 | MacLyve1 | 1.233 | 0 |
| CXCL9 | FCGR2A | MacFn1 | MacTrem2 | 1.228 | 0 |
| SIRPG | CD47 | CD8 | Neutrophils | 1.228 | 0 |
| CXCL9 | FCGR2A | MacCxcl9 | MacLyve1 | 1.226 | 0 |
| CXCL9 | FCGR2A | MacFn1 | MacLyve1 | 1.219 | 0 |
| CXCL9 | FCGR2A | MacLyve1 | MacTrem2 | 1.207 | 0 |
| SIRPG | CD47 | CD8 | MacTrem2 | 1.201 | 0 |
| CXCL9 | FCGR2A | MacLyve1 | MacLyve1 | 1.198 | 0 |
| SIRPG | CD47 | DCcDC1 | Neutrophils | 1.19 | 0.001 |
| SIRPG | CD47 | MacFn1 | Neutrophils | 1.177 | 0 |
| CXCL9 | FCGR2A | DCmoDCcDC2 | MacTrem2 | 1.176 | 0 |
| SIRPG | CD47 | Treg | DCCcr7 | 1.173 | 0.001 |
| CXCL9 | FCGR2A | DCcDC1 | MacTrem2 | 1.171 | 0 |
| SIRPG | CD47 | MacLyve1 | MacLyve1 | 1.169 | 0.022 |
| CXCL9 | FCGR2A | DCmoDCcDC2 | MacLyve1 | 1.167 | 0 |
| SIRPG | CD47 | MacCxcl9 | Neutrophils | 1.165 | 0 |
| SIRPG | CD47 | MacFn1 | MacLyve1 | 1.165 | 0 |
| SIRPG | CD47 | CD4 | Neutrophils | 1.164 | 0 |
| CXCL9 | FCGR2A | DCcDC1 | MacLyve1 | 1.163 | 0 |
| SIRPG | CD47 | DCcDC1 | MacTrem2 | 1.163 | 0.001 |
| SIRPG | CD47 | DCmoDCcDC2 | Neutrophils | 1.161 | 0.001 |
| SIRPG | CD47 | MacCx3cr1 | Neutrophils | 1.155 | 0 |
| SIRPG | CD47 | MacLyve1 | MacTrem2 | 1.154 | 0.015 |
| SIRPG | CD47 | MacCxcl9 | MacLyve1 | 1.153 | 0.001 |
| SIRPG | CD47 | CD4 | MacLyve1 | 1.151 | 0.001 |
| SIRPG | CD47 | MacFn1 | MacTrem2 | 1.151 | 0 |
| SIRPG | CD47 | DCCcr7 | Neutrophils | 1.15 | 0.012 |
| SIRPG | CD47 | DCmoDCcDC2 | MacLyve1 | 1.149 | 0.031 |
| SIRPG | CD47 | MacCx3cr1 | MacLyve1 | 1.143 | 0.001 |
| SIRPG | CD47 | MacLyve1 | MacFn1 | 1.142 | 0.027 |
| SIRPG | CD47 | MacCxcl9 | MacTrem2 | 1.139 | 0 |
| SIRPG | CD47 | MacFn1 | MacFn1 | 1.138 | 0 |
| SIRPG | CD47 | CD4 | MacTrem2 | 1.137 | 0 |
| SIRPG | CD47 | DCCcr7 | MacLyve1 | 1.137 | 0.044 |
| SIRPG | CD47 | DCmoDCcDC2 | MacTrem2 | 1.135 | 0.019 |
| SIRPG | CD47 | MacCx3cr1 | MacTrem2 | 1.129 | 0 |
| SIRPG | CD47 | MacCxcl9 | MacFn1 | 1.126 | 0 |
| SIRPG | CD47 | CD4 | MacFn1 | 1.124 | 0 |
| SIRPG | CD47 | DCCcr7 | MacTrem2 | 1.123 | 0.046 |
| SIRPG | CD47 | DCmoDCcDC2 | MacFn1 | 1.122 | 0.036 |
| SIRPG | CD47 | MacCx3cr1 | MacFn1 | 1.116 | 0.001 |
| CCL15 | CCR1 | MacCxcl9 | Neutrophils | 1.088 | 0.022 |
| CCL23 | CCR1 | MacCxcl9 | Neutrophils | 1.088 | 0.022 |
| CXCL9 | FCGR2A | Neutrophils | DCCcr7 | 1.088 | 0 |
| CCL15 | CCR1 | MacFn1 | Neutrophils | 1.083 | 0.043 |
| CCL23 | CCR1 | MacFn1 | Neutrophils | 1.083 | 0.043 |
| SIRPG | CD47 | Neutrophils | Neutrophils | 1.083 | 0 |
| ENTPD1 | ADORA2A | Mast | Neutrophils | 1.007 | 0 |
| CXCL9 | CXCR3 | CD4 | MacFn1 | 0.844 | 0 |
| CXCL9 | DPP4 | pDC | pDC | 0.814 | 0.005 |
| CCL7 | CCR1 | DCCcr7 | MacFn1 | 0.812 | 0.035 |
| CCL7 | CCR1 | MacCxcl9 | MacFn1 | 0.806 | 0 |
| CCL7 | CCR1 | MacCx3cr1 | MacFn1 | 0.785 | 0.002 |
| CXCL9 | CXCR3 | CD4 | MacTrem2 | 0.768 | 0 |
| CXCL9 | CXCR3 | CD4 | MacLyve1 | 0.76 | 0 |
| CXCL9 | DPP4 | Bcells | MacFn1 | 0.756 | 0 |
| CXCL9 | DPP4 | CD4 | MacFn1 | 0.753 | 0 |

TABLE 5
Unique ligand-receptor interactions identified in WT MOC2 tumors

| ligand_moc2 | receptor_moc2 | sender_moc2 | Receiver_Moc2 | Sigmean_moc2 | pval_moc2 |
|---|---|---|---|---|---|
| CCL2 | CCR2 | Mast | NK1 | 2.094 | 0 |
| CCL2 | CCR2 | Mast | NK2 | 2.029 | 0 |
| PTPRC | MRC1 | NK2 | MacTrem2 | 2.015 | 0 |
| PTPRC | MRC1 | Treg | MacTrem2 | 2.012 | 0 |
| PTPRC | MRC1 | CD4 | MacFn1 | 2.009 | 0 |
| PTPRC | MRC1 | CD4 | MacTrem2 | 2.002 | 0 |
| PTPRC | MRC1 | NK1 | MacTrem2 | 1.996 | 0 |
| PTPRC | MRC1 | CD4 | MacLyve1 | 1.984 | 0 |
| CCL2 | CCR2 | Mast | DCCcr7 | 1.948 | 0 |
| PTPRC | MRC1 | Neutrophils | MacTrem2 | 1.944 | 0 |
| PTPRC | MRC1 | CD8 | MacFn1 | 1.937 | 0 |
| PTPRC | MRC1 | CD8 | MacTrem2 | 1.929 | 0 |
| PTPRC | MRC1 | CD8 | MacLyve1 | 1.911 | 0 |
| CCL2 | CCR2 | Mast | MacFn1 | 1.908 | 0 |
| CCL2 | CCR2 | Mast | MacTrem2 | 1.876 | 0 |
| CCL2 | CCR2 | Mast | MacLyve1 | 1.86 | 0 |
| PTPRC | MRC1 | Bcells | MacFn1 | 1.777 | 0 |
| PTPRC | MRC1 | Bcells | MacTrem2 | 1.769 | 0 |
| PTPRC | MRC1 | Bcells | MacLyve1 | 1.752 | 0 |
| PTPRC | MRC1 | MacFn1 | MacTrem2 | 1.706 | 0 |
| PTPRC | MRC1 | DCCcr7 | MacFn1 | 1.702 | 0 |
| PTPRC | MRC1 | pDC | MacTrem2 | 1.696 | 0 |
| PTPRC | MRC1 | DCCcr7 | MacTrem2 | 1.695 | 0 |
| PTPRC | MRC1 | MacTrem2 | MacTrem2 | 1.691 | 0 |
| PTPRC | MRC1 | DCcDC1 | MacFn1 | 1.689 | 0 |
| PTPRC | MRC1 | DCmoDCcDC2 | MacFn1 | 1.684 | 0 |
| PTPRC | MRC1 | pDC | MacCx3cr1 | 1.684 | 0 |
| PTPRC | MRC1 | DCcDC1 | MacTrem2 | 1.681 | 0 |
| PTPRC | MRC1 | MacCxcl9 | MacFn1 | 1.68 | 0 |
| PTPRC | MRC1 | DCCcr7 | MacLyve1 | 1.677 | 0 |
| PTPRC | MRC1 | DCmoDCcDC2 | MacTrem2 | 1.676 | 0 |
| PTPRC | MRC1 | MacCxcl9 | MacTrem2 | 1.672 | 0 |
| PTPRC | MRC1 | MacCx3cr1 | MacFn1 | 1.669 | 0 |
| PTPRC | MRC1 | MacLyve1 | MacTrem2 | 1.667 | 0 |
| PTPRC | MRC1 | DCcDC1 | MacLyve1 | 1.663 | 0 |
| PTPRC | MRC1 | pDC | pDC | 1.663 | 0.034 |
| PTPRC | MRC1 | MacCx3cr1 | MacTrem2 | 1.661 | 0 |
| PTPRC | MRC1 | DCmoDCcDC2 | MacLyve1 | 1.659 | 0 |
| CCL2 | CCR2 | Mast | pDC | 1.654 | 0 |
| PTPRC | MRC1 | MacCxcl9 | MacLyve1 | 1.654 | 0 |
| CCL3 | CCR5 | Neutrophils | DCCcr7 | 1.65 | 0 |
| SELL | SELPLG | Bcells | CD8 | 1.648 | 0.008 |
| PTPRC | MRC1 | NK2 | Mast | 1.646 | 0 |
| PTPRC | MRC1 | MacCx3cr1 | MacLyve1 | 1.643 | 0 |
| PTPRC | MRC1 | Treg | Mast | 1.642 | 0 |
| PTPRC | MRC1 | CD4 | Mast | 1.632 | 0 |
| PTPRC | MRC1 | NK1 | Mast | 1.627 | 0 |
| CCL4 | CCR5 | Neutrophils | DCCcr7 | 1.617 | 0 |
| SELL | SELPLG | Bcells | CD4 | 1.612 | 0.038 |
| CCL2 | CCR2 | Mast | Mast | 1.591 | 0 |
| PTPRC | MRC1 | Mast | MacTrem2 | 1.579 | 0 |
| PTPRC | MRC1 | Neutrophils | Mast | 1.575 | 0 |
| CCL2 | CCR2 | Mast | CD8 | 1.562 | 0 |
| PTPRC | MRC1 | CD8 | Mast | 1.56 | 0 |
| CCL2 | CCR2 | DCCcr7 | Treg | 1.536 | 0 |
| CCL2 | CCR2 | DCcDC1 | Treg | 1.515 | 0 |
| CCL4 | CCR5 | Mast | DCCcr7 | 1.504 | 0 |
| CCL2 | CCR2 | MacTrem2 | Treg | 1.49 | 0 |
| CCL2 | CCR2 | MacCxcl9 | Treg | 1.486 | 0 |
| CCL2 | CCR2 | DCmoDCcDC2 | Treg | 1.479 | 0 |
| CCL2 | CCR2 | MacCx3cr1 | Treg | 1.47 | 0 |
| CCL2 | CCR2 | DCCcr7 | NK1 | 1.432 | 0 |
| CCL2 | CCR2 | MacLyve1 | Treg | 1.421 | 0 |
| CCL2 | CCR2 | DCcDC1 | NK1 | 1.411 | 0 |
| CCL3 | CCR5 | Neutrophils | CD8 | 1.399 | 0 |
| CCL2 | CCR2 | MacTrem2 | NK1 | 1.386 | 0 |
| CCL2 | CCR2 | MacCxcl9 | NK1 | 1.382 | 0 |
| CCL2 | CCR2 | DCmoDCcDC2 | NK1 | 1.375 | 0 |
| CCL2 | CCR2 | DCCcr7 | NK2 | 1.368 | 0 |
| CCL4 | CCR5 | Neutrophils | CD8 | 1.367 | 0 |
| CCL2 | CCR2 | MacCx3cr1 | NK1 | 1.366 | 0 |
| CCL2 | CCR2 | DCcDC1 | NK2 | 1.346 | 0 |
| CCL2 | CCR2 | MacTrem2 | NK2 | 1.322 | 0 |
| CCL2 | CCR2 | MacCxcl9 | NK2 | 1.318 | 0 |
| CCL2 | CCR2 | MacLyve1 | NK1 | 1.317 | 0 |
| CCL2 | CCR2 | DCmoDCcDC2 | NK2 | 1.311 | 0 |
| CD28 | CD80 | CD4 | Neutrophils | 1.306 | 0 |
| CCL2 | CCR2 | MacCx3cr1 | NK2 | 1.302 | 0 |
| CCL2 | CCR2 | MacFn1 | DCCcr7 | 1.293 | 0 |
| CCL2 | CCR2 | DCCcr7 | DCCcr7 | 1.287 | 0 |
| CCL2 | CCR2 | DCcDC1 | DCCcr7 | 1.265 | 0 |
| CCL4 | CCR5 | MacTrem2 | NK2 | 1.265 | 0.002 |
| CCL4 | CCR5 | Mast | CD8 | 1.254 | 0 |
| CCL2 | CCR2 | MacLyve1 | NK2 | 1.253 | 0 |
| CCL2 | CCR2 | DCCcr7 | MacFn1 | 1.246 | 0 |
| CCL4 | CCR5 | MacCxcl9 | NK2 | 1.246 | 0.004 |
| CCL4 | CCR5 | NK2 | NK2 | 1.244 | 0.031 |
| CCL2 | CCR2 | MacTrem2 | DCCcr7 | 1.24 | 0 |
| CCL2 | CCR2 | MacCxcl9 | DCCcr7 | 1.237 | 0 |
| CCL24 | CCR2 | DCcDC1 | Treg | 1.237 | 0 |
| CCL2 | CCR2 | MacFn1 | MacCx3cr1 | 1.234 | 0 |
| CCL2 | CCR2 | MacFn1 | MacCxcl9 | 1.234 | 0 |
| CCL2 | CCR2 | DCmoDCcDC2 | DCCcr7 | 1.23 | 0 |
| CCL2 | CCR2 | DCCcr7 | MacCxcl9 | 1.228 | 0 |
| CCL2 | CCR2 | DCCcr7 | MacCx3cr1 | 1.227 | 0 |
| CCL24 | CCR2 | DCCcr7 | Treg | 1.227 | 0 |
| CCL4 | CCR5 | NK2 | Treg | 1.226 | 0.048 |
| CCL2 | CCR2 | DCcDC1 | MacFn1 | 1.225 | 0 |
| CCL4 | CCR5 | MacTrem2 | NK1 | 1.222 | 0 |
| CCL2 | CCR2 | MacCx3cr1 | DCCcr7 | 1.22 | 0 |
| CCL2 | CCR2 | DCCcr7 | MacTrem2 | 1.215 | 0 |
| CCL4 | CCR5 | MacCx3cr1 | MacFn1 | 1.214 | 0 |
| CCL4 | CCR5 | MacTrem2 | MacTrem2 | 1.214 | 0 |
| CCL4 | CCR5 | MacCx3cr1 | NK1 | 1.213 | 0 |
| CCL24 | CCR2 | DCmoDCcDC2 | Treg | 1.211 | 0 |
| CCL4 | CCR5 | DCcDC1 | MacFn1 | 1.209 | 0 |
| CCL4 | CCR5 | DCcDC1 | NK1 | 1.208 | 0.003 |
| CCL2 | CCR2 | DCcDC1 | MacCx3cr1 | 1.206 | 0 |
| CCL2 | CCR2 | DCcDC1 | MacCxcl9 | 1.206 | 0 |
| CD28 | CD86 | CD4 | DCcDC1 | 1.206 | 0 |
| CCL4 | CCR5 | MacCxcl9 | MacFn1 | 1.205 | 0 |
| CCL2 | CCR2 | pDC | DCCcr7 | 1.204 | 0 |
| CCL24 | CCR2 | MacCxcl9 | Treg | 1.204 | 0 |
| CCL4 | CCR5 | MacCx3cr1 | MacTrem2 | 1.204 | 0 |
| CCL4 | CCR5 | MacCxcl9 | NK1 | 1.203 | 0 |
| CCL2 | CCR2 | DCCcr7 | MacLyve1 | 1.199 | 0 |
| CCL2 | CCR2 | MacCxcl9 | MacFn1 | 1.196 | 0 |
| CCL24 | CCR2 | MacCx3cr1 | Treg | 1.195 | 0 |
| CCL4 | CCR5 | MacCxcl9 | MacTrem2 | 1.195 | 0 |
| CCL2 | CCR2 | DCcDC1 | MacTrem2 | 1.193 | 0 |
| CCL2 | CCR2 | DCmoDCcDC2 | MacFn1 | 1.189 | 0 |
| CCL4 | CCR5 | DCmoDCcDC2 | MacFn1 | 1.188 | 0.002 |
| CCL4 | CCR5 | DCmoDCcDC2 | NK1 | 1.187 | 0.005 |
| CCL2 | CCR2 | MacCx3cr1 | MacFn1 | 1.18 | 0 |
| CCL4 | CCR5 | DCmoDCcDC2 | MacTrem2 | 1.179 | 0.004 |
| CCL2 | CCR2 | MacCxcl9 | MacCxcl9 | 1.178 | 0 |
| CCL3 | CCR5 | DCcDC1 | NK1 | 1.178 | 0.005 |
| CCL2 | CCR2 | DCcDC1 | MacLyve1 | 1.177 | 0 |
| CCL2 | CCR2 | MacCxcl9 | MacCx3cr1 | 1.177 | 0 |
| CCL2 | CCR2 | MacTrem2 | DCmoDCcDC2 | 1.174 | 0 |
| CCL2 | CCR2 | DCmoDCcDC2 | MacCxcl9 | 1.171 | 0 |
| CCL2 | CCR2 | DCmoDCcDC2 | MacCx3cr1 | 1.17 | 0 |
| CD28 | CD86 | CD4 | MacTrem2 | 1.17 | 0 |
| CCL2 | CCR2 | MacTrem2 | MacTrem2 | 1.169 | 0 |
| CCL2 | CCR2 | MacCxcl9 | MacTrem2 | 1.165 | 0 |
| CCL3 | CCR5 | MacCxcl9 | MacFn1 | 1.163 | 0 |
| CCL3 | CCR5 | MacCxcl9 | NK1 | 1.162 | 0 |
| CCL2 | CCR2 | MacCx3cr1 | MacCx3cr1 | 1.161 | 0 |
| CCL2 | CCR2 | MacCx3cr1 | MacCxcl9 | 1.161 | 0 |
| CCL2 | CCR2 | DCmoDCcDC2 | MacTrem2 | 1.158 | 0 |
| CD28 | CD86 | CD4 | MacFn1 | 1.157 | 0 |
| CD28 | CD86 | CD4 | MacLyve1 | 1.157 | 0 |
| CD48 | CD244 | CD8 | NK2 | 1.155 | 0 |
| CCL3 | CCR5 | MacCxcl9 | MacTrem2 | 1.153 | 0 |
| CD28 | CD86 | CD4 | MacCxcl9 | 1.152 | 0 |
| CCL2 | CCR2 | MacCx3cr1 | MacTrem2 | 1.149 | 0 |
| CCL2 | CCR2 | MacCxcl9 | MacLyve1 | 1.149 | 0 |
| CCL2 | CCR2 | pDC | MacCx3cr1 | 1.145 | 0 |
| CCL2 | CCR2 | DCmoDCcDC2 | MacLyve1 | 1.142 | 0 |
| CCL2 | CCR2 | pDC | DCmoDCcDC2 | 1.138 | 0 |
| CCL3 | CCR5 | DCmoDCcDC2 | NK1 | 1.137 | 0.037 |
| CCL2 | CCR2 | Treg | Treg | 1.134 | 0 |
| CCL2 | CCR2 | MacCx3cr1 | MacLyve1 | 1.133 | 0 |
| CCL24 | CCR2 | DCcDC1 | NK1 | 1.133 | 0 |
| CCL3 | CCR5 | DCCcr7 | MacFn1 | 1.132 | 0.039 |
| CCL24 | CCR2 | DCCcr7 | NK1 | 1.123 | 0 |
| CCL3 | CCR5 | MacCx3cr1 | MacFn1 | 1.121 | 0.002 |
| CCL3 | CCR5 | MacCx3cr1 | NK1 | 1.12 | 0.012 |
| CCL2 | CCR2 | CD8 | Treg | 1.118 | 0 |
| CCL3 | CCR5 | MacCx3cr1 | MacTrem2 | 1.111 | 0.009 |
| CCL24 | CCR2 | DCmoDCcDC2 | NK1 | 1.107 | 0 |
| CCL2 | CCR2 | MacLyve1 | MacTrem2 | 1.1 | 0 |
| CCL24 | CCR2 | MacCxcl9 | NK1 | 1.1 | 0 |
| CCL24 | CCR2 | MacCx3cr1 | NK1 | 1.091 | 0 |
| CCL24 | CCR2 | DCcDC1 | NK2 | 1.068 | 0 |
| CCL24 | CCR2 | DCCcr7 | NK2 | 1.059 | 0 |
| CD48 | CD244 | CD4 | NK2 | 1.053 | 0 |
| CCL24 | CCR2 | DCmoDCcDC2 | NK2 | 1.043 | 0 |
| CCL24 | CCR2 | MacCxcl9 | NK2 | 1.035 | 0 |
| CCL2 | CCR2 | Neutrophils | NK1 | 1.034 | 0 |
| CD28 | CD80 | CD4 | DCcDC1 | 1.033 | 0 |
| CCL2 | CCR2 | Treg | NK1 | 1.03 | 0 |
| CCL24 | CCR2 | MacCx3cr1 | NK2 | 1.027 | 0 |
| CD28 | CD80 | CD4 | MacCxcl9 | 1.024 | 0 |
| CD28 | CD80 | CD4 | MacTrem2 | 1.015 | 0 |
| CCL2 | CCR2 | CD8 | NK1 | 1.014 | 0 |
| CCL2 | CCR2 | CD4 | NK1 | 1.006 | 0 |
| CD28 | CD80 | CD4 | MacFn1 | 1.006 | 0 |
| CD48 | CD244 | CD8 | NK1 | 1.003 | 0 |
| CD28 | CD80 | CD4 | DCCcr7 | 1 | 0 |
| CCL2 | CCR2 | DCCcr7 | pDC | 0.992 | 0.003 |
| CCL2 | CCR2 | NK2 | NK1 | 0.987 | 0 |
| CCL24 | CCR2 | DCcDC1 | DCCcr7 | 0.987 | 0 |
| CCL2 | CCR2 | NK1 | NK1 | 0.982 | 0 |
| CD48 | CD244 | NK1 | NK2 | 0.979 | 0 |
| CCL24 | CCR2 | DCCcr7 | DCCcr7 | 0.977 | 0 |
| CCL24 | CCR2 | MacFn1 | DCCcr7 | 0.974 | 0 |
| CD28 | CD80 | CD4 | MacLyve1 | 0.974 | 0 |
| CCL2 | CCR2 | DCcDC1 | pDC | 0.971 | 0.003 |
| CCL2 | CCR2 | Neutrophils | NK2 | 0.97 | 0 |
| CCL2 | CCR2 | Treg | NK2 | 0.966 | 0 |
| CCL24 | CCR2 | DCmoDCcDC2 | DCCcr7 | 0.962 | 0 |
| CCL24 | CCR2 | MacCxcl9 | DCCcr7 | 0.954 | 0 |
| CCL2 | CCR2 | CD8 | NK2 | 0.95 | 0 |
| CCL24 | CCR2 | DCcDC1 | MacFn1 | 0.947 | 0 |
| CCL2 | CCR2 | MacTrem2 | pDC | 0.946 | 0.006 |
| CCL24 | CCR2 | MacCx3cr1 | DCCcr7 | 0.946 | 0 |
| CCL24 | CCR2 | pDC | DCCcr7 | 0.944 | 0 |
| CCL2 | CCR2 | CD4 | NK2 | 0.942 | 0 |
| CCL2 | CCR2 | MacCxcl9 | pDC | 0.942 | 0.007 |
| CD28 | CD80 | CD8 | DCCcr7 | 0.939 | 0 |
| CCL24 | CCR2 | DCCcr7 | MacFn1 | 0.937 | 0 |
| CTLA4 | CD80 | CD4 | Neutrophils | 0.936 | 0 |
| CCL2 | CCR2 | DCmoDCcDC2 | pDC | 0.935 | 0.007 |
| CD48 | CD244 | CD8 | Neutrophils | 0.931 | 0 |
| CCL2 | CCR2 | DCCcr7 | Mast | 0.93 | 0 |
| CCL24 | CCR2 | DCcDC1 | MacCx3cr1 | 0.928 | 0 |
| CCL24 | CCR2 | DCcDC1 | MacCxcl9 | 0.928 | 0 |
| CCL2 | CCR2 | MacCx3cr1 | pDC | 0.926 | 0.008 |
| CCL2 | CCR2 | NK2 | NK2 | 0.923 | 0.003 |
| CCL24 | CCR2 | DCmoDCcDC2 | MacFn1 | 0.921 | 0 |
| CCL2 | CCR2 | NK1 | NK2 | 0.918 | 0 |
| CCL24 | CCR2 | DCCcr7 | MacCx3cr1 | 0.918 | 0 |
| CCL24 | CCR2 | DCCcr7 | MacCxcl9 | 0.918 | 0 |
| CCL24 | CCR2 | DCcDC1 | MacTrem2 | 0.915 | 0 |
| CCL24 | CCR2 | MacFn1 | MacCx3cr1 | 0.915 | 0 |
| CCL24 | CCR2 | MacFn1 | MacCxcl9 | 0.915 | 0 |
| CCL24 | CCR2 | MacCxcl9 | MacFn1 | 0.914 | 0 |
| CD48 | CD244 | MacLyve1 | NK2 | 0.912 | 0 |
| CCL2 | CCR2 | DCcDC1 | Mast | 0.908 | 0 |
| CD48 | CD244 | DCcDC1 | NK2 | 0.907 | 0 |
| CCL24 | CCR2 | DCCcr7 | MacTrem2 | 0.906 | 0 |
| CCL24 | CCR2 | MacCx3cr1 | MacFn1 | 0.905 | 0 |
| CCL24 | CCR2 | DCmoDCcDC2 | MacCxcl9 | 0.903 | 0 |
| CCL24 | CCR2 | DCmoDCcDC2 | MacCx3cr1 | 0.902 | 0 |
| CD48 | CD244 | CD4 | NK1 | 0.901 | 0 |
| CCL24 | CCR2 | DCcDC1 | MacLyve1 | 0.899 | 0 |
| CD48 | CD244 | DCmoDCcDC2 | NK2 | 0.899 | 0 |
| CD48 | CD244 | MacFn1 | NK2 | 0.899 | 0 |
| C3 | C3AR1 | DCmoDCcDC2 | MacTrem2 | 0.897 | 0 |
| CCL24 | CCR2 | MacCxcl9 | MacCx3cr1 | 0.895 | 0 |
| CCL24 | CCR2 | MacCxcl9 | MacCxcl9 | 0.895 | 0 |
| C3 | C3AR1 | MacCxcl9 | MacTrem2 | 0.894 | 0 |
| CD48 | CD244 | MacCxcl9 | NK2 | 0.893 | 0 |
| CCL24 | CCR2 | DCCcr7 | MacLyve1 | 0.89 | 0 |
| CCL24 | CCR2 | DCmoDCcDC2 | MacTrem2 | 0.89 | 0 |
| C3 | C3AR1 | DCCcr7 | MacTrem2 | 0.889 | 0 |
| C3 | C3AR1 | DCmoDCcDC2 | MacLyve1 | 0.835 | 0 |
| CTLA4 | CD86 | CD4 | DCcDC1 | 0.835 | 0 |
| C3 | C3AR1 | MacCxcl9 | MacLyve1 | 0.832 | 0 |
| CD48 | CD244 | CD4 | Neutrophils | 0.829 | 0 |
| CCL2 | CCR2 | CD8 | MacFn1 | 0.828 | 0 |
| C3 | C3AR1 | DCCcr7 | MacLyve1 | 0.827 | 0 |
| CD48 | CD244 | NK1 | NK1 | 0.827 | 0 |
| CCL2 | CCR2 | Treg | MacCx3cr1 | 0.825 | 0 |
| CD28 | CD80 | CD4 | Mast | 0.825 | 0 |
| C3 | C3AR1 | MacCx3cr1 | MacLyve1 | 0.824 | 0 |
| C3 | C3AR1 | DCcDC1 | MacFn1 | 0.822 | 0 |
| CCL2 | CCR2 | CD4 | MacFn1 | 0.82 | 0 |
| CCL2 | CCR2 | pDC | CD8 | 0.819 | 0.033 |
| CCL2 | CCR2 | Treg | DCmoDCcDC2 | 0.818 | 0 |
| CCL2 | CCR2 | Neutrophils | MacTrem2 | 0.817 | 0 |
| NAMPT | P2RY6 | DCCcr7 | MacFn1 | 0.816 | 0 |
| CCL2 | CCR2 | MacLyve1 | Mast | 0.814 | 0 |
| CCL2 | CCR2 | Treg | MacTrem2 | 0.813 | 0 |
| CCL2 | CCR2 | CD8 | MacCx3cr1 | 0.809 | 0 |
| CCL2 | CCR2 | CD8 | MacCxcl9 | 0.809 | 0 |
| CD28 | CD80 | CD4 | CD8 | 0.807 | 0 |
| COPA | P2RY6 | pDC | MacCx3cr1 | 0.807 | 0 |
| SPN | ICAM1 | Neutrophils | CD8 | 0.804 | 0 |
| COPA | P2RY6 | pDC | MacTrem2 | 0.802 | 0 |
| CCL2 | CCR2 | Neutrophils | MacLyve1 | 0.801 | 0 |
| CCL2 | CCR2 | NK2 | MacFn1 | 0.801 | 0.001 |
| SPN | ICAM1 | pDC | CD8 | 0.8 | 0 |
| CTLA4 | CD86 | CD4 | MacTrem2 | 0.799 | 0 |
| NAMPT | P2RY6 | DCCcr7 | MacTrem2 | 0.798 | 0 |
| CCL2 | CCR2 | CD8 | MacTrem2 | 0.797 | 0 |
| CCL2 | CCR2 | Treg | MacLyve1 | 0.797 | 0 |
| CCL2 | CCR2 | NK1 | MacFn1 | 0.796 | 0 |
| CD72 | SEMA4D | Bcells | CD8 | 0.79 | 0 |
| PDGFB | LRP1 | NK1 | MacTrem2 | 0.79 | 0 |
| C3 | C3AR1 | DCcDC1 | MacLyve1 | 0.789 | 0 |
| CCL2 | CCR2 | CD4 | MacTrem2 | 0.789 | 0 |
| NAMPT | P2RY6 | MacCx3cr1 | MacFn1 | 0.789 | 0 |
| COPA | P2RY6 | pDC | pDC | 0.788 | 0 |
| PDGFB | LRP1 | DCcDC1 | MacLyve1 | 0.788 | 0 |
| CTLA4 | CD86 | CD4 | MacLyve1 | 0.787 | 0 |
| NAMPT | P2RY6 | DCmoDCcDC2 | MacFn1 | 0.787 | 0 |
| CTLA4 | CD86 | CD4 | MacFn1 | 0.786 | 0 |
| SPN | ICAM1 | MacLyve1 | CD8 | 0.785 | 0 |
| CTLA4 | CD86 | CD4 | MacCxcl9 | 0.782 | 0 |
| CCL2 | CCR2 | CD8 | MacLyve1 | 0.781 | 0 |
| CCL2 | CCR2 | CD4 | MacLyve1 | 0.773 | 0 |
| NAMPT | P2RY6 | DCcDC1 | MacFn1 | 0.773 | 0 |
| NAMPT | P2RY6 | MacCxcl9 | MacFn1 | 0.773 | 0 |
| PDGFB | LRP1 | MacCxcl9 | MacLyve1 | 0.773 | 0 |
| PDGFB | LRP1 | DCCcr7 | MacLyve1 | 0.772 | 0 |
| NAMPT | P2RY6 | MacCx3cr1 | MacTrem2 | 0.771 | 0 |
| CCL2 | CCR2 | NK2 | MacTrem2 | 0.77 | 0.003 |
| NAMPT | P2RY6 | DCmoDCcDC2 | MacTrem2 | 0.769 | 0 |
| PDGFB | LRP1 | MacCx3cr1 | MacLyve1 | 0.766 | 0 |
| CCL2 | CCR2 | NK1 | MacTrem2 | 0.765 | 0 |
| CD48 | CD244 | CD8 | Mast | 0.765 | 0 |
| CD48 | CD244 | MacLyve1 | NK1 | 0.76 | 0 |
| NAMPT | P2RY6 | DCCcr7 | MacLyve1 | 0.758 | 0.002 |
| CD48 | CD244 | DCcDC1 | NK1 | 0.755 | 0 |
| CD48 | CD244 | NK1 | Neutrophils | 0.755 | 0 |
| NAMPT | P2RY6 | DCcDC1 | MacTrem2 | 0.755 | 0 |
| NAMPT | P2RY6 | MacCxcl9 | MacTrem2 | 0.755 | 0 |
| PDGFB | LRP1 | DCcDC1 | MacTrem2 | 0.755 | 0 |
| CCL2 | CCR2 | NK2 | MacLyve1 | 0.754 | 0.015 |
| CD48 | CD244 | CD4 | MacCxcl9 | 0.754 | 0 |
| PDGFB | LRP1 | CD8 | MacLyve1 | 0.752 | 0 |
| C3 | C3AR1 | MacCx3cr1 | MacTrem2 | 0.886 | 0 |
| CCL24 | CCR2 | MacCx3cr1 | MacCx3cr1 | 0.886 | 0 |
| CCL24 | CCR2 | MacCx3cr1 | MacCxcl9 | 0.886 | 0 |
| CCL24 | CCR2 | pDC | MacCx3cr1 | 0.885 | 0 |
| CCL2 | CCR2 | MacTrem2 | Mast | 0.883 | 0 |
| CCL24 | CCR2 | MacCxcl9 | MacTrem2 | 0.882 | 0 |
| CD48 | CD244 | MacCx3cr1 | NK2 | 0.881 | 0 |
| CCL2 | CCR2 | MacCxcl9 | Mast | 0.88 | 0 |
| CCL24 | CCR2 | pDC | DCmoDCcDC2 | 0.878 | 0 |
| CCL2 | CCR2 | MacLyve1 | pDC | 0.877 | 0.014 |
| SPN | ICAM1 | Treg | CD8 | 0.875 | 0 |
| CCL24 | CCR2 | DCmoDCcDC2 | MacLyve1 | 0.874 | 0 |
| CCL24 | CCR2 | MacCx3cr1 | MacTrem2 | 0.874 | 0 |
| CCL2 | CCR2 | DCmoDCcDC2 | Mast | 0.873 | 0 |
| C3 | C3AR1 | DCmoDCcDC2 | MacFn1 | 0.869 | 0 |
| CD48 | CD244 | Bcells | NK2 | 0.869 | 0 |
| C3 | C3AR1 | MacCxcl9 | MacFn1 | 0.866 | 0 |
| CCL24 | CCR2 | MacCxcl9 | MacLyve1 | 0.866 | 0 |
| CCL2 | CCR2 | MacCx3cr1 | Mast | 0.863 | 0 |
| C3 | C3AR1 | DCCcr7 | MacFn1 | 0.861 | 0 |
| CD48 | CD244 | pDC | NK2 | 0.86 | 0 |
| CCL24 | CCR2 | MacCx3cr1 | MacLyve1 | 0.858 | 0 |
| C3 | C3AR1 | MacCx3cr1 | MacFn1 | 0.857 | 0 |
| CD48 | CD244 | CD8 | MacCxcl9 | 0.856 | 0 |
| CD48 | CD244 | DCCcr7 | NK2 | 0.856 | 0 |
| CCL2 | CCR2 | MacTrem2 | CD8 | 0.854 | 0 |
| CD48 | CD244 | CD8 | MacLyve1 | 0.852 | 0 |
| C3 | C3AR1 | DCcDC1 | MacTrem2 | 0.851 | 0 |
| CCL2 | CCR2 | Neutrophils | MacFn1 | 0.848 | 0 |
| CD48 | CD244 | CD8 | MacTrem2 | 0.847 | 0 |
| CD28 | CD80 | CD4 | Treg | 0.846 | 0 |
| CCL2 | CCR2 | Treg | MacFn1 | 0.844 | 0 |
| CTLA4 | CD80 | CD8 | DCCcr7 | 0.842 | 0 |
| CD48 | CD244 | CD8 | MacFn1 | 0.837 | 0 |

### REFERENCES

1. R. D. Paladini, Takahashi K., Bravo N. S., Coulombe P. A., Onset of re-epithelialization after skin injury correlates with a reorganization of keratin filaments in wound edge keratinocytes: defining a potential role for keratin 16. Journal of Cell Biology 132, 381 (1996).
2. D. D. J. Hobbs R. P., Jacob J. T., Han M. C., Chung B. M., Batazzi A. S., Poll B. J., Guo Y., Han J., Ong S., Zheng W., Taube J. M., Čiháková D., Wan F., Coulombe P. A., Keratin-dependent regulation of Aire and gene expression in skin tumor keratinocytes. Nature Genetics 47, 933-938 (2015).
3. M. Komine, I. M. Freedberg, M. Blumenberg, Regulation of epidermal expression of keratin K17 in inflammatory skin diseases. J Invest Dermatol 107, 569-575 (1996).
4. K. M. McGowan, P. A. Coulombe, Keratin 17 expression in the hard epithelial context of the hair and nail, and its relevance for the pachyonychia congenita phenotype. J Invest Dermatol 114, 1101-1107 (2000).
5. C. P. A. McGowan K. M., Onset of keratin 17 expression coincides with the definition of major epithelial lineages during skin development. J Cell Biol 143, 469-486 (1998).
6. D. DePianto, Kerns M., Dlugosz A. A., Coulombe P. A., Keratin 17 promotes epithelial proliferation and tumor growth by polarizing the immune response in skin. Nature Genetics 42, 910-914 (2010).
7. L. F. Escobar-Hoyos, Yang J., Zhu J., Cavallo J. A., Zhai H., Burke S., Koller A., Chen E. I., Shroyer K. R., Keratin 17 in premalignant and malignant squamous lesions of the cervix: proteomic discovery and immunohistochemical validation as a diagnostic and prognostic biomarker. Mod Pathol 27, 621-630 (2014).
8. R. Khanom, Nguyen C. T. K., Kayamori K., Zhao X., Morita K., Miki Y., Katsube K., Yamaguchi A., Sakamoto K., Keratin 17 Is Induced in Oral Cancer and Facilitates Tumor Growth. PLOS One 11, (2016).
9. R. D. Merkin, Vanner E. A., Romeiser J. L., Shroyer A. L. W., Escobar-Hoyos L. F., Li J., Powers R. S., Burke S., Shroyer K. R., Keratin 17 is overexpressed and predicts poor survival in estrogen receptor-negative/human epidermal growth factor receptor-2-negative breast cancer. Human Pathology 62, 23-32 (2016).
10. E. Regenbogen, Mo M., Romeiser J, Shroyer A. L. W., Escobar-Hoyos L. F., Burke S., Shroyer K. R., Elevated expression of keratin 17 in oropharyngeal squamous cell carcinoma is associated with decreased survival. Head and Neck 40, 1788-1798 (2018).
11. Y. F. Wang et al., Overexpression of keratin 17 is associated with poor prognosis in epithelial ovarian cancer. Tumour Biol 34, 1685-1689 (2013).
12. R. P. Hobbs, Batazzi A. S., Han M. C., Coulombe P. A., Loss of Keratin 17 induces tissue-specific cytokine polarization and cellular differentiation in HPV16-driven cervical tumorigenesis in vivo. Oncogene 35, 5653-5662 (2016).
13. W. Wang, Uberoi A., Spurgeon M., Gronski E., Majerciak V., Lobanov A., Hayes M., Loke A., Zheng Z. M., Lambert P. F., Stress keratin 17 enhances papillomavirus infection-induced disease by downregulating T cell recruitment. PLoS Pathog 16, (2020).
14. H. Kim et al., Clinical outcomes of immune checkpoint inhibitors for patients with recurrent or metastatic head and neck cancer: real-world data in Korea. BMC Cancer 20, 727 (2020).
15. P. C. Tumeh, Harview C. L., Yearley J. H., Shintaku I. P., Taylor E. J. M., Robert L., Chmielowski B., Spasic M., Henry G., Ciobanu V., West, A. N. Carmona M., Kivork C., Seja E., Cherry G., Gutierrez A., Grogan T. R., Mateus C., Tomasic G., Glaspy J. A., Emerson R. O., Robins H., Pierce R. H., Elashoff D. A., Robert C., Ribas A., PD-1 blockade induces responses by inhibiting adaptive immune resistance. Nature, (2014).
16. N. P. Judd et al., ERK1/2 regulation of CD44 modulates oral cancer aggressiveness. Cancer Res 72, 365-374 (2012).
17. N. P. Judd, C. T. Allen, A. E. Winkler, R. Uppaluri, Comparative analysis of tumor-infiltrating lymphocytes in a syngeneic mouse model of oral cancer. Otolaryngol Head Neck Surg 147, 493-500 (2012).
18. P. Zolkind et al., Cancer immunogenomic approach to neoantigen discovery in a checkpoint blockade responsive murine model of oral cavity squamous cell carcinoma. Oncotarget 9, 4109-4119 (2018).
19. S. W. Wingett, S. Andrews, FastQ Screen: A tool for multi-genome mapping and quality control. F1000Res 7, 1338 (2018).
20. Y. Liao, G. K. Smyth, W. Shi, The Subread aligner: fast, accurate and scalable read mapping by seed-and-vote. Nucleic Acids Res 41, e108 (2013).
21. Y. Liao, G. K. Smyth, W. Shi, featureCounts: an efficient general purpose program for assigning sequence reads to genomic features. Bioinformatics 30, 923-930 (2014).
22. C. W. Law, Y. Chen, W. Shi, G. K. Smyth, voom: Precision weights unlock linear model analysis tools for RNA-seq read counts. Genome Biol 15, R29 (2014).
23. M. D. Robinson, D. J. McCarthy, G. K. Smyth, edgeR: a Bioconductor package for differential expression analysis of digital gene expression data. Bioinformatics 26, 139-140 (2010).
24. Z. Gu, R. Eils, M. Schlesner, Complex heatmaps reveal patterns and correlations in multidimensional genomic data. Bioinformatics 32, 2847-2849 (2016).
25. C. S. McGinnis, L. M. Murrow, Z. J. Gartner, DoubletFinder: Doublet Detection in Single-Cell RNA Sequencing Data Using Artificial Nearest Neighbors. Cell Syst 8, 329-337 e324 (2019).
26. I. Korsunsky et al., Fast, sensitive and accurate integration of single-cell data with Harmony. Nat Methods 16, 1289-1296 (2019).
27. T. Stuart et al., Comprehensive Integration of Single-Cell Data. Cell 177, 1888-1902 e1821 (2019).
28. G. Finak et al., MAST: a flexible statistical framework for assessing transcriptional changes and characterizing heterogeneity in single-cell RNA sequencing data. Genome Biol 16, 278 (2015).
29. M. Efremova, M. Vento-Tormo, S. A. Teichmann, R. Vento-Tormo, CellPhoneDB: inferring cell-cell communication from combined expression of multi-subunit ligand-receptor complexes. Nat Protoc 15, 1484-1506 (2020).
30. S. Aibar et al., SCENIC: single-cell regulatory network inference and clustering. Nat Methods 14, 1083-1086 (2017).
31. A. Subramanian et al., Gene set enrichment analysis: a knowledge-based approach for interpreting genome-wide expression profiles. Proc Natl Acad Sci USA 102, 15545-15550 (2005).
32. S. M. Gordon et al., The transcription factors T-bet and Eomes control key checkpoints of natural killer cell maturation. Immunity 36, 55-67 (2012).
33. J. D. K. Bai et al., Keratin 17 is a negative prognostic biomarker in high-grade endometrial carcinomas. Hum Pathol 94, 40-50 (2019).
34. L. Roa-Pena et al., Keratin 17 identifies the most lethal molecular subtype of pancreatic cancer. Sci Rep 9, 11239 (2019).
35. Y. Zeng et al., Keratin 17 Suppresses Cell Proliferation and Epithelial-Mesenchymal Transition in Pancreatic Cancer. Front Med (Lausanne) 7, 572494 (2020).
36. M. T. G. Ryan R. Kawalerski, Lucia Roa-Peña, Cindy V. Leiton, Chun-Hao Pan, Luke A. Torre-Healy, Taryn Boyle, Natasha T. Snider, Kenneth R. Shroyer and Luisa F. Escobar-Hoyos, Abstract PO-050: Solubilization of keratin 17 promotes the metastatic dissemination of the most lethal form of pancreatic cancer. Cancer Research, (2020).
37. J. E. Grossman, D. Vasudevan, C. E. Joyce, M. Hildago, Is PD-L1 a consistent biomarker for anti-PD-1 therapy? The model of balstilimab in a virally-driven tumor. Oncogene 40, 1393-1395 (2021).
38. R. Mehra et al., Efficacy and safety of pembrolizumab in recurrent/metastatic head and neck squamous cell carcinoma: pooled analyses after long-term follow-up in KEYNOTE-012. Br J Cancer 119, 153-159 (2018).
39. R. W. Naumann et al., Safety and Efficacy of Nivolumab Monotherapy in Recurrent or Metastatic Cervical, Vaginal, or Vulvar Carcinoma: Results From the Phase I/II CheckMate 358 Trial. J Clin Oncol 37, 2825-2834 (2019).
40. S. Crosta et al., PD-L1 Testing and Squamous Cell Carcinoma of the Head and Neck: A Multicenter Study on the Diagnostic Reproducibility of Different Protocols. Cancers (Basel) 13, (2021).
41. A. Bassez et al., A single-cell map of intratumoral changes during anti-PD1 treatment of patients with breast cancer. Nat Med 27, 820-832 (2021).
42. E. Moore et al., Established T Cell-Inflamed Tumors Rejected after Adaptive Resistance Was Reversed by Combination STING Activation and PD-1 Pathway Blockade. Cancer Immunol Res 4, 1061-1071 (2016).
43. M. D. Onken et al., A surprising cross-species conservation in the genomic landscape of mouse and human oral cancer identifies a transcriptional signature predicting metastatic disease. Clin Cancer Res 20, 2873-2884 (2014).
44. B. M. Chung et al., Regulation of C-X-C chemokine gene expression by keratin 17 and hnRNP K in skin tumor keratinocytes. J Cell Biol 208, 613-627 (2015).
45. I. G. House et al., Macrophage-Derived CXCL9 and CXCL10 Are Required for Antitumor Immune Responses Following Immune Checkpoint Blockade. Clin Cancer Res 26, 487-504 (2020).
46. D. Dangaj et al., Cooperation between Constitutive and Inducible Chemokines Enables T Cell Engraftment and Immune Attack in Solid Tumors. Cancer Cell 35, 885-900 e810 (2019).
47. K. Litchfield et al., Meta-analysis of tumor-and T cell-intrinsic mechanisms of sensitization to checkpoint inhibition. Cell 184, 596-614 e514 (2021).
48. R. Cristescu et al., Pan-tumor genomic biomarkers for PD-1 checkpoint blockade-based immunotherapy. Science 362, (2018).
49. M. Zhang et al., CCL7 recruits cDC1 to promote antitumor immunity and facilitate checkpoint immunotherapy to non-small cell lung cancer. Nat Commun 11, 6119 (2020).
50. B. Huang et al., CCL2/CCR2 pathway mediates recruitment of myeloid suppressor cells to cancers. Cancer Lett 252, 86-92 (2007).
51. B. Z. Qian et al., CCL2 recruits inflammatory monocytes to facilitate breast-tumour metastasis. Nature 475, 222-225 (2011).
52. H. Seo et al., BATF and IRF4 cooperate to counter exhaustion in tumor-infiltrating CAR T cells. Nat Immunol 22, 983-995 (2021).

We claim:

1. A method of determining responsiveness of a cancer to immunotherapy in a subject, the method comprising:
(a) obtaining a sample from the subject; and
(b) detecting the expression level of keratin 17 (K17) in the sample; wherein a low level of K17 expression indicates that the cancer is responsive to the immunotherapy.

2. The method of claim 1, further comprising: (c) treating the subject with cancer with an immunotherapy when low levels of K17 are detected in step (b).

3. The method of claim 1, further comprising: (c) detecting the expression level of at least one additional marker in the sample; wherein a low level of K17 expression and detection of the at least one additional marker indicates that the cancer is responsive to the immunotherapy.

4. The method of claim 3, wherein the at least one additional marker is selected from the group consisting of IFNγ, CXCL10, CXCL11, PD-L1, CCL15, CCL23, CCL7, CXCL9, ENTPD1, SIRPG, ADORA2A, CCR1, CD47, CXCR3, DPP4, and FCGR2A.

5. The method of claim 4, wherein the at least one additional marker is PD-L1.

6. The method of any one of claims 3-5, further comprising: (d) treating the subject with cancer with an immunotherapy when low levels of K17 and increased levels of the at least one additional marker expression are detected in step (c).

7. The method of claim 1, further comprising: (c) detecting the expression level of at least one additional marker in the sample; wherein a low level of K17 expression and a low level of the at least one additional marker expression indicates that the cancer is responsive to the immunotherapy.

8. The method of claim 7, wherein the at least one additional marker is selected from the group consisting of CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, and SPN.

9. The method of any one of claims 7-8, further comprising: (d) treating the subject with cancer with an immunotherapy when low levels of K17 and of the at least one additional marker expression are detected in step (c).

10. The method of any one of claim 2, 6, or 9, wherein the immunotherapy is an immune checkpoint inhibitor.

11. The method of claim 10, wherein the immune checkpoint inhibitor is a PD-1 inhibitor, a PD-L1 inhibitor, or a CTLA4 inhibitor.

12. A method of predicting if a cancer is non-responsive to an immunotherapy, the method comprising:
(a) obtaining a sample from a subject; and
(b) detecting the expression level in the sample of keratin 17 (K17); wherein detection of a high level of expression of K17 indicates that the cancer is non-responsive to immunotherapy.

13. The method of claim 12, further comprising: (c) treating the subject with cancer with a cancer therapy that is not an immune checkpoint inhibitor when high levels of K17 are detected.

14. The method of claim 12, further comprising: (c) detecting the expression level of at least one additional marker in the sample; wherein detection of a high level of expression of K17 and increased levels of the at least one additional marker indicates that the cancer is non-responsive to immunotherapy.

15. The method of claim 14, wherein the at least one additional marker is selected from the group comprising CCL2, CCL24, CCL3, CCL4, CCR2, CCR5, CD244, CD28, CD48, CD72, CD80, CD86, COPA, CTLA4, ICAM1, LRP1, MRC1, NAMPT, P2RY6, PDGFB, PTPRC, SELL, SELPLG, SEMA4D, C3, C3AR1, and SPN.

16. The method of any one of claims 14-15, further comprising: (d) treating the subject with cancer with a cancer therapy that is not an immune checkpoint inhibitor when high levels of K17 and increased levels of the at least one additional marker are detected.

17. The method of claim 12, further comprising: (c) detecting the expression level of at least one additional marker in the sample; wherein detection of a high level of expression of K17 and decreased levels of the at least one additional marker indicates that the cancer is non-responsive to immunotherapy.

18. The method of claim 17, wherein the at least one additional marker is selected from the group comprising IFNγ, CXCL10, CXCL11, PD-L1, CCL15, CCL23, CCL7, CXCL9, ENTPD1, SIRPG, ADORA2A, CCR1, CD47, CXCR3, DPP4, and FCGR2A.

19. The method of claim 18, wherein the at least one additional marker is PD-L1.

20. The method of any one of claims 17-19, further comprising: (d) treating the subject with cancer with a cancer therapy that is not an immune checkpoint inhibitor when high levels of K17 and low levels of the at least one additional marker are detected.

21. The method of any one of claim 13, 16, or 20, wherein the cancer therapy is chemotherapy or radiation.

22. The method of any one of the preceding claims, wherein the expression level of K17 in the subject is compared to a responsive or non-responsive control sample.

23. The method of any one of claim 3-11 or 14-22, wherein the expression level of the at least one additional marker is compared to a responsive or non-responsive control sample.

24. The method of any one of claim 22 or 23, wherein the control is from a healthy subject, a non-cancerous tissue from the subject with cancer, or an established expression level.

25. The method of any one of the preceding claims, wherein the expression level is detected using RNA expression levels.

26. The method of claim 25, wherein the low level expression of K17 corresponds to at least a 300-fold decrease in K17 RNA expression levels compared to the non-responsive control.

27. The method of claim 25, wherein the low level expression of K17 corresponds to at least a 10,000-fold decrease in K17 RNA expression levels compared to the non-responsive control.

28. The method of any one of claims 1-24, wherein the expression levels are protein expression levels.

29. The method of any one of claim 1-11 or 22-28, wherein the low level expression of K17 corresponds to equal to or less than 5% of cells expressing K17.

30. The method of claim 12-21 or 23-28, wherein the high level expression of K17 corresponds to greater than 5% of cells expressing K17.

31. The method of any one of the preceding claims, wherein the cancer is head and neck cancer, skin cancer, small cell lung cancer, cervical cancer, lung squamous cell carcinoma, breast cancer, pancreatic cancer, or other epithelial originated cancer.

32. The method of any one of the preceding claims, wherein the sample is a tumor sample or biopsy sample.
